(19) United States
(12) Patent Application Publication
(10) Pub. No.: US 2025/0129394 A1
(43) Pub. Date: Apr. 24, 2025

(54) CELLULAR ENGINEERING TO IMPROVE CANNABINOID PRODUCTION IN MICROBIAL CELLS

(71) Applicant: Cellibre, Inc., San Diego, CA (US)

(72) Inventors: Carolyn J. Collins, San Diego, CA (US); Diep Minh Ngoc Nguyen, San Diego, CA (US); Jun Urano, Irvine, CA (US); Nicky Christopher Caiazza, Encinitas, CA (US); Spiros Kambourakis, San Diego, CA (US)

(21) Appl. No.: 18/701,608

(22) PCT Filed: Oct. 17, 2022
(86) PCT No.: PCT/US2022/046926

§ 371 Date: Apr. 15, 2024

Related U.S. Application Data
(60) Provisional application No. 63/256,398, filed on Oct. 15, 2021.

(51) Int. Cl. C12P 7/42; C12N 9/00; C12N 9/02; C12N 9/10; C12N 9/88; C12N 15/52

(52) U.S. Cl. CPC C12P 7/42; C12N 9/0008; C12N 9/1085; C12N 9/88; C12N 9/93; C12N 15/52; C12Y 102/01004; C12Y 205/0101; C12Y 401/01001; C12Y 602/01001; C12Y 604/01002

(57) ABSTRACT

Provided herein are enzymes, cells, and methods to optimize the production of cannabinoids in micro-organisms.

### RELATED APPLICATIONS

This application claims the benefit of U.S. Provisional Application No. 63/256,398, filed Oct. 15, 2021, the entire teachings of which are incorporated herein by reference.

### BACKGROUND OF THE INVENTION

The Cannabaceae family of plants produces numerous different cannabinoids (>=120) in variable, relative quantities over a 7-10 week flowering period. Many of these cannabinoids have and are currently being explored as therapeutics in chordates (e.g., mammals), and as a result, they are largely approved (>=35 states) for either medical and/or recreational use in the United States. There remains a need for the production of cannabinoids in micro-organisms in order to increase yield and purity of the desired cannabinoids.

### SUMMARY OF THE INVENTION

One aspect of the invention makes chemicals or other biomolecules that are produced using enzymes that utilize GPP as a substrate. Such molecules include monoterpenes (e.g. geraniol, pinene, limonene, etc.) that are made through the action of different synthases on GPP, or other molecules that at some point during biosynthesis undergo prenylation with GPP (e.g. cannabinoids [CBGA, CBGVA, THCA, etc.], or other chemicals and bioactive molecules [de Bruijn et al Trends Biotechnol 2020, 38 (8), 917-934; Chen, X, et al, Pharm Biol. 2014, 52 (5), 655-660]). As one aspect of this technology results in a strain with improved flux to GPP, it follows that there is an increase in production of the GPP precursor, DMAPP. Thus, molecules derived from prenylation by DMAPP can also be made using this technology. Furthermore, this technology can be used to prenylate proteins. The other aspect of the invention is that it can be used to enhance production of Acyl-CoA's, such as Acetyl-CoA, Malonyl-CoA, Butyrl-CoA, and Hexanoyl-CoA. Both GPP and acyl-CoA's are key precursor molecules for cannabinoid biosynthesis.

Some aspects of the present disclosure are related to a cell producing an increased ratio of geranyl diphosphate (GPP) to farnesyl diphosphate (FPP) as compared to a control cell, wherein the cell expresses a mutant farnesyl pyrophosphate synthase protein (FPPS). In some embodiments, the cell produces an increased level of GPP as compared to a control cell.

In some embodiments, the mutant FPPS is a mutant ERG20 with at least one insertion, deletion, or substitution at a position selected from positions 88-90 of wild-type ERG20 (SEQ ID NO: 1). In some embodiments, the mutant ERG20 is ERG20.A28 (e.g., SEQ ID NO: 22) or a mutant ERG20 with at least about 90% homology to wild-type ERG20 (SEQ ID NO: 1) and comprising at least one insertion, deletion, or substitution at a position selected from positions 88-90 of wild-type ERG20 (SEQ ID NO: 1). In some embodiments, the mutant FPPS is a mutant ERG20 homolog or ortholog with an amino acid sequence at least about 90% homologous to the amino acid sequence of wild-type ERG20 (SEQ ID NO: 1) and comprises at least one insertion, deletion, or substitution at an amino acid position equivalent to one or more amino acid positions 88-90 of wild-type ERG20 (SEQ ID NO: 1).

In some embodiments, the cell has altered expression of the mutant FPPS as compared to expression of wild-type FPPS in a control cell. In some embodiments, the cell has reduced or no expression of wild-type ERG20. In some embodiments, the cell expresses ERG20.A28 (e.g., SEQ ID NO: 22) and at least one of ERG20WW (i.e., ERG20.F88W.N119W, e.g., SEQ ID NO: 32), or a ERG20WW-fused with a membrane bound or soluble prenyltransferase at its N-terminus or C-terminus, with or without a linker (e.g., MPT4.1, SEQ ID NO: 47; MPT21.9, SEQ ID NO: 48; APT73.81, SEQ ID NO:49, e.g., as described in co-owned U.S. provisional application 63/188,648, hereby incorporated by reference in its entirety). Exemplary membrane bound prenyltransferases that may be fused to an ERG20 enzyme include those proteins with prenyltransferase activity with amino acid sequences comprising at least 80%, at least 90%, at least 95%, at least 97%, or at least 99% identity with the amino acid sequences of SEQ ID NOs: 47, 48 and 53-75. Exemplary membrane aromatic soluble prenyltransferases that may be fused to an ERG20 enzyme include those proteins comprising prenyltransferase activity with amino acid sequences comprising at least 80%, at least 90%, at least 95%, at least 97%, or at least 99% identity with the amino acid sequences of SEQ ID NOs: 49 and 76-78. In some embodiments, the ERG20 enzyme may be fused to a membrane-bound or soluble prentyltransferase through a linker that comprise an amino acid sequence with at least 90% identity to the amino acid sequences of SEQ ID NOs: 79-96. In some embodiments, the ERG20 In other embodiments the cell expresses ERG20.A28 and a geranyl diphosphate synthase (GPPS, EC 2.5.1.1. i.e., AgGPPS_truncated (SEQ ID NO:36) and CgGGPPS (SEQ ID NO:37)) or a farnesyl pyrophosphate synthase protein (FPPS) having a greater preference for GPP formation over FPP formation as compared to a FPPS control.

In some embodiments, the cell has increased flux through the mevalonate (MVA) pathway as compared to a control cell. In some embodiments, the cell having increased flux through the MVA pathway over-expresses one or more native MVA pathway genes and/or expresses one or more transgenic MVA pathway genes. In some embodiments, the transgenic MVA pathway genes are selected from feedback insensitive Erg13 (HMG-COA synthase, e.g., SEQ ID NO: 3 or 4), Erg12 (mevalonate kinase, e.g., SEQ ID NO: 5 or 6), mvaE (acetyl-CoA acetyltransferase/HMG-COA reductase (NADPH), e.g., SEQ ID NO: 46) and NADH-dependent HMG-COA reductase (for example, UniProt #'s A9HWZ9 and A9BQX8). In some embodiments, the cell having increased flux through the MVA pathway, expresses ERG20.A28.

In some embodiments, the cell overexpresses mevalonate-5-phosphate decarboxylase (MPD, EC 4.1.1.99) and isopentenyl phosphokinase (IPK, EC 2.7.4.26).

In some embodiments, the cell overexpresses NADPH-dependent hydroxymethylglutaryl-CoA reductase.

In some embodiments, the cell expresses one or more transgenic genes selected from limonene monoterpene synthase (e.g., PfLS from Perilla frutescens (SEQ ID NO: 38)), myrcene monoterpene synthase (e.g., QiMyrS from Quercus ilex (SEQ ID NO: 39)) and cineole monoterpene synthase (e.g., SfCinS1 from Salvia 3ruticose (SEQ ID NO: 40)). In some embodiments, the cell has increased production of one or more monoterpenes as compared to a control cell.

In some embodiments, the cell has an elevated level of DMAPP or GPP as compared to a control cell. In some embodiments, the cell produces an elevated amount of one or more compounds prenylated with DMAPP as a donor as compared to a control cell. In some embodiments, the cell produces an elevated amount of prenylated compounds with DMAPP when expressing a DMAPP-selective prenyl transferase, as compared to the control cell.

In some embodiments, the cell overexpresses acetyl-CoA synthase (ACS, i.e., E.C. 6.2.1.1) or overexpresses both ACS and acetyl-CoA carboxylase (ACC, i.e., E.C. 6.4.1.2) as compared to a control cell. ACS and ACC may be overexpression of the native enzymes or a homolog from a heterologous system. In some embodiments, the cell produces CBGA or THCA and has increased OA production and/or CBGA production as compared to a control cell. In some embodiments, the ACS is a mutant ACS with greater specificity for converting hexanoic acid to hexanoyl-coA than the corresponding wild-type ACS. In some embodiments, the ACS is selected from the group consisting of ACS1 (SEQ ID NO: 41), ACS1.1 (SEQ ID NO: 7), and an ACS with 90% homology to ACS1.1 (SEQ ID NO: 7). In some embodiments, the ACC is a mutant ACC with increased activity compared to wild-type ACC. In some embodiments, the ACC is selected from the group consisting of ACC1 (SEQ ID NO: 44), ACC1.1 (SEQ ID NO: 45), and an ACC with 90% homology to ACC1.1 (SEQ ID NO: 45).

In some embodiments, the cell overexpresses pyruvate decarboxylase (PDC, i.e., E.C. 4.1.1.1) and/or aldehyde dehydrogenase (ALD, i.e., E.C. 1.2.1.3) as compared to a control cell.

In some embodiments, the cell overexpresses one or more non-oxidative glycolysis pathway genes, such as PTA (phosphotransacetylase, E.C. 2.3.1.8, e.g., SEQ ID NO: 42) and XPK (xylulose phosphoketolase, E.C. 4.1.2.9, e.g., SEQ ID NO: 43), and has increased cannabinoid production as compared to a control cell.

In some embodiments, the cell expresses transgenic acetylating aldehyde dehydrogenase (ADA, i.e., E.C. 1.2.1.10) and has increased cannabinoid production as compared to a control cell.

In some embodiments, the cell is a yeast cell or a bacterial cell. In some embodiments, the yeast cell is a Yarrowia strain, Saccharomyces strain or Pichia strain.

Some aspects of the present disclosure are directed to a method of producing CBGA, CBGVA, or a cannabinoid derived from CBGA or CBGVA, a monoterpene, or a monoterpenoid comprising culturing a cell as disclosed herein with a suitable carbon source under suitable conditions to produce the CBGA, monoterpene, or monoterpenoid. In some embodiments, the method further comprises isolating the CBGA, CBGVA, or a cannabinoid derived from CBGA or CBGVA, a monoterpene, or monoterpenoid from the culture.

Some aspects of the present disclosure are directed to a mutant ERG20 with at least about 90% homology to wild-type ERG20 (SEQ ID NO: 1) and comprising at least one insertion, deletion, or substitution at a position selected from positions 88-90 of wild-type ERG20 (SEQ ID NO: 1). In some embodiments, the mutant ERG20 has a polypeptide sequence selected from the group consisting of SEQ ID NOS: 8-31 or a sequence with at least 95% identity to a polypeptide sequence selected from the group consisting of SEQ ID NOS: 8-31. In some embodiments, the mutant ERG20 preferentially produces GPP over FPP.

Some aspects of the present disclosure are directed to a cell overexpressing acetyl-CoA synthase (ACS) or overexpressing both ACS and acetyl-CoA carboxylase (ACC) as compared to a control cell. In some embodiments, the cell produces CBGA, CBDA, CBCA or THCA and has increased OA production and/or CBGA, CBDA, CBCA or THCA production as compared to a control cell. In some embodiments, the ACS is a mutant ACS with greater specificity for converting hexanoic acid to hexanoyl-CoA than the corresponding wild-type ACS. In some embodiments, the ACS is selected from the group consisting of ACS1 (SEQ ID NO: 41), ACS1.1 (SEQ ID NO: 7), or an ACS with 90% homology to ACS1.1.

Some aspects of the present disclosure are directed to a cell overexpressing acetyl-CoA synthase (ACS) or overexpressing both ACS and acetyl-CoA carboxylase (ACC) as compared to a control cell. In some embodiments, the cell produces CBGVA, CBDVA, CBCVA or THCVA and has increased DVA production and/or CBGVA, CBDVA, CBCVA or THCVA production as compared to a control cell.

In some embodiments, the cell is a yeast cell or a bacterial cell. In some embodiments, the yeast cell is a Yarrowia strain, Saccharomyces strain, or Pichia strain.

Some aspects of the present disclosure are directed to a mutant acetyl-CoA synthase (ACS) selected from ACS1.1 (SEQ ID NO: 7) or an ACS with 90% homology to ACS1.1. In some embodiments, the mutant ACS has greater specificity for converting hexanoic acid to hexanoyl-coA than the corresponding wild-type ACS.

Some aspects of the present disclosure are directed to a cell (e.g., a yeast cell or a bacterial cell, a Yarrowia strain cell, Saccharomyces strain cell, or Pichia strain cell) overexpressing pyruvate decarboxylase (PDC) and/or aldehyde dehydrogenase (ALD) as compared to a control cell.

Some aspects of the present disclosure are directed to a cell (e.g., a yeast cell or a bacterial cell, a Yarrowia strain cell, Saccharomyces strain cell, or Pichia strain cell) overexpressing one or more non-oxidative glycolysis pathway genes, such as PTA (phosphotransacetylase, E.C. 2.3.1.8, e.g., SEQ ID NO: 42) or XPK (xylulose phosphoketolase, E.C. 4.1.2.9, e.g., SEQ ID NO: 43), and has increased cannabinoid production as compared to a control cell.

Some aspects of the present disclosure are directed to a cell (e.g., a yeast cell or a bacterial cell, a Yarrowia strain cell, Saccharomyces strain cell, or Pichia strain cell) overexpressing transgenic acetylating aldehyde dehydrogenase (ADA, E.C. 1.2.1.10) and has increased cannabinoid production as compared to a control cell.

All patents, patent applications, and other publications (e.g., scientific articles, books, websites, and databases) mentioned herein are incorporated by reference in their entirety. In case of a conflict between the specification and any of the incorporated references, the specification (including any amendments thereof, which may be based on an incorporated reference), shall control. Standard art-accepted meanings of terms are used herein unless indicated otherwise. Standard abbreviations for various terms are used herein.

The above discussed, and many other features and attendant advantages of the present inventions will become better understood by reference to the following detailed description of the invention.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 shows key enzymes for the biosynthesis of CBGA, CBGVA and derivatives. FPPS: farnesyl pyrophosphate synthase-Erg20, PT: prenyl transferase, ACC: acetyl-CoA carboxylase; PKS: polyketide synthase; PKC: polyketide cyclase; HCS: acyl-CoA synthase (including hexanoyl- and butyryl-); TS: terpene synthase. Enzymes of MVA pathway: Erg10: acetyl-CoA acyltranferase; Erg13: hydroxymethyl-glutaryl-CoA synthase; HMGR: Hydroxymethyl-glutaryl reductase; Erg12: mevalonate kinase; Erg8: mevalonate-5-phosphate kinase; Erg19: mevalonate diphosphate decarboxylase; MPD: phosphomevalonate decarboxylase; IPK: isopentyl phosphate kinase; IDI1: isopentyl diphosphate delta isomerase.

FIG. 2 shows the FARM region in ERG20 as well as a sequence alignment between ERG20 variants, wherein the A28 variant (2) has deletions at what would be amino acid positions equivalent to the amino acid positions 88 and 90 of the reference sequence (1).

### DETAILED DESCRIPTION OF THE INVENTION

### Some Definitions

“Identity” or “homology” refers to the extent to which the sequence of two or more nucleic acids or polypeptides is the same. In some embodiments, percent identity or homology between a sequence of interest and a second sequence over a window of evaluation, e.g., over the length of the sequence of interest, may be computed by aligning the sequences, determining the number of residues (nucleotides or amino acids) within the window of evaluation that are opposite an identical residue allowing the introduction of gaps to maximize identity, dividing by the total number of residues of the sequence of interest or the second sequence (whichever is greater) that fall within the window, and multiplying by 100. When computing the number of identical residues needed to achieve a particular percent identity or homology, fractions are to be rounded to the nearest whole number. Percent identity or homology can be calculated with the use of a variety of computer programs known in the art. For example, computer programs such as BLAST2, BLASTN, BLASTP, Gapped BLAST, etc., generate alignments and provide percent identity between sequences of interest. The algorithm of Karlin and Altschul (Karlin and Altschul, Proc. Natl. Acad. Sci. USA 87:22264-2268, 1990) modified as in Karlin and Altschul, Proc. Natl. Acad. Sci. USA 90:5873-5877, 1993 is incorporated into the NBLAST and XBLAST programs of Altschul et al. (Altschul, et al., J. Mol. Biol. 215:403-410, 1990). To obtain gapped alignments for comparison purposes, Gapped BLAST is utilized as described in Altschul et al. (Altschul, et al. Nucleic Acids Res. 25:3389-3402, 1997). When utilizing BLAST and Gapped BLAST programs, the default parameters of the respective programs may be used. A PAM250 or BLOSUM62 matrix may be used. Software for performing BLAST analyses is publicly available through the National Center for Biotechnology Information (NCBI). See the Web site having URL ncbi.nlm.nih.gov for these programs. In a specific embodiment, percent identity or homology is calculated using BLAST2 with default parameters as provided by the NCBI.

The term “homolog” is intended to mean a nucleic acid sequence which possesses close sequence identity to the nucleic acid sequence of a recited gene and wherein both nucleic acid sequences are determined to be derived from the same ancestral gene, such as through speciation, either through phylogenetic analysis or through statistical analysis of the alignment between the sequences. When making the determination that two nucleic acids sequences are homologs through statistical analysis of the alignment between the sequences, tools which are widely known and available online, such as BLAST, may be utilized to make this determination. For purposes of this definition, alignments in BLAST given an expected value (E-value) of lower than 1×10-2, will be considered sufficient for determining that both nucleic acids derived from the same ancestral gene. The term “homolog” may also similarly be used to identify two amino acid sequences which possess close sequence homology, structure and/or function and which are similarly determined to be encoded by and derived from the same ancestral gene. An “ortholog” is defined similarly as “homolog”, with the difference being the nucleic acid sequence which possesses close sequence identity to the nucleic acid sequence of a recited gene are both determined to be derived from the same ancestral gene through speciation.

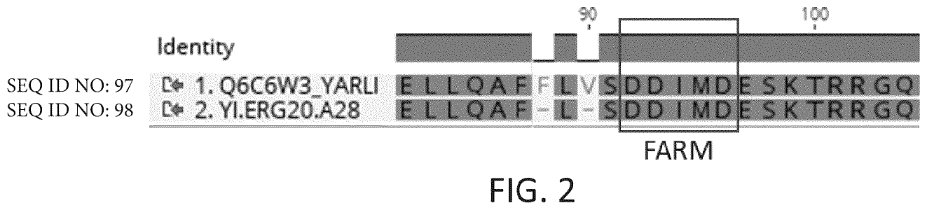

The term “equivalent”, when used to describe an amino acid position in a polypeptide sequence, means an amino acid position of a polypeptide sequence which aligns with an amino acid position of a reference polypeptide sequence when the two sequences are aligned by sequence or structural alignment techniques known in the art. When the phrase “equivalent amino acid position” is used to describe the location of a deletion, it will be evident to one of ordinary skill in the art, that an already deleted equivalent amino acid in the equivalent position can be identified by aligning the amino acids surrounding the equivalent amino acid position with the amino acids surrounding this position on the reference sequence and noting the absence of an amino acid in the compared sequence at the equivalent amino acid position (See for Example FIG. 2). Such methods for determining equivalent amino acid positions are equally applicable to wild-type proteins, mutant proteins as well as homologs and orthologs thereof.

The terms “decreased”, “reduced”, “reduction”, “decrease”, and “inhibit” are all used herein generally to mean a decrease by a statistically significant amount. However, for avoidance of doubt, “reduced”, “reduction” or “decrease” or “inhibit” means a decrease by at least 10% as compared to a reference level, for example a decrease by at least about 20%, or at least about 30%, or at least about 40%, or at least about 50%, or at least about 60%, or at least about 70%, or at least about 80%, or at least about 90% or up to and including a 100% decrease (i.e. absent level as compared to a reference sample), or any decrease between 10-100% as compared to a reference level.

The terms “increased”, “increase”, “enhance” or “activate” are all used herein to generally mean an increase by a statically significant amount; for the avoidance of any doubt, the terms “increased”, “increase”, “enhance” or “activate” means an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level.

The term “statistically significant” or “significantly” refers to statistical significance and generally means a two-standard deviation (2SD) below normal, or lower, concentration of the marker. The term refers to statistical evidence that there is a difference. It is defined as the probability of making a decision to reject the null hypothesis when the null hypothesis is actually true. The decision is often made using the p-value.

### Aims

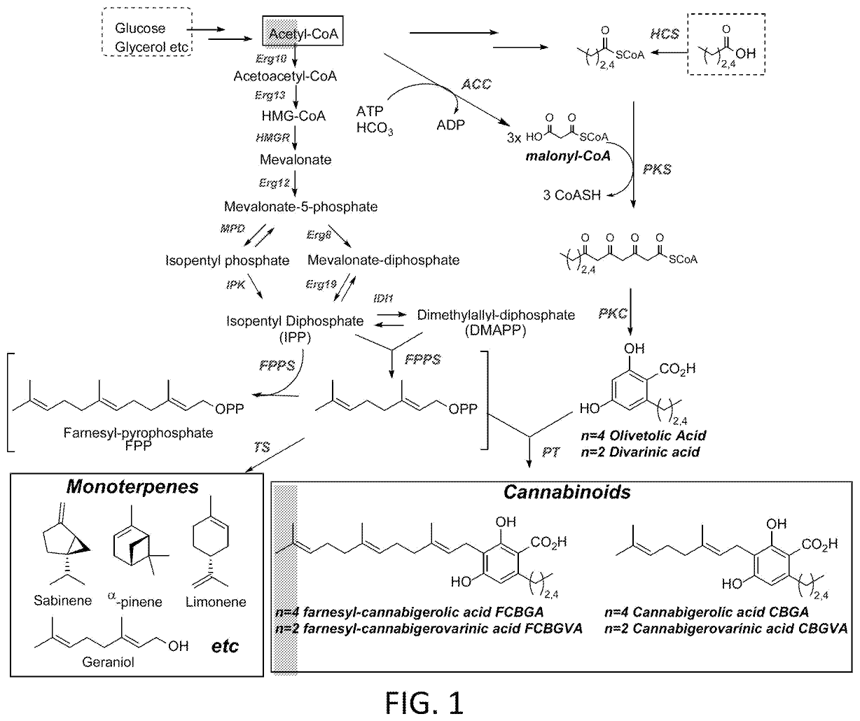

Applicants herein aim to improve the production of chemicals or other biomolecules that are produced using enzymes that utilize GPP or DMAPP as a substrate [de Bruijn et al Trends Biotechnol 2020, 38 (8), 917-934; Chen, X, et al, Pharm Biol. 2014, 52 (5), 655-660]. Examples of such chemicals are terpenoids, where a terpenoid synthase uses GPP as a substrate. Some examples of terpenoid compounds that can be made from GPP are described in the literature and include geraniol, limonene, sabinene, pinene etc. (FIG. 1 and Table 1 in Zebec et al. (2016) Curr Opin Chem Biol, 34:37-43 and FIG. 1 in Leferink, N H H et al (2019) Sci Rep 9, 11936). Another example of biomolecules derived using GPP are cannabinoids such as CBGA, CBGVA and their derivatives.

Applicants herein also aim to improve the ratio, GPP to FPP, of producing prenylated molecules-Applicants have demonstrated that ERG20.A28, in combination with expression with a GPPS (i.e. ERG20WW) and inactivation of the native ERG20, can improve CBGA production while drastically reducing FCBGA production. This can increase the overall production of the desired molecule (e.g., CBGA or CBGVA) and/or reduce undesirable by-products (e.g., FCBGA or FCBGVA) that may be difficult to separate during purification from the target molecule.

Applicants herein further aim to improve the yields and titers of compounds requiring GPP as the prenyl donor. These include prenylation with GPP of OA, DVA and other olivetol derivatives, as well as prenylation of other compounds. Some examples are described in deBruijn W J C et al (2020) Trends Biotechnol. 38 (8), 917-934

Finally, Applicants herein aim to improve formation of Acyl-CoA's [e.g., acetyl-CoA, malonyl-CoA, butryl-CoA, and hexanoyl-CoA] which are important for making GPP, OA and DVA, and all cannabinoids derived from them.

### Cells Expressing a Mutant Farnesyl Pyrophosphate Synthase Protein (FPPS)

Some aspects of the present disclosure are related to a cell producing an increased ratio of geranyl diphosphate (GPP) to farnesyl diphosphate (FPP) as compared to a control cell (e.g. having wild-type farnesyl pyrophosphate synthase protein (FPPS)), wherein the cell expresses a mutant FPPS. In some embodiments, the cell has an elevated level of GPP as compared to a control cell.

In some embodiments, the ratio of GPP to FPP, compared to control cells carrying wild-type FPPS, is increased by at least about 10%, 15%, 20%, 25%, 30%, 35%, 40%, 45%, 50%, 55%, 60%, 65%, 70%, 75%, 80%, 85%, 90%, 95%, or more. In some embodiments, the ratio of GPP to FPP, compared to control cells carrying wild-type FPPS, is increased by at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more. In some embodiments, the ratio of GPP to FPP is between 10:1 to 1:10; 4:1 to 1:4, 3:1 to 1:3, 2:1 to 1:2, 1.5:1 to 1:1.5, 1.4:1 to 1:1.4, 1.3:1 to 1:1.3, 1.2:1 to 1:1.2, or 1.1:1 to 1:1.1.

In some embodiments, the ratio of GPP to FPP is determined by measuring the ratio of CBGA to FCBGA produced by the cell.

In some embodiments, the cell has an elevated level of GPP as compared to a control cell. In some embodiments, the level of GPP compared to control cells carrying wild-type FPPS, is increased by at least about 10%, 15%, 20%, 25%, 30%, 35%, 40%, 45%, 50%, 55%, 60%, 65%, 70%, 75%, 80%, 85%, 90%, 95%, or more. In some embodiments, the level of GPP compared to control cells carrying wild-type FPPS, is increased by at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more. In some embodiments, the mutant FPPS is a mutant ERG20 with at least one insertion, deletion, or substitution (i.e., amino acid modification) at a position selected from positions 88-90 of wild-type ERG20 (SEQ ID NO: 1). In some embodiments, the mutant FPPS is a mutant ERG20 with an insertion, deletion, or substitution (i.e., amino acid modifications) at two positions selected from positions 88-90 of wild-type ERG20 (SEQ ID NO: 1). In some embodiments, the mutant FPPS is a mutant ERG20 with an insertion, deletion, or substitution (i.e., amino acid modification) at each of positions 88-90 of wild-type ERG20 (SEQ ID NO: 1).

Amino acid modifications may be amino acid substitutions, amino acid deletions and/or amino acid insertions. Amino acid substitutions may be conservative amino acid substitutions or non-conservative amino acid substitutions. A conservative replacement (also called a conservative mutation, a conservative substitution or a conservative variation) is an amino acid replacement in a protein that changes a given amino acid to a different amino acid with similar biochemical properties (e.g. charge, hydrophobicity and size). As used herein, “conservative variations” refer to the replacement of an amino acid residue by another, biologically similar residue. Examples of conservative variations include the substitution of one hydrophobic residue such as isoleucine, valine, leucine or methionine for another; or the substitution of one polar residue for another, such as the substitution of arginine for lysine, glutamic for aspartic acids, or glutamine for asparagine, and the like. Other illustrative examples of conservative substitutions include the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine, glutamine, or glutamate; methionine to leucine or isoleucine; phenylalanine to tyrosine, leucine or methionine; serine to threonine; threonine to serine; tryptophan to tyrosine; tyrosine to tryptophan or phenylalanine; valine to isoleucine or leucine, and the like.

In some embodiments, the mutant ERG20 comprises an amino acid sequence with at least 70%, 75%, 80%, 85%, 90%, 95%, 99%, 99.5%, 99.9%, or 100% identity to ERG20.A28 (e.g., SEQ ID NO: 22).

In some embodiments, the mutant ERG20 is ERG20.A28 (e.g., SEQ ID NO: 22) or a mutant ERG20 with at least about 90% homology to wild-type ERG20 (SEQ ID NO: 1) and comprising at least one insertion, deletion, or substitution at a position selected from positions 88-90 of wild-type ERG20 (SEQ ID NO: 1). In some embodiments, the mutant FPPS is a mutant ERG20 homolog or ortholog with an amino acid sequence at least about 90% homologous to the amino acid sequence of wild-type ERG20 (SEQ ID NO: 1) and comprises at least one insertion, deletion, or substitution at an amino acid position equivalent to one or more amino acid positions 88-90 of wild-type ERG20 (SEQ ID NO: 1).

In some embodiments, the mutant ERG20 has a polypeptide sequence selected from the group consisting of SEQ ID NOS: 8-31 or a sequence with at least 75%, 80%, 85%, 90%, 91%, 92%, 93%, 94%, 95%, 96%, 97%, 98%, 99%, 99.5%, 99.9%, or 99.95% identity to a polypeptide sequence selected from the group consisting of SEQ ID NOS: 8-31. In some embodiments, the mutant ERG20 is any mutant ERG20 disclosed herein.

In some embodiments, the cell has altered expression of the mutant FPPS as compared to expression of wild-type FPPS in a control cell. In some embodiments the expression of mutant FPPS is decreased by about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more as compared to expression of wild-type FPPS in a control cell. In some embodiments the expression of mutant FPPS is decreased by about 10%, 15%, 20%, 25%, 30%, 35%, 40%, 45%, 50%, 55%, 60%, 65%, 70%, 75%, 80%, 85%, 90%, 95%, or more as compared to expression of wild-type FPPS in a control cell.

In some embodiments, the cell has reduced or no expression of wild-type FPPS (e.g., Erg20). In some embodiments, the expression of wild-type FPPS is decreased by about 10%, 15%, 20%, 25%, 30%, 35%, 40%, 45%, 50%, 55%, 60%, 65%, 70%, 75%, 80%, 85%, 90%, 95%, or more as compared to a control cell expression of wild-type FPPS.

In some embodiments the FPPS or mutant FPPS is operably connected to a truncated promoter or a promoter comprising one or more insertions, deletions or substitutions (i.e., a mutant promoter). The term “promoter” as used herein refers to an expression control sequence that comprises a recognition site of a polynucleotide (DNA or RNA) to which an RNA polymerase binds. In some embodiments, the truncated or mutant promoter reduces expression in the cell of the FPPS or mutant FPPS. In some embodiments, expression with the truncated or mutant promoter is decreased by about 10%, 15%, 20%, 25%, 30%, 35%, 40%, 45%, 50%, 55%, 60%, 65%, 70%, 75%, 80%, 85%, 90%, 95%, or more as compared to a control cell expression with a wild-type promoter. In some embodiments, the promoter is truncated to about 750 bp.

In some embodiments, the cell expresses ERG20.A28 (e.g., SEQ ID NO: 22) and at least one of ERG20WW (i.e., ERG20.F88W.N119W), or a ERG20WW fused to a soluble or membrane bound prenyl transferase, including ERG20WW-MPT4.1, ERG20WW-MPT21.9, and ERG20WW-APT73.81, (e.g., soluble or membrane bound prenyl transferases as described in co-owned U.S. application No. 63/188,648, hereby incorporated herein by reference in its entirety), a GPP synthase (EC 2.5.1.1), or a farnesyl pyrophosphate synthase protein (FPPS) having a greater preference for GPP formation over FPP formation as compared to a FPPS control. Exemplary membrane bound prenyltransferases that may be fused to an ERG20 enzyme include those proteins with prenyltransferase activity with amino acid sequences comprising at least 80%, at least 90%, at least 95%, at least 97%, or at least 99% identity with the amino acid sequences of SEQ ID NOs: 47, 48 and 53-75. Exemplary membrane aromatic soluble prenyltransferases that may be fused to an ERG20 enzyme include those proteins comprising prenyltransferase activity with amino acid sequences comprising at least 80%, at least 90%, at least 95%, at least 97%, or at least 99% identity with the amino acid sequences of SEQ ID NOs: 49 and 76-78. In some embodiments, the ERG20 enzyme may be fused to a membrane-bound or soluble prentyltransferase through a linker that comprise an amino acid sequence with at least 90% identity to the amino acid sequences of SEQ ID NOs: 79-96. In some embodiments, the cell expresses ERG20.A28 (e.g., SEQ ID NO: 22) and at least one of ERG20WW (i.e., ERG20.F88W.N119W), or a ERG20WW fused to a soluble or membrane bound prenyltransferase, including ERG20WW-MPT4.1, ERG20WW-MPT21.9, ERG20WW-APT73.81, (e.g., as described in co-owned U.S. application No. 63/188,648, hereby incorporated by reference in its entirety), a GPP synthase (EC 2.5.1.1), or a farnesyl pyrophosphate synthase protein (FPPS) having a greater preference for GGPP formation over FFPP formation as compared to a FPPS control. In some embodiments, the cell does not express native ERG20.

In some embodiments, the cell has increased flux through the mevalonate (MVA) pathway as compared to a control cell. In some embodiments, flux is increased by at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more as compared to flux in a control cell.

In some embodiments, the cell having increased flux through the MVA pathway over-expresses one or more native MVA pathway genes and/or expresses one or more transgenic MVA pathway genes. In some embodiments, the cell having increased flux through the MVA pathway over-expresses one or more native MVA pathway genes and/or expresses one or more transgenic MVA pathway genes by at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more as compared to a control cell. In some embodiments, the cell having increased flux through the MVA pathway also expresses ERG20.A28.

In some embodiments, the transgenic MVA pathway genes are selected from feedback insensitive Erg13 (HMG-COA synthase, e.g., SEQ ID NO: 3 or 4), Erg12 (mevalonate kinase, e.g., SEQ ID NO: 5 or 6), mvaE (acetyl-CoA acetyltransferase/HMG-COA reductase (NADPH), e.g., SEQ ID NO: 46), and NADH-dependent HMG-COA reductase (e.g., UniProt #'s A9HWZ9 and A9BQX8). In some embodiments, the feedback insensitive Erg13 has at least 75%, 80%, 85%, 90%, 91%, 92%, 93%, 94%, 95%, 96%, 97%, 98%, 99%, 99.5%, 99.9%, or 99.95% identity to SEQ ID NO: 3 or 4. In some embodiments, the Erg12 has at least 75%, 80%, 85%, 90%, 91%, 92%, 93%, 94%, 95%, 96%, 97%, 98%, 99%, 99.5%, 99.9%, or 99.95% identity to SEQ ID NO: 5 or 6. In some embodiments, the mvaE has at least 75%, 80%, 85%, 90%, 91%, 92%, 93%, 94%, 95%, 96%, 97%, 98%, 99%, 99.5%, 99.9%, or 99.95% identity to SEQ ID NO: 46.

In some embodiments, the cell overexpresses mevalonate-5-phosphate decarboxylase (MPD, EC 4.1.1.99) and isopentenyl phosphokinase (IPK, EC 2.7.4.26). In some embodiments, the cell expresses at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more mevalonate-5-phosphate decarboxylase (MPD, EC 4.1.1.99) and isopentenyl phosphokinase (IPK, EC 2.7.4.26) as compared to a control wild-type cell.

In some embodiments, the cell overexpresses NADPH-dependent hydroxymeythylglutaryl-CoA reductase. In some embodiments, the cell expresses at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more NADPH-dependent hydroxymeythylglutaryl-CoA reductase as compared to a control wild-type cell. In some embodiments, the NADPH-dependent hydroxymeythylglutaryl-CoA reductase is a transgenic NADPH-dependent hydroxymeythylglutaryl-CoA reductase.

In some embodiments, the cell expresses one or more transgenic genes selected from limonene monoterpene synthase (e.g., PfLS from Perilla frutescens), myrcene monoterpene synthase (e.g., QiMyrS from Quercus ilex) and cineole monoterpene synthase (e.g., SfCinS1 from Salvia 15ruticose). In some embodiments, the cell has increased production of one or more monoterpenes as compared to a control wild-type cell. In some embodiments, the cell produces at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more monoterpenes as compared to a control wild-type cell.

In some embodiments, the cell has an elevated level of DMAPP or GPP as compared to a control cell. In some embodiments, the cell has at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more DMAPP or GPP as compared to a control wild-type cell. In some embodiments, the cell produces an elevated amount of one or more compounds prenylated with DMAPP as a donor, as compared to a control. In some embodiments, the cell produces at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more of one or more compounds prenylated with DMAPP as a donor, as compared to a control wild-type cell.

In some embodiments, the cell overexpresses acetyl-CoA synthase (ACS) or overexpresses both ACS and acetyl-CoA carboxylase (ACC) as compared to a control cell. In some embodiments, the cell expresses at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more ACS as compared to a control wild-type cell. In some embodiments, the cell expresses at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more ACS and ACC as compared to a control wild-type cell.

In some embodiments, the cell produces more of a cannabinoid as compared to a control wild-type cell. In some embodiments, the cell produces CBGA, CBGVA, THCA or THCVA and has increased OA or DVA production and/or CBGA or CBGVA production as compared to a control cell. In some embodiments, the cell produces at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more OA or DVA as compared to a control wild-type cell. In some embodiments, the cell produces at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more CBGA or CBGVA as compared to a control wild-type cell.

In some embodiments, the ACS is a mutant ACS with greater specificity for converting hexanoic acid to hexanoyl-CoA than the corresponding wild-type ACS. In some embodiments, the mutant ACS has about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more specificity for converting hexanoic acid to hexanoyl-coA than the corresponding wild-type ACS. In some embodiments, the ACS is selected from the group consisting of ACS1 (SEQ ID NO: 041), ACS1.1 (SEQ ID NO: 7), and an ACS with 90% homology to ACS1.1. In some embodiments, the mutant ACS has at least 75%, 80%, 85%, 90%, 91%, 92%, 93%, 94%, 95%, 96%, 97%, 98%, 99%, 99.5%, 99.9%, or 99.95% identity to SEQ ID NO: 7.

In some embodiments, the cell overexpresses pyruvate decarboxylase (PDC) and/or aldehyde dehydrogenase (ALD) as compared to a control cell. In some embodiments, the cell produces at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more pyruvate decarboxylase (PDC) and/or aldehyde dehydrogenase (ALD) as compared to a control wild-type cell.

In some embodiments, the cell overexpresses one or more non-oxidative glycolysis pathway genes, such as PTA (phosphotransacetylase, E.C. 2.3.1.8, e.g., SEQ ID NO: 42) or XPK (xylulose phosphoketolase, E.C. 4.1.2.9, e.g., SEQ ID NO: 43), and has increased cannabinoid production as compared to a control cell. In some embodiments, the cell overexpressing one or more non-oxidative glycolysis pathway genes produces at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more of a cannabinoid as compared to a control wild-type cell.

In some embodiments, the cell expresses transgenic acetylating aldehyde dehydrogenase (ADA, E.C. 1.2.1.10) and has increased cannabinoid production as compared to a control cell. In some embodiments, the cell expressing transgenic acetylating aldehyde dehydrogenase produces at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more of a cannabinoid as compared to a control wild-type cell.

In some embodiments, cannabinoids may include, but are not limited to, cannabichromene (CBC) type (e.g. cannabichromenic acid), cannabigerol (CBG) type (e.g. cannabigerolic acid), cannabidiol (CBD) type (e.g. cannabidiolic acid), Δ⁹-trans-tetrahydrocannabinol (Δ⁹-THC) type (e.g. Δ⁹-tetrahydrocannabinolic acid), Δ⁸-trans-tetrahydrocannabinol (Δ⁸-THC) type, cannabicyclol (CBL) type, cannabielsoin (CBE) type, cannabinol (CBN) type, cannabinodiol (CBND) type, cannabitriol (CBT) type, cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CBD), cannabidiol monomethylether (CBDM), cannabidiol-C₄ (CBD-C₄), cannabidivarinic acid (CBDVA), cannabidivarin (CBDV), cannabidiorcol (CBD-C₁), Δ⁹-tetrahydrocannabinolic acid A (THCA-A), Δ⁹-tetrahydrocannabinolic acid B (THCA-B), Δ⁹-tetrahydrocannabinol (THC), Δ⁹-tetrahydrocannabinolic acid-C₄ (THCA-C₄), Δ⁹-tetrahydrocannabinol-C₄ (THC-C₄), Δ⁹-tetrahydrocannabivarinic acid (THCVA), Δ⁹-tetrahydrocannabivarin (THCV), Δ⁹-tetrahydrocannabiorcolic acid (THCA-C₁), Δ⁹-tetrahydrocannabiorcol (THC-C₁), Δ⁷-cis-iso-tetrahydrocannabivarin, Δ⁸-tetrahydrocannabinolic acid (Δ⁸-THCA), Δ⁸-tetrahydrocannabinol (Δ⁸-THC), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabielsoinic acid, cannabicitranic acid, cannabinolic acid (CBNA), cannabinol (CBN), cannabinol methylether (CBNM), cannabinol-C₄, (CBN-C₄), cannabivarin (CBV), cannabinol-C₂ (CNB-C₂), cannabiorcol (CBN-C₁), cannabinodiol (CBND), cannabinodivarin (CBVD), cannabitriol (CBT), 10-ethyoxy-9-hydroxy-delta-6a-tetrahydrocannabinol, 8,9-dihydroxyl-delta-6a-tetrahydrocannabinol, cannabitriolvarin (CBTVE), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-HHCV), cannabiripsol (CBR), and trihydroxy-delta-9-tetrahydrocannabinol (triOH-THC).

In some embodiments, the cell is a yeast cell, algae cell, or a bacterial cell (e.g., Escherichia coli). In some embodiments, the yeast is an oleaginous yeast. In some embodiments, the yeast cell is a Yarrowia strain (e.g., a Yarrowia lipolytica strain), Saccharomyces strain cell, or Pichia strain cell.

Some aspects of the present disclosure are directed to a method of producing CBGA, CBGVA, or a cannabinoid derived from CBGA or CBGVA, a monoterpene (for example limonene, myrcene, cineole, etc.—see Zebec Z et al, Curr Opin Chem Biol 2016, 34, 37-43), or a monoterpenoid comprising culturing a cell as disclosed herein with a suitable carbon source under suitable conditions to produce the CBGA, monoterpene, or monoterpenoid. In some embodiments, the method further comprises isolating the CBGA, monoterpene, or monoterpenoid from the culture.

### Mutant ERG20

Some aspects of the present disclosure are directed to a mutant ERG20 (e.g., having farnesyl pyrophosphate synthase activity) with at least about 75%, 80%, 85%, 90%, 91%, 92%, 93%, 94%, 95%, 96%, 97%, 98%, 99%, 99.5%, 99.9%, or 99.95% homology or identity to wild-type ERG20 (SEQ ID NO: 1) and comprising at least one insertion, deletion, or substitution at a position selected from positions 88-90 of wild-type ERG20 (SEQ ID NO: 1). In some embodiments, the mutant ERG20 has at least about 90% homology or identity to wild-type ERG20 (SEQ ID NO: 1) and comprises at least one insertion, deletion, or substitution at a position selected from positions 88-90 of wild-type ERG20 (SEQ ID NO: 1). In some embodiments, the mutant FPPS is a mutant ERG20 homolog or ortholog with an amino acid sequence at least about 90% homologous to the amino acid sequence of wild-type ERG20 (SEQ ID NO: 1) and comprises at least one insertion, deletion, or substitution at an amino acid position equivalent to one or more amino acid positions 88-90 of wild-type ERG20 (SEQ ID NO: 1). In some embodiments, the mutant ERG20 has a polypeptide sequence selected from the group consisting of SEQ ID NOS: 8-31 or a sequence with at least 95% identity to a polypeptide sequence selected from the group consisting of SEQ ID NOS: 8-31. In some embodiments, the mutant ERG20 has a polypeptide sequence with at least 75%, 80%, 85%, 90%, 91%, 92%, 93%, 94%, 95%, 96%, 97%, 98%, 99%, 99.5%, 99.9%, or 99.95% identity to a polypeptide sequence selected from the group consisting of SEQ ID NOS: 8-31.

In some embodiments, the ERG20 comprises a substitution, deletion, or insertion at position 189 of SEQ ID NO: 1. In some embodiments, the ERG20 does not comprise a substitution, deletion, or insertion at positions 88 and 119 of SEQ ID NO: 1. In some embodiments, the ERG20 does not consist of ERG20 of SEQ ID NO: 1 with a substitution, deletion, or insertion at positions 88 and 119.

In some embodiments, the mutant ERG20 preferentially produces GPP over FPP. In some embodiments the mutant ERG20 preferentially produces about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more GPP than FPP. In some embodiments the production of GPP is increased by about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more as compared to production of GPP in a control cell.

In some embodiments, the mutant ERG20 has increased or decreased farnesyl pyrophosphate synthase activity as compared to wild-type ERG20 (e.g., SEQ ID NO: 1). In some embodiments, the activity of the mutant ERG20 is at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more higher than the activity of wild-type ERG20. In some embodiments, the activity of the mutant ERG20 is at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more lower than the activity of wild-type ERG20.

### Cells Overexpressing ACS or Both ACS and ACC

Some aspects of the present disclosure are directed to a cell overexpressing acetyl-CoA synthase (ACS, E.C. 6.2.1.1) or overexpressing both ACS and acetyl-CoA carboxylase (ACC, E.C. 6.4.1.2) as compared to a control cell. In some embodiments, the cell overexpresses acetyl-CoA synthase (ACS) or overexpresses both ACS and acetyl-CoA carboxylase (ACC) as compared to a control cell. In some embodiments, the cell expresses at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more ACS as compared to a control wild-type cell. In some embodiments, the cell expresses at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more ACS and ACC as compared to a control wild-type cell.

In some embodiments, the cell produces CBGA, CBDA, CBCA or THCA and has increased OA production and/or CBGA production as compared to a control cell. In some embodiments, the cell produces at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more OA as compared to a control wild-type cell. In some embodiments, the cell produces at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more CBGA as compared to a control wild-type cell.

In some embodiments, the cell produces CBGVA, CBDVA, CBCVA or THCVA and has increased DVA production and/or CBGVA production as compared to a control cell. In some embodiments, the cell produces at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more DVA as compared to a control cell. In some embodiments, the cell produces at least about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more CBGVA as compared to a control cell.

In some embodiments, the ACS is a mutant ACS with greater specificity for converting hexanoic acid to hexanoyl-coA than the corresponding wild-type ACS. In some embodiments, the mutant ACS has about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more specificity for converting hexanoic acid to hexanoyl-coA than the corresponding wild-type ACS. In some embodiments, the ACS is selected from the group consisting of ACS1 (SEQ ID NO: 41), ACS1.1 (SEQ ID NO: 7), and an ACS with 90% homology to ACS1.1. In some embodiments, the mutant ACS has at least 75%, 80%, 85%, 90%, 91%, 92%, 93%, 94%, 95%, 96%, 97%, 98%, 99%, 99.5%, 99.9%, or 99.95% identity to ACS1.1 (SEQ ID NO: 7).

In some embodiments, the cell is a yeast cell or a bacterial cell. In some embodiments, the yeast cell is a Yarrowia strain, Saccharomyces strain, or Pichia strain.

### Mutant ACS

Some aspects of the present disclosure are directed to a mutant acetyl-CoA synthase (ACS) (e.g., having acetyl-CoA synthase activity) selected from ACS1.1 (SEQ ID NO: 7) or an ACS with 75%, 80%, 85%, 90%, 91%, 92%, 93%, 94%, 95%, 96%, 97%, 98%, 99%, 99.5%, 99.9%, or 99.95% identity or homology to ACS1.1. In some embodiments, the mutant ACS has at least about 90% homology to ACS1.1.

In some embodiments, the mutant ACS has greater specificity for converting hexanoic acid to hexanoyl-CoA than the corresponding wild-type ACS. In some embodiments, the mutant ACS has about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more specificity for converting hexanoic acid to hexanoyl-CoA than the corresponding wild-type ACS.

### Cells Overexpressing PDC and ALD

Some aspects of the present disclosure are directed to a cell (e.g., a yeast cell or a bacterial cell, a Yarrowia strain cell, Saccharomyces strain cell, or Pichia strain cell) expressing or overexpressing pyruvate decarboxylase (PDC) and/or aldehyde dehydrogenase (ALD) as compared to a control cell. In some embodiments, the cell expresses about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more pyruvate decarboxylase (PDC) and/or aldehyde dehydrogenase (ALD) as compared to a control cell.

### Cells Overexpressing Non-Oxidative Glycolysis Pathway Genes

Some aspects of the present disclosure are directed to a cell (e.g., a yeast cell or a bacterial cell, a Yarrowia strain cell, Saccharomyces strain cell, or Pichia strain cell) expressing or overexpressing one or more non-oxidative glycolysis pathway genes, e.g., PTA (phosphotransacetylase, E.C. 2.3.1.8, e.g., SEQ ID NO: 42) or XPK (xylulose phosphoketolase, E.C. 4.1.2.9, e.g., SEQ ID NO: 43), and having increased cannabinoid production as compared to a control cell. In some embodiments, the cell expresses about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more of one or more non-oxidative glycolysis pathway genes as compared to a control cell.

### Cells Overexpressing ADA

Some aspects of the present disclosure are directed to a cell (e.g., a yeast cell or a bacterial cell, a Yarrowia strain cell, Saccharomyces strain cell, or Pichia strain cell) expressing or overexpressing transgenic acetylating aldehyde dehydrogenase (ADA, E.C. 1.2.1.10) and having increased cannabinoid production as compared to a control cell. In some embodiments, the cell expresses about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more transgenic acetylating aldehyde dehydrogenase as compared to expression of acetylating aldehyde dehydrogenase in a control cell.

### Cells Overexpressing Various Genes for Cannabinoid Production

Some aspects of the present disclosure are directed to a cell (e.g., a yeast cell or a bacterial cell, a Yarrowia strain cell, Saccharomyces strain cell, or Pichia strain cell) expressing or overexpressing one or more of a polyketide synthase, a polyketide cyclase, and a prenyl transferase. In some embodiments, the cell expresses about 1.1-fold, 1.2-fold, 1.3-fold, 1.4-fold, 1.5-fold, 1.6-fold, 1.7-fold, 1.8-fold, 1.9-fold, 2-fold, 2.5-fold, 5-fold, 10-fold, or more transgenic polyketide synthase, a polyketide cyclase, or a prenyl transferase dehydrogenase as compared to expression of the same in a control cell.

Specific examples of certain aspects of the inventions disclosed herein are set forth below in the Examples.

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention provides all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. It is contemplated that all embodiments described herein are applicable to all different aspects of the invention where appropriate. It is also contemplated that any of the embodiments or aspects can be freely combined with one or more other such embodiments or aspects whenever appropriate. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification. For example, any one or more nucleic acids, polypeptides, cells, species or types of organism, disorders, subjects, or combinations thereof, can be excluded.

Where the claims or description relate to a composition of matter, e.g., a nucleic acid, polypeptide, or cell, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., it is to be understood that methods of making compositions useful for performing the method, and products produced according to the method, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that unless otherwise indicated or evident from the context, any product or composition described herein may be considered “isolated”.

### EXAMPLES

### Introduction and Summary of Examples

The ERG20 protein is a (2E,6E)-farnesyl diphosphate synthase that has both dimethylallyltranstransferase and geranyltranstransferase activities providing the cell with FPP and GPP, which are important molecule for various essential and non-essential cellular functions. Most ERG20 proteins preferentially produce FPP relative to GPP. However, certain ERG20 mutants, like ERG20.F88W.N119W, are specific for making GPP, but likely cannot be the sole ERG20 in the cell as it would not provide sufficient levels of FPP for the cell to perform certain essential functions like ergosterol biosynthesis. Co-expressing the ERG20.F88W.N119W allele and the wild type (WT) ERG20 enables growth and improves GPP formation, but there is still considerable FPP produced.

A novel ERG20 mutant (herein referred to as ERG20.A28) was identified. When co-expressed in Yarrowia lipolytica with ERG20.F88W.N119W, it no longer requires the expression of WT ERG20 for growth. Interestingly, such a strain, expressing ERG20.A28 and ERG20.F88W.N119W and lacking WT ERG20, has significantly improved GPP production. The ERG20.A28 allele has an F88L mutation and deletion of L89 and V90. Applicants have shown herein that in CBGA producing strains, a combination of 1.) overexpressing ERG20.F88W.N119W, 2.) expressing ERG20 A28, and 3.) inactivating the native ERG20; results in substantially more CBGA produced while simultaneously reducing FCBGA, showing the utility of this invention.

Overexpression of ERG20.F88W.N119W can be replaced with other GPP synthases, especially those that are highly specific for GPP or GGPP (GPPS, EC 2.5.1.1., e.g., AgGPPS_truncated (SEQ ID NO:36) or CgGGPPS (SEQ ID NO:37)).

Besides OA derivatives, this invention is expected to improve prenylation with GPP of various compounds and natural products. Some examples of GPP prenylated natural compounds are described in deBruijn W J C et al (2020) Trends Biotechnol. 38 (8), 917-934

This invention results in increased GPP levels and thus follows that there would similarly be higher production of the GPP precursor, DMAPP, resulting in a strain with improved ability to produce compound that are prenylated with DMAPP (see de Bruijn et al Trends Biotechnol 2020, 38 (8), 917-934)

### Acyl-CoA Production is Important for Making Cannabinoids.

Applicants have shown that overexpression of the native acetyl-CoA synthase (ACS) alone and in combination with acetyl-CoA carboxylase (ACC) improved both OA production and CBGA production in cells engineered to produce CBGA or THCA.

Applicants have shown that the native ACS1 can convert acetate to acetyl-coA and hexanoic acid to hexanoyl-coA for improved cannabinoid production.

Applicants have developed an ACS mutant herein referred to as ACS1.1 that is more specific for converting hexanoic acid to hexanoyl-coA

Applicants have shown are testing that overexpression of the native pyruvate decarboxylase (PDC) and aldehyde dehydrogenase (ALD), in cells overexpressing ACC and ACS further improved both OA production and CBGA production in cells engineered to produce cannabinoids.

Applicants are testing alternative ways to produce acetyl-CoA such as non-oxidative glycolysis and acetylating aldehyde dehydrogenases.

The enzymes and strains engineered in this disclosure are the base for building industrial processes for making products that utilize GPP and acyl-CoA's during biosynthesis. In addition to cannabinoids, the inventions can be used for producing monoterpenes and other molecules that are biosynthesized using an enzyme that uses GPP as a substrate. Monoterpenes have various applications spanning from drugs, flavoring, fragrances, biofuels, and cleaning agents. To examine the use of these enzymes and strains for monoterpene production, the monoterpene synthase gene for the production of limonene, myrcene and cineole will be expressed in these strains and the production of these compounds are assessed.

This invention provides novel approaches to increase flux to GPP and increases the production of GPP relative to FPP. Furthermore, it does so in a fashion that does not result in a cell that is an auxotroph (e.g. cells lacking ERG20 can be maintained if the media is supplemented with Ergosterol or similar molecules). This is useful for producing molecules that are biosynthesized with enzymes that utilize GPP as a substrate. Lastly, it provides novel ways to improve the cellular production of acyl-CoA's which are useful molecules for the cellular production of cannabinoids. The paragraphs below support the importance of these benefits.

GPP (geranyl pyrophosphate) and FPP (farnesyl pyrophosphate) are prenyl compounds produced in cells by farnesyl pyrophosphate synthetase (FPPS), that in certain organisms is designated ERG20. ERG20 is a bifunctional enzyme that first catalyzes the condensation of dimethylallyl pyrophosphate (DMAPP) and isopentenyl pyrophosphate (IPP) to produce geranyl pyrophosphate (GPP) followed by the condensation of GPP and IPP to produce farnesyl pyrophosphate (FPP) (FIG. 1). Mutations in this enzyme, such as the F96W/N127W in S. cerevisiae ERG20 (analogous to F88W/N119W in Yl.Erg20), have been identified to significantly reduce this second step (GPP to FPP) such that the mutant enzyme primarily produces GPP (Ignea C, et al ACS Synth Biol. 2014, 3, 298-306). In addition, copy number of ERG20 was halved by deleting one copy in a diploid yeast. This manipulation significantly improved GPP-derived sabinene production in S. cerevisiae (Ignea C, et al ACS Synth Biol. 2014, 3, 298-306). However, as FPP is essential for cell viability, a wild-type ERG20 is maintained in the cells to allow for FPP production. FPP is essential for cell viability as it is a precursor for ergosterol—a key plasma membrane component.

To minimize the production of FCBGA, production of FPP needs to be reduced while keeping enough FPP for cell viability. In the disclosed strains, a mutated form of ERG20, designated as ERG20WW containing the F88W & N119W mutations that preferably produces GPP is expressed. To test if this mutated Erg20 is still able to produce sufficient FPP for cell viability and reduce FPP production, Applicants attempted to disrupt the native ERG20 using CRISPR/CAS9. This attempt resulted in no clones with a complete inactivation (marker gene integrated within the coding sequence) of the native ERG20; however, several clones did exhibit a reduction in the production of FCBGA. Interestingly, these clones also had an increase in CBGA production.

In one clone, SB565.A28, the ERG20 gene was amplified and sequenced. The result indicated a 6 base in-frame deletion at the CRISPR/CAS9 cut site. This deletion results in an F88L mutation and deletion of L89 and V90. Applicants speculate that this mutation results in an ERG20 with decreased activity. The genome was also sequenced using a MinION (Oxford Nanopore Technologies) and confirmed the presence of the deletion in ERG20 and the lack of the wild type sequence. Applicants reference this allele of ERG20 as ERG20.A28.

In a second clone, SB565.B32, the genome was again sequenced using a MinION (Oxford Nanopore Technologies) and it was found that the wild type gene was still present, but the promoter was truncated to ˜750 bp. This shortened promoter likely resulted in a significant reduction in expression and thus overall activity, resulting in reduced FPP production.

To test the effect of the ERG20.A28 allele, this mutated version of ERG20, expressed with a ˜750 bp ERG20 promoter, was introduced into Yarrowia expressing ERG20WW and the ERG20WW-tMPT4 fusion (this fusion has shown to improve activity and selectivity of prenyltransferase and has been described in co-owned U.S. Provisional Application No. 63/188,648, hereby incorporated by reference in its entirety). The endogenous ERG20 was then disrupted and the resulting strain examined for CBGA & FCBGA production from OA. These engineered clones resulted in significant increase in CBGA and decrease in FCBGA.

In addition to production of CBGA and other cannabinoids, GPP can be used for the production of various monoterpenes/monoterpenoids (see Zebec Z et al, Curr Opin Chem Biol 2016, 34, 37-43). These compounds can have various applications spanning drugs, flavoring, fragrances, biofuels and cleaning agents. This strain carrying the ERG20.A28 allele can be used as a host strain for the production of these monoterpene/monoterpenoid compounds. To examine this possibility, the monoterpene synthase gene for limonene, myrcene and cineole is introduced into a base strain carrying the ERG20.A28 allele, disrupted for the native ERG20 and overexpressing the ERG20WW allele, and the production of these compounds are assessed.

In addition to the ERG20.A28 allele, alternative mutations in ERG20 could reduce its activity in producing FPP (possibly in part by producing GPP). Mutations in S. cerevisiae ERG20 at K197 (K189 in Yl.ERG20) may also result in mutations with reduced activity (DOI 10.1002/bit.23129). Characterization of these alternative mutants as well as in combination with the ERG20.A28 allele may be of interest.

This region just upstream of the FARM region in ERG20 is well conserved in other FPPS proteins. It is well conserved in S. cerevisiae (Sc) ERG20. A similar mutation in S. cerevisiae in concert with overexpression of ScERG20WW allele and inactivation of the wild-type ScERG20 may also result in increased flux to GPP.

Lowering the FPP selectivity of Saccharomyces Erg20 has been achieved by mutating two different amino acids F96 and N127 (Ignea C, et al ACS Synth Biol. 2014, 3, 298-306). It has been shown that equivalent positions in the Yarrowia are conserved (F88 and N119) and their mutagenesis also improve production of linalool (monoterpene derived from GPP) formation (Cao X, et al. Bior Tech 2017, 245, 1641-1644). In the current work, Applicants discovered that an F88L mutation and deletion of L89 and V90 creates an enzyme that Applicants believe has decreased activity, and only produces enough FPP to support growth. This area of the protein is clearly important for the enzyme's activity and selectivity so further mutagenesis at these positions may further improve GPP production in our strain. See, e.g., FIG. 2.

Overexpression of the native ACC and ACS should increase flux to acetyl-CoA and malonyl-CoA which can improve CBGA titers by increasing flux to OA and/or GPP. In agreement with this, Applicants have shown that in cultures containing cells supplemented with hexanoic acid, overexpression of ACC and ACS improved both the titers of OA and CBGA. Though ACC and ACS overexpression have been used to improve the production of various compounds derived from acetyl-CoA and malonyl-CoA with mixed results, the Applicants have not seen any reports demonstrating improved production of cannabinoids based on overexpression of ACS and/or ACC.

Another aspect of this invention is to increase the flux to GPP using the mevalonate pathway (MVA) in a strain described above (expressing ERG20.A28 & ERG20.F88W.N119W and lacking WT ERG20) or other Yarrowia or yeast strains. A detailed description of the biosynthesis pathways to all common cannabinoids is shown in FIG. 1. All cells biosynthesize GPP, so an optimized cell would be modified to upregulate this pathway. Various examples of engineered microbial strains (E. coli, yeast, Yarrowia, etc.) with upregulated mevalonate pathway (MVA) have been published (A A Malico, M A Calzini, A K Gayen, G J Williams J Ind Microbiol Biotechnol 2020, 47, 675-702). It is noteworthy that although most of the enzymes of the MVA pathway have been altered, i.e. over-expressed, mutated, or replaced with enzymes from other organisms, no universal solution that upregulate equally GPP or FPP biosynthesis can be applied to all cells and derived products, although some general rules for its upregulation have been identified (Y. Zu, K L J Prather, G. Stephanopoulos Curr Opin Biotechnol, 2020, 66, 1-8).

To increase the flux to the MVA pathway, all or a subset of the MVA pathway enzymes will be overexpressed. These enzymes will include the native Yarrowia enzymes as well as selected heterologous genes with reduced or no substrate/product regulation and inhibition. For example, hydroxymeythylglutaryl-CoA synthase (Erg13; EC 2.3.3.10), in most organisms including yeast is inhibited by substrates (acetoacetyl-CoA) products (HMG-COA) and various acyl-CoAs including hexanoyl-CoA (Middleton, B.; Biochem. J. 1972, 126, 35-47). Similarly, mevalonate kinase (Erg12, EC 2.7.1.36) is inhibited by GPP and FPP (Fu, Z et al Biochemistry, 2008, 47, 3715-3724). Feed-back insensitive enzymes for both these steps have been identified. A mutant Erg13 from Brassica juncea (BjErg13_mut) with high activity and reduced product inhibition has been described (Nagegowda D A et al Biochem J, 2004, 383, 517-527) while a very active mutant of Enterococcus faecalis (EfErg13_mut) will also be used (Steussy, C N et al Biochemistry, 2006, 45, 14407-14414). For Erg12, enzymes without any inhibition have been described from various methanogenic archaea including Methanosarcina mazei (Erg12_Q8PW39) and Methanosaeta concili (Erg12_F4BZB3).

Certain enzymes of the MVA pathway can catalyze both forward and reverse reactions, and as a result, overexpression will not improve flux unless a strong “pull” is present in the pathway. Such an enzyme is phosphomevalonate kinase (Erg8, EC 2.7.4.2). To improve flux, either the next enzyme of the pathway, mevalonate pyrophosphate decarboxylase or Erg19, will be overexpressed or an alternative pathway that bypasses this step will be introduced using mevalonate phosphate decarboxylase (MPD EC 4.1.1.99) and isopentenyl phosphate kinase (IPK EC 2.7.4.26) (FIG. 1). Finally, the native NADPH-dependent hydroxymeythylglutaryl-CoA reductases (HMGR) will need to be overexpressed since this enzyme has been shown to be one of the major bottlenecks of the MVA pathway in both yeast and Yarrowia. Furthermore, NADH-dependent HMGRs will also be overexpressed. Some examples of the NADH-dependent HMGRs that will be expressed including but are not limited to UniProt #'s A9HWZ9 and A9BQX8.

### Technical Description, Details and Supporting Data

### Example 1: Identification of ERG20 Mutants that Improved CBGA/FCBGA Ratio AND Improved CBGA Titers (OA>CBGA)

To improve the CBGA/FCBGA ratio in a CBGA producing strain, Applicants sought to further reduce FPP production by disrupting the native ERG20 gene. Applicants' speculated that such a disruption could be carried out in a strain that overexpresses the ERG20WW allele (mutation that results in an enzyme that primarily produces GPP) as this allele may produce enough FPP to sustain cell growth. Thus, disruption of the native ERG20 was attempted in strain SB491 that expresses ERG20WW and a ERG20WW-MPT4 fusion. ERG20 gene was targeted using CRISPR/CAS9 with gRNA targeting sequence GCAGGCGTTTTTCCTCGTGT (SEQ ID NO: 2) and DNA fragments carrying homology arms and a split hph marker. 288 Transformants were screened by junction PCRs and 32 clones that appeared to be positive for at least one of the 5′ or 3′ junctions were screened for CBGA production from OA. Clones were inoculated into 500 μL YNBD (2% Dextrose)+0.5% CAA+100 mM MES (pH6.5) in deep-well 96-well plate and incubated at 30° C. in a high speed shaker for 24 hours. 2 μL of this preculture was used to inoculate 500 μL YNBD (6% Dextrose)+0.5% CAA+100 mM MES (pH6.5)+3 mM OA in deep-well 96-well plate and incubated at 30° C. in a high speed shaker for 48 hours. The cultures were quenched with 500 μL Ethanol containing internal standard and analyzed by LC. Production of CBGA, FCBGA and the CBGA/FCBGA ratios are shown in Table 1. Of these, two clones stood out-A28 and B32. In both cases, the CBGA/FCBGA ratio is significantly improved. As is evident, this improvement is due to both increased CBGA titers and reduced FCBGA titers.

TABLE 1
CBGA and FCBGA titers of select SB565 clones fed OA.

| strain | CBGA (μM) | FCBGA (μM) | CBGA/FCBGA |
|---|---|---|---|
| SB491 | 389.8 | 77.0 | 5.1 |
| SB565_A02 | 393.6 | 85.7 | 4.6 |
| SB565_A05 | 393.7 | 84.1 | 4.7 |
| SB565_A22 | 62.7 | 12.3 | 5.1 |
| SB565_A25 | 344.4 | 78.3 | 4.4 |
| SB565_A28 | 455.0 | 14.1 | 32.2 |
| SB565_B01 | 68.8 | 13.1 | 5.3 |
| SB565_B03 | 69.7 | 13.4 | 5.2 |
| SB565_B09 | 202.4 | 27.7 | 7.3 |
| SB565_B32 | 499.6 | 21.5 | 23.2 |
| SB565_C09 | 401.7 | 80.7 | 5.0 |
| SB565_C12 | 375.7 | 80.3 | 4.7 |
| SB565_C20 | 354.3 | 79.6 | 4.4 |
| SB565_C25 | 380.4 | 75.8 | 5.0 |
| SB565_C29 | 326.3 | 78.8 | 4.1 |
| SB565_C30 | 351.7 | 89.8 | 3.9 |
| SB565_D13 | 353.6 | 71.9 | 4.9 |
| SB565_E31 | 356.1 | 51.6 | 6.9 |
| SB565_F03 | 405.0 | 77.4 | 5.2 |
| SB565_F04 | 380.2 | 79.3 | 4.8 |
| SB565_F05 | 359.9 | 78.7 | 4.6 |
| SB565_F15 | 497.3 | 57.2 | 8.7 |
| SB565_F24 | 530.2 | 59.3 | 8.9 |
| SB565_F27 | 134.0 | 43.5 | 3.1 |
| SB565_G02 | 350.7 | 75.2 | 4.7 |
| SB565_G08 | 222.4 | 101.3 | 2.2 |
| SB565_G11 | 405.6 | 76.1 | 5.3 |
| SB565_G13 | 394.1 | 74.4 | 5.3 |
| SB565_G25 | 393.6 | 90.1 | 4.4 |
| SB565_H13 | 3.9 | 71.0 | 0.1 |
| SB565_H17 | 184.5 | 84.2 | 2.2 |
| SB565_H20 | 384.8 | 91.7 | 4.2 |
| SB565_H21 | 360.3 | 75.5 | 4.8 |
| SB565_H25 | 221.9 | 50.9 | 4.4 |
| SB565_H29 | 185.5 | 85.0 | 2.2 |
| SB565_H30 | 280.3 | 42.5 | 6.6 |
| SB565_H32 | 386.0 | 79.3 | 4.9 |
| SB565_I02 | 406.3 | 79.2 | 5.1 |
| SB565_I14 | 446.2 | 94.4 | 4.7 |
| SB565_I17 | 177.4 | 75.9 | 2.3 |
| SB565_I20 | 403.6 | 78.4 | 5.1 |
| SB565_I21 | 384.7 | 74.8 | 5.1 |
| SB565_I24 | 122.1 | 36.9 | 3.3 |
| SB565_I27 | 0.0 | 0.0 | — |
| SB565_I31 | 368.2 | 79.1 | 4.7 |

Molecular diagnostics of these clones via qPCR, Sanger sequencing of PCR products and ONT (Oxford Nanopore Technologies) sequencing identified that the A28 clone did not have a wild type ERG20 sequence but the ERG20 gene carried a 6 base (TCCTCG) deletion at the gRNA cut site. This deletion affected three codons (for FLV at position 88-90) resulting in a single codon coding for Leu. Applicants refer to this allele of ERG20 as ERG20.A28 (SEQ ID NO: 22). Similar set of diagnostics of clone B32 showed that this clone had a wild type ERG20 coding sequence but a shortened ˜750 bp promoter. The results herein clearly show that reduction of the native Erg20 activity in Yarrowia together with expressing a synthase that preferably produces GPP (like ERG20WW) increase both the GPP flux as measured by the increased titers of CBGA and the GPP to FPP ratio as measured by the CBGA/FCBA ratio of products.

### Example 2: Disruption of Native ERG20 in OA to CBGA Strain Expressing ERG20.A28 Allele Results in Improved CBGA/FCBGA Ratio AND Improved CBGA Titers. (OA to CBGA)

To confirm that the improved CBGA/FCBGA ratio and increased titers of CBGA was due to the 6 base deletion in ERG20.A28, this allele was first cloned behind a 750 bp ERG20 promoter and introduced into SB491 (a strain that contains ERG20WW and ERG20WW-MPT4 and can convert OA to CBGA) to generate strain SB748. Then the native wild type ERG20 was disrupted to generate SB751. SB491, SB748 and 11 clones of SB751 were examined for CBGA and FCBGA production from OA as described in Example 1. The results are shown in Table 2. As is evident, this genetic manipulation resulted in improved CBGA/FCBGA ratio and a significant increase in CBGA titers.

TABLE 2

| name | CBGA (μM) | FCBGA (μM) | CBGA/FCBGA |
|---|---|---|---|
| SB491 | 377.4 | 94.0 | 4.0 |
| SB748 | 340.2 | 105.9 | 3.2 |
| SB751_01 | 1064.7 | 72.1 | 14.8 |
| SB751_02 | 1397.5 | 94.4 | 14.8 |
| SB751_03 | 1364.6 | 92.3 | 14.8 |
| SB751_04 | 1169.6 | 77.5 | 15.1 |
| SB751_05 | 1072.4 | 70.5 | 15.2 |
| SB751_06 | 1204.4 | 79.5 | 15.1 |
| SB751_07 | 1233.8 | 82.2 | 15.0 |
| SB751_08 | 1460.5 | 97.5 | 15.0 |
| SB751_09 | 1578.6 | 103.8 | 15.2 |
| SB751_10 | 1114.3 | 69.6 | 16.0 |
| SB751_11 | 1137.1 | 75.7 | 15.0 |

### Example 3: Disruption of Native ERG20 in Hexanoic/Butyric Acid to CBGA/CBGVA Strain Expressing ERG20.A28 Allele Results in Improved CBG(V)A/FCBG(V)A Ratio AND Improved CBG(V)A Titers

To examine the effects of the ERG20.A28 allele under the 750 bp ERG20 promoter, in combination with overexpression of ERG20WW and disruption of the native ERG20 gene, on the production of CBGA/CBGVA from hexanoic/butyric acid, these engineering steps were introduced into the CBG(V)A producing strain, SB1268 (expresses HCS2, PKS1, PKC1.1, ERG20ww, ERG20-PKC1.1-MPT4). First, the ERG20.A28 allele under the 750 bp ERG20 promoter was introduced, then the native ERG20 was disrupted. The resulting strain, SB1554, was compared to SB1268 in small scale fermentation using either hexanoic acid or butyric acid as feed. These strains were inoculated into 500 μL YNBD (6% Dextrose)+1% CAA+100 mM MES (pH6.5) in a deep-well 96-well plate and incubated at 30° C. in high speed shaker for 24 hours. 2 μL of this preculture was used to inoculate 500 μL YNBD (6% Dextrose)+1% CAA+100 mM MES (pH6.5)+2.5 mM hexanoic acid or butyric acid in deep-well 96-well plate and incubated at 30° C. in a high speed shaker for 24 hours. 25 μL of a 100 mM hexanoic acid solution in 100 mM MES (6.5) or 25 μL of a 100 mM butyric acid solution in 100 mM MES (6.5) was added and the plate returned to the high speed shaker for an additional 24 hours. The cultures were quenched with 500 μL Ethanol containing internal standard and analyzed by LC. The results from the hexanoic acid feed are shown in Table 3A. As is evident, this genetic manipulation resulted in increased CBGA titers and in improved CBGA/FCBGA ratio. The results from the butyric acid feed are shown in Table 3B. As is evident, this genetic manipulation resulted in significant improvement in CBGVA titers. In this experiment, as no FCBGA was detected CBGVA/FCBGVA ratios was not evaluated.

TABLE 3A
CBGA and FCBGA and CBGA/FCBGA ratios for
hexanoic acid fed fermentations of SB1268 and its A28
derivative, SB 155. Results are reported in μM.

| | CBGA | FCBGA | CBGA/FCBGA |
|---|---|---|---|
| SB1268 | 627.9 ± | 54.3 ± 1.6 | 11.6 ± 0.1 |
| | 19.2 | | |
| SB1554 | 922.5 ± | 29.5 ± 1.3 | 31.3 ± 1.7 |
| | 21.5 | | |

TABLE 3B
CBGVA and FCBGVA for butyric acid fed fermentations
of SB1268 and its A28 derivative, SB1554. Results are reported in μM.

| | CBGVA | FCBGVA* |
|---|---|---|
| SB1268 | 179.6 ± 7.1 | nd |
| SB1554 | 500.1 ± 17.1 | nd |

*FCBGVA was not detected (nd).

### Example 4: Alter Expression of ERG20.A28 by Adjusting Promoter Length

To assess if expression level of ERG20.A28 has an effect on the improved CBG(V)A titers and CBG(V)A/FCBG(V)A ratio, a set of plasmids are constructed with different lengths of the ERG20 promoter. These expression cassettes are introduced into SB491 to generate strains expressing ERG20.A28. The native ERG20 is disrupted in these strains and the resulting strain assessed for CBG(V)A production based on OA/DVA feeds.

### Example 5: Further Improve MVA Pathway Flux by Overexpression of Pathway Genes

To determine if further upregulating the MVA pathway would benefit CBG(V)A production, plasmids for the expression of genes (HMG1 (SEQ ID NO: 50), tHMG1 (amino acids 2-495 removed, SEQ ID NO: 51), Enterococcus faecalis mvaE or IDI1 (SEQ ID NO: 52)) to upregulated MVA pathway flux were introduced into SB1085 (expresses HCS2, ERG20WW, ERG20WW-MPT4, ERG20.A28 and disrupted for ERG20; derived from SB751). The resulting strains were assayed for production of CBGVA based on DVA feed. Clones were inoculated into 500 μL YNBD (2% Dextrose)+1% CAA+100 mM MES (pH6.5) in deep-well 96-well plate and incubated at 30° C. in a high speed shaker for 24 hours. 2 μL of this preculture was used to inoculate 500 μL YNBD (6% Dextrose)+1% CAA+100 mM MES (pH6.5)+2 mM DVA in deep-well 96-well plate and incubated at 30° C. in a high speed shaker for 48 hours. The cultures were quenched with 500 μL Ethanol containing internal standard and analyzed by LC. As shown in Table 4, addition of HMG1, tHMG1, mvaE, or IDI1 significantly improved CBGVA production.

TABLE 4
CBGVA production from DVA in SB1085 transformed with
HMG1, tHMGR1, mvaE or IDI1. Results are reported in μM.

| | Gene | |
|---|---|---|
| Plasmid | overexpressed | CBGVA |
| — | — | 906.0 ± 10.5 |
| pCL-SE-0441 | HMG1 | 1467.7 ± 36.0 |
| pCL-SE-0442 | tHMG1 | 1641.2 ± 83.2 |
| pCL-SE-0446 | mvaE | 1524.0 ± 31.1 |
| pCL-SE-0501 | IDI1 | 1114.4 ± 20.0 |

### Example 6: Mutagenesis of Erg20 and Further Testing

Mutant libraries will be prepared as shown in Table 5. These libraries will be screened for improved GPP formation in Yarrowia. Selected mutants will be expressed in E. coli purified and their activities will be identified.

TABLE 5
Mutant libraries to screen for improved GPP formation.

| Library | F88 | L89 | V90 |
|---|---|---|---|
| Lib_1 | Deletion | SSM | deletion |
| Lib_2 | Deletion | SSM | SSM |
| Lib_3 | Deletion | Deletion | SSM |
| Lib_4 | SSM | SSM | Deletion |
| Lib_5 | SSM | Deletion | SSM |
| Lib_6 | SSM | Deletion | Deletion |

### Example 7: Monoterpene (Limonene) Production in Strains Expressing ERG20.A28 Allele, Disrupted for Native ERG20 and Overexpressing ERG20WW Allele

To examine if the increased flux to GPP can be used to increase production of monoterpenes (diverse set of compounds derived from GPP that have uses in pharmaceuticals, cosmetic, agriculture and food industries), as proof of concept, the monoterpene synthases for limonene (PfLS from Perilla frutescens) was introduced into SB809 (strain expressing ERG20.A28, disrupted for ERG20 and overexpressing ERG20WW) as well as SB491 (wild type ERG20 control) resulting ins strains SB1027 and SB1030, respectively. The production of the monoterpene was examined in these strains.

Strains transformed with an expression cassette for PfLS, and untransformed parent strains, were examined for limonene production by culturing in YPD (8%)+100 mM MES (pH6.5)+10% dodecane overlay for 72 hours. Samples were prepared by mixing with an equal volume of heptane containing methyl nonadecanoate (CAS 1731-94-8) as internal standard. The samples were analyzed by GC-FID and the results are shown in Table below. As can be seen from the results shown in Table 6, the A28 engineered strain was able to produce ˜4× the amount of limonene compared to the ERG20 wild type strain.

TABLE 6
Limonene production in A28 strains

| Strain | Description | Limonene (mg/L) |
|---|---|---|
| SB491 | Erg20 WT parent | 0.0 |
| SB809 | Erg20.A28, Δerg20 parent | 0.0 |
| SB1030 | Erg20 WT with PfLS | 7.5 |
| SB1027 | Erg20.A28, Δerg20 with PfLS | 31.8 |

### Example 8: Expression of ACS1 and ACC1 Improve Cannabinoid Production

pCL-SE-0709 expresses the ACS1 and ACC1 genes each from the UAS1B(4×)pTEF1intron promoter. This vector was linearized and transformed into SB-691 which is a strain that can produce CBGA with hexanoic acid supplementation. Clones (SB888_01 to 07) from this transformation that expressed ACS1 and ACC1 produced more OA and CBGA compared to the parental strain when supplemented with hexanoic acid (Table 7)

TABLE 7
OA and CBGA produced with overexpression of ACS1
and ACC1.

| Strain | OA (μM) | CBGA (μM) |
|---|---|---|
| SB888_01 | 689 | 248 |
| SB888_03 | 843 | 281 |
| SB888_04 | 791 | 274 |
| SB888_05 | 747 | 272 |
| SB888_06 | 802 | 248 |
| SB888_07 | 757 | 251 |
| SB691_13 | 493 | 156 |

### Example 9: ACS1.0 can Activate Hexanoic Acid

As overexpression of ACS1.0 improves OA and CBGA production from hexanoic acid feeds (Table 7), we thought ACS1.0 may have the ability to activate hexanoic acid to hexanoyl-CoA. To assess if ACS1.0 has this hexanoyl-CoA synthase activity, ACS1.0 was introduced into strain sCL137 that has no HCS but carries PKS1 and PKC1.1 for OA and OL production to generate SB999. HCS2 was also introduced to generate SB998 as control. Clones of each was assayed for OA and OL production by hexanoic acid feed as described in Example 3. Results (Table 8) show that like HCS (SB998), ACS1.0 is able to increase OA and OL production in SB999 compared to sCL137. This indicates that ACS1.0 is able to activate hexanoic acid.

TABLE 8
OA, OL and OA + OL in strains with introduction of ACS1.0
to activate hexanoic acid to hexanoyl-CoA.

| | Gene | | | |
|---|---|---|---|---|
| | added | OA | OL | OA + OL |
| SB998 | HCS2 | 334.5 ± 7.7 | 237.5 ± 3.8 | 572.1 ± 11.5 |
| SB999 | ACS1.0 | 299.1 ± 45.8 | 162.3 ± 16.9 | 461.5 ± 62.4 |
| sCL137 | — | 168.1 ± 3.5 | 96.6 ± 2.5 | 264.7 ± 6.0 |

### Example 10: ACS1.1 Shows Improved Specificity to Hexanoic Acid

As overexpression of ACS1.0 exhibits the ability to activate hexanoic acid to hexanoyl-CoA, we examined if introduction of homologous mutations found in HCS2 would improve specificity of ACS for hexanoic acid. This mutant, ACS1.1 was introduced into strain sCL137 that has no HCS but carries PKS1 and PKC1.1 for OA and OL production to generate SB1000. SB998 (HCS2), SB999 (ACS1.0) and the parent strain, sCL137, were used as controls. Clones of each was assayed for OA and OL production by hexanoic acid feed as described in Example 3. Results (Table 9) show that ACS1.1 (SB1000) is able to increase OA and OL production similar to HCS (SB998) and with higher titers compared to ACS1.0 (SB999). These results indicates that ACS1.1 is able to activate hexanoic acid with improved activity compared to ACS1.0.

TABLE 9
OA, OL and OA + OL in strains with introduction of ACS1.1
to activate hexanoic acid to hexanoyl-CoA.

| | Gene | | | |
|---|---|---|---|---|
| | added | OA | OL | OA + OL |
| SB998 | HCS2 | 334.5 ± 7.7 | 237.5 ± 3.8 | 572.1 ± 11.5 |
| SB999 | ACS1.0 | 299.1 ± 45.8 | 162.3 ± 16.9 | 461.5 ± 62.4 |
| SB1000 | ACS1.1 | 331.8 ± 67.0 | 233.9 ± 48.5 | 565.7 ± 108.5 |
| sCL137 | — | 168.1 ± 3.5 | 96.6 ± 2.5 | 264.7 ± 6.0 |

### Example 11: Over-Expression of PDC5 and ALD5 Improves Cannabinoid Production

PDC5 and ALD5 genes will be cloned into a vector that provides their expression from the UAS1B(4×)pTEF1intron promoter. This vector will be linearized and transformed into SB-691 which is a strain that can produce CBGA with hexanoic acid supplementation. Clones from this transformation that expressed ALD5 and PDC5 will be screened for OA and CBGA production compared to the parental strain when supplemented with hexanoic acid. The combination of PDC5 and ALD5 will increase flux from pyruvate to acetate, the latter is a substrate that can be converted to acetyl-CoA for making cannabinoids.

### Example 12: Expression of Non-Oxidative Glycolysis Pathway Genes Improve Cannabinoid Production

Acetyl-CoA formation will be increased for producing cannabinoids by re-wiring carbon central metabolism and to increase flux through the pentose phosphate pathway (PPP). Phosphofructokinase (Pfk) will be deleted to block glycolysis and heterologous phosphoketolase (Xpk) and phosphotransacetylase (Pta) will be expressed to convert the PPP intermediate xylulose-5-P to acetyl-CoA. These deletions and overexpression's will be made in a strain like SB-691 which is a strain that can produce CBGA with hexanoic acid supplementation. Clones will be screened for OA and CBGA production compared to the parental strain when supplemented with hexanoic acid.

### Example 13: Expression of Acetylating Aldehyde Dehydrogenases Improve Cannabinoid Production

Acylating aldehyde dehydrogenase encoding genes will be cloned into a vector that provides their expression from the UAS1B(4×)pTEF1intron promoter. This vector will be linearized and transformed into SB-691 which is a strain that can produce CBGA with hexanoic acid supplementation. Clones from this transformation that express acylating aldehyde dehydrogenase's will be screened for OA and CBGA production compared to the parental strain when supplemented with hexanoic acid. The acylating aldehyde dehydrogenases will increase flux to hexanoyl-CoA, the latter is a substrate that can be used for making cannabinoids.

TABLE 10
Strain list:

| Strain | Key gene(s) expressed | ERG20 disrupted? |
|---|---|---|
| SB491 | ERG20WW, ERG20WW-MPT4 | |
| SB748 | ERG20WW, ERG20WW-MPT4, | |
| | ERG20.A28 | |
| SB751 | ERG20WW, ERG20WW-MPT4, | Δerg20 |
| | ERG20.A28 | |
| SB691 | HCS2, PKS1, PKC1.1, HMG1, | |
| | ERG20WW, ERG20WW-PKC1.1-MPT4 | |
| SB809 | ERG20WW, ERG20WW-MPT4, | Δerg20 |
| | ERG20.A28 | |
| SB888 | HCS2, PKS1, PKC1.1, HMG1, | |
| | ERG20WW, ERG20WW-PKC1.1-MPT4, | |
| | ACS1, ACC1 | |
| SB996 | HCS2, PKS1, PKC1.1, HMG1, | Δerg20 |
| | ERG20WW, ERG20WW-PKC1.1-MPT4, | |
| | ERG20.A28 | |
| SB998 | HCS2, PKS1, PKC1.1 | |
| SB999 | ACS1, PKS1, PKC1.1 | |
| SB1000 | ACS1.1, PKS1, PKC1.1 | |
| SB1027 | ERG20WW, ERG20WW-MPT4, | Δerg20 |
| | ERG20.A28, PILS | |
| SB1030 | ERG20WW, ERG20WW-MPT4, | |
| | ERG20.A28, PfLS | |
| SB1085 | HCS2, ERG20WW, ERG20WW-MPT4, | Δerg20 |
| | ERG20.A28 | |
| SB1268 | HCS2, PKS1, PKC1.1, HMG1, | |
| | ERG20WW, ERG20WW-PKC1.1-MPT4, | |
| | ACS | |
| SB1544 | HCS2, PKS1, PKC1.1, HMG1, | Δerg20 |
| | ERG20WW, ERG20WW-PKC1.1-MPT4, | |
| | ACS, ERG20.A28 | |

TABLE 11
Plasmid list:

| Plasmid | Host(s) | Key gene(s) expressed |
|---|---|---|
| pCL-SE-0441 | E. coli/Yarrowia | HMG1 |
| pCL-SE-0442 | E. coli/Yarrowia | tHMG1 (amino acids 2-495 deleted) |
| pCL-SE-0446 | E. coli/Yarrowia | IDI1 |
| pCL-SE-0501 | E. coli/Yarrowia | mvaE (Enterococcus faecalis) |
| pCL-SE-0709 | E. coli/Yarrowia | ACS1, ACC1 |
| pCL-SE-0831 | E. coli/Yarrowia | HCS2, PKS1, PKC1.1 |

### Analytical Methods:

### Cannabinoids

Cannabinoids and their intermediates were analyzed by LC-MS under the following conditions:

### Method Conditions:

Column: 2.1×50 mm Cosmocore PBr (Nacalai USA, Inc.)
Mobile Phase: A; 0.1% formic acid in water, B; 0.1% formic acid in acetonitrile
Flow Rate: 0.45 mL/min
Temperature: 50 Celsius
Injection vol.: 1 μL
Gradient: 20% B at 0 min, 70% B at 2.3 min, 89% B at 4.2 min, 20% B at 4.3 min, 20% B at 6 min
Detection: UV DAD @ 275 nm and QToF MS

### Monoterpenes

Terpenes, such as limonene, myrcene, and eucalyptol were analyzed by GC-FID under the following conditions:
Column: DB-FastFAME (Agilent G3903-63011)
Mobile Phase: Helium
Flow Rate: 1.5 mL/min
Temperature profile: 110-180° C. @ 40° C./min, 180-220° C. @10° C., 220-250° C. @30° C./min
Injection vol.: 2 μL with 50:1 split
Detection: FID @ 250° C.

Sequences:

Y1.ERG20 (WT) (SEQ ID NO: 1)
MSKAKFESVFPRISEELVQLLRDEGLPQDAVQWFSDSLQYNCV
GGKLNRGLSVVDTYQLLTGKKELDDEEYYRLALLGWLIELLQAFFLVSDDIM
DESKTRRGQPCWYLKPKVGMIAINDAFMLESGIYILLKKHFRQEKYYIDLVEL
FHDISFKTELGQLVDLLTAPEDEVDLNRFSLDKHSFIVRYKTAYYSFYLPVVL
AMYVAGITNPKDLQQAMDVLIPLGEYFQVQDDYLDNFGDPEFIGKIGTDIQD
NKCSWLVNKALQKATPEQRQILEDNYGVKDKSKELVIKKLYDDMKIEQDYL
DYEEEVVGDIKKKIEQVDESRGFKKEVLNAFLAKIYKRQK*
>gRNA (SEQ ID NO: 2)
GCAGGCGTTTTTCCTCGTGT
>BjErg13_mut (SEQ ID NO: 3)
MAKNVGILAMDIYFPPTCVQQEALEAHDGASKGKYTIGLGQD
CLAFCTELEDVISMSFNAVTSLLEKYKIDPKQIGRLEVGSETVIDKSKSIKTFLM
QLFEKCGNTDVEGVDSTNACYGGTAALLNCVNWVESNSWDGRYGLVICTDS
AVYAEGPARPTGGAAAIAMLIGPDAPIVFESKLRGSHMANVYDFYKPNLASE
YPVVDGKLSQTCYLMALDSCYKHLCNKFEKLEGKEFSINDADYFVFHSPYNK
LVQKSFARLLYNDFLRNASSIDEAAKEKFTPYSSLSLDESYQSRDLEKVSQQL
AKTYYDAKVQPTTLVPKQVGNMYTASLYAAFASLVHNKHSDLAGKRVVMF
SYGAGSTATMFSLRLCENQSPFSLSNIASVMDVGGKLKARHEYAPEKFVETM
KLMEHRYGAKEFVTSKEGILDLLAPGTYYLKEVDSLYRRFYGKKGDDGSITNGH
>EfErg13_mut (SEQ ID NO: 4)
MTIGIDKISFFVPPYYIDMTALAEARNVDPGKFHIGIGQDQMAV
NPISQDIVTFAANAAEAILTKEDKEAIDMVIVGTESSIDESKAAAVVLHRLMGI
QPFARSFEIKEGCYGATAGLQLAKNHVALHPDKKVLVVAADIAKYGLNSGG
EPTQGAGAVAMLVASEPRILALKEDNVMLTQDIYDFWRPTGHPYPMVDGPL
SNETYIQSFAQVWDEHKKRTGLDFADYDALAFHIPYTKMGKKALLAKISDQT
EAEQERILARYEESIIYSRRVGNLYTGSLYLGLISLLENATTLTAGNQIGLFSYG
SGAVAEFFTGELVAGYQNHLQKETHLALLDNRTELSIAEYEAMFAETLDTDI
DQTLEDELKYSISAINNTVRSYRN
>Erg12_Q8PW39 (SEQ ID NO: 5)
MVSCSAPGKIYLFGEHAVVYGETAIACAVELRTR VRAELNDSIT
IQSQIGRTGLDFEKHPYVSAVIEKMRKSIPINGVFLTVDSDIPVGSGLGSSAAVT
IASIGALNELFGFGLSLQEIAKLGHEIEIKVQGAASPTDTYVSTFGGVVTIPERR
KLKTPDCGIVIGDTGVFSSTKELVANVRQLRESYPDLIEPLMTSIGKISRIGEQL
VLSGDYASIGRLMNVNQGLLDALGVNILELSQLIYSARAAGAFGAKITGAGG
GGCMVALTAPEKCNQVAEAVAGAGGKVTITKPTEQGLKVD
>Erg12_F4BZB3 (SEQ ID NO: 6)
MTMASAPGKIILFGEHAVVSGTAALGGAIDLRARAIVQSLPGRI
LIETDDLSLRGFSLDLSTGEIRSASAAYATRYVSAVLKELGARDVRVMIESDIP
PAAGLGSSASIVVATVAALNGHLGLELSQKEIAALSYRIEKEVQKGRGSPMDT
ALATYGGYQRIADDNQRLDLPPLEMVVGYTRLPHDTFSLVEKVQLLKERYPD
LVGPIFQAIGAISERAAPLIREQRLKDLGELMDINHGLLEALGVGSRELSELVY
AARNTGGALGAKLTGAGGGGCMIALPGMAGKDALLVALRQARGMAFAAM
MGCEGVRLEVA
>ACS1.1 (SEQ ID NO: 7)
MSEDHPAIHPPSEFKDNHPHFGGPHLDCLQDYHQLHKESIEDPK
AFWKKMANELISWSTPFETVRSGGFEHGDVAWFPEGQLNASYNCVDRHAFA
NPDKPAIIFEADEPGQGRIVTYGELLRQVSQVAATLRSFGVQKGDTVAVYLP
MIPEAIVTLLAITRIGAVHSVIFAGFSSGSLRDRINDAKSKVVVTTDASMRGGK
TIDTKKIVDEALRDCPSVTHTLVFRRAGVENLAWTEGRDFWWHEEVVKHRP
YLAPVPVASEDPIFLLYTSGSTGTPKGLAHATGGYLLGAALTAKYVFDIHGDD
KLFTAGDVGWIGGHTYVLYGPLMLGATTVVFEGTPAYPSFSRYWDIVDDHKI
THEYVAPTALRLLKRAGTHHIKHDLSSLRTLGSAGEPIAPDVWQWYNDNIGR
GKAHICDTYGQTETGSHIIAPMAGVTPTKPGSASLPVFGIDPVIIDPVSGEELKG
NNVEGVLALRSPWPSMARTVWNTHERYMETYLRPYPGYYFTGDGAARDND
GFYWIRGRVDDVVNVSGHRLSTAEIEAALIEHAQVSESAVVGVHDDLTGQAV
NAFVALKNPVEDVDALRKELVVQVRKTIGPFAAPKNVIIVDDLPKTRSGKIMR
RILRKVLAGEEDQLGDISTLANPDVVQTIIEVVHSLKK
>Y1.ERG20.AF (SEQ ID NO: 8)
MSKAKFESVFPRISEELVQLLRDEGLPQDAVQWFSDSLQYNCV
GGKLNRGLSVVDTYQLLTGKKELDDEEYYRLALLGWLIELLQAFLVSDDIMD
ESKTRRGQPCWYLKPKVGMIAINDAFMLESGIYILLKKHFRQEKYYIDLVELF
HDISFKTELGQLVDLLTAPEDEVDLNRFSLDKHSFIVRYKTAYYSFYLPVVLA
MYVAGITNPKDLQQAMDVLIPLGEYFQVQDDYLDNFGDPEFIGKIGTDIQDN
KCSWLVNKALQKATPEQRQILEDNYGVKDKSKELVIKKLYDDMKIEQDYLD
YEEEVVGDIKKKIEQVDESRGFKKEVLNAFLAKIYKRQK*
>Y1.ERG20.AL (SEQ ID NO: 9)
MSKAKFESVFPRISEELVQLLRDEGLPQDAVQWFSDSLQYNCV
GGKLNRGLSVVDTYQLLTGKKELDDEEYYRLALLGWLIELLQAFFVSDDIMD
ESKTRRGQPCWYLKPKVGMIAINDAFMLESGIYILLKKHFRQEKYYIDLVELF
HDISFKTELGQLVDLLTAPEDEVDLNRFSLDKHSFIVRYKTAYYSFYLPVVLA
MYVAGITNPKDLQQAMDVLIPLGEYFQVQDDYLDNFGDPEFIGKIGTDIQDN
KCSWLVNKALQKATPEQRQILEDNYGVKDKSKELVIKKLYDDMKIEQDYLD
YEEEVVGDIKKKIEQVDESRGFKKEVLNAFLAKIYKRQK*
>Y1.ERG20.AV (SEQ ID NO: 10)
MSKAKFESVFPRISEELVQLLRDEGLPQDAVQWFSDSLQYNCV
GGKLNRGLSVVDTYQLLTGKKELDDEEYYRLALLGWLIELLQAFFLSDDIMD
ESKTRRGQPCWYLKPKVGMIAINDAFMLESGIYILLKKHFRQEKYYIDLVELF
HDISFKTELGQLVDLLTAPEDEVDLNRFSLDKHSFIVRYKTAYYSFYLPVVLA
MYVAGITNPKDLQQAMDVLIPLGEYFQVQDDYLDNFGDPEFIGKIGTDIQDN
KCSWLVNKALQKATPEQRQILEDNYGVKDKSKELVIKKLYDDMKIEQDYLD
YEEEVVGDIKKKIEQVDESRGFKKEVLNAFLAKIYKRQK*
>Y1.ERG20.AFLV (SEQ ID NO: 11)
MSKAKFESVFPRISEELVQLLRDEGLPQDAVQWFSDSLQYNCV
GGKLNRGLSVVDTYQLLTGKKELDDEEYYRLALLGWLIELLQAFSDDIMDES
KTRRGQPCWYLKPKVGMIAINDAFMLESGIYILLKKHFRQEKYYIDLVELFHD
ISFKTELGQLVDLLTAPEDEVDLNRFSLDKHSFIVRYKTAYYSFYLPVVLAMY
VAGITNPKDLQQAMDVLIPLGEYFQVQDDYLDNFGDPEFIGKIGTDIQDNKCS
WLVNKALQKATPEQRQILEDNYGVKDKSKELVIKKLYDDMKIEQDYLDYEE
EVVGDIKKKIEQVDESRGFKKEVLNAFLAKIYKRQK*
>Y1.ERG20.FLV > A (SEQ ID NO: 12)
MSKAKFESVFPRISEELVQLLRDEGLPQDAVQWFSDSLQYNCV
GGKLNRGLSVVDTYQLLTGKKELDDEEYYRLALLGWLIELLQAFASDDIMDE
SKTRRGQPCWYLKPKVGMIAINDAFMLESGIYILLKKHFRQEKYYIDLVELFH
DISFKTELGQLVDLLTAPEDEVDLNRFSLDKHSFIVRYKTAYYSFYLPVVLAM
YVAGITNPKDLQQAMDVLIPLGEYFQVQDDYLDNFGDPEFIGKIGTDIQDNKC
SWLVNKALQKATPEQRQILEDNYGVKDKSKELVIKKLYDDMKIEQDYLDYE
EEVVGDIKKKIEQVDESRGFKKEVLNAFLAKIYKRQK*
>Y1.ERG20.FLV > R (SEQ ID NO: 13)
MSKAKFESVFPRISEELVQLLRDEGLPQDAVQWFSDSLQYNCV
GGKLNRGLSVVDTYQLLTGKKELDDEEYYRLALLGWLIELLQAFRSDDIMDE
SKTRRGQPCWYLKPKVGMIAINDAFMLESGIYILLKKHFRQEKYYIDLVELFH
DISFKTELGQLVDLLTAPEDEVDLNRFSLDKHSFIVRYKTAYYSFYLPVVLAM
YVAGITNPKDLQQAMDVLIPLGEYFQVQDDYLDNFGDPEFIGKIGTDIQDNKC
SWLVNKALQKATPEQRQILEDNYGVKDKSKELVIKKLYDDMKIEQDYLDYE
EEVVGDIKKKIEQVDESRGFKKEVLNAFLAKIYKRQK*
>Y1.ERG20.FLV > N (SEQ ID NO: 14)
MSKAKFESVFPRISEELVQLLRDEGLPQDAVQWFSDSLQYNCV
GGKLNRGLSVVDTYQLLTGKKELDDEEYYRLALLGWLIELLQAFNSDDIMDE
SKTRRGQPCWYLKPKVGMIAINDAFMLESGIYILLKKHFRQEKYYIDLVELFH
DISFKTELGQLVDLLTAPEDEVDLNRFSLDKHSFIVRYKTAYYSFYLPVVLAM
YVAGITNPKDLQQAMDVLIPLGEYFQVQDDYLDNFGDPEFIGKIGTDIQDNKC
SWLVNKALQKATPEQRQILEDNYGVKDKSKELVIKKLYDDMKIEQDYLDYE
EEVVGDIKKKIEQVDESRGFKKEVLNAFLAKIYKRQK*
>Y1.ERG20.FLV > D (SEQ ID NO: 15)
MSKAKFESVFPRISEELVQLLRDEGLPQDAVQWFSDSLQYNCV
GGKLNRGLSVVDTYQLLTGKKELDDEEYYRLALLGWLIELLQAFDSDDIMDE
SKTRRGQPCWYLKPKVGMIAINDAFMLESGIYILLKKHFRQEKYYIDLVELFH
DISFKTELGQLVDLLTAPEDEVDLNRFSLDKHSFIVRYKTAYYSFYLPVVLAM
YVAGITNPKDLQQAMDVLIPLGEYFQVQDDYLDNFGDPEFIGKIGTDIQDNKC
SWLVNKALQKATPEQRQILEDNYGVKDKSKELVIKKLYDDMKIEQDYLDYE
EEVVGDIKKKIEQVDESRGFKKEVLNAFLAKIYKRQK*
>Y1.ERG20.FLV > C (SEQ ID NO: 16)
MSKAKFESVFPRISEELVQLLRDEGLPQDAVQWFSDSLQYNCV
GGKLNRGLSVVDTYQLLTGKKELDDEEYYRLALLGWLIELLQAFCSDDIMDE
SKTRRGQPCWYLKPKVGMIAINDAFMLESGIYILLKKHFRQEKYYIDLVELFH
DISFKTELGQLVDLLTAPEDEVDLNRFSLDKHSFIVRYKTAYYSFYLPVVLAM
YVAGITNPKDLQQAMDVLIPLGEYFQVQDDYLDNFGDPEFIGKIGTDIQDNKC
SWLVNKALQKATPEQRQILEDNYGVKDKSKELVIKKLYDDMKIEQDYLDYE
EEVVGDIKKKIEQVDESRGFKKEVLNAFLAKIYKRQK*
>Y1.ERG20.FLV > Q (SEQ ID NO: 17)
MSKAKFESVFPRISEELVQLLRDEGLPQDAVQWFSDSLQYNCV
GGKLNRGLSVVDTYQLLTGKKELDDEEYYRLALLGWLIELLQAFQSDDIMDE
SKTRRGQPCWYLKPKVGMIAINDAFMLESGIYILLKKHFRQEKYYIDLVELFH
DISFKTELGQLVDLLTAPEDEVDLNRFSLDKHSFIVRYKTAYYSFYLPVVLAM
YVAGITNPKDLQQAMDVLIPLGEYFQVQDDYLDNFGDPEFIGKIGTDIQDNKC
SWLVNKALQKATPEQRQILEDNYGVKDKSKELVIKKLYDDMKIEQDYLDYE
EEVVGDIKKKIEQVDESRGFKKEVLNAFLAKIYKRQK*
>Y1.ERG20.FLV > E (SEQ ID NO: 18)
MSKAKFESVFPRISEELVQLLRDEGLPQDAVQWFSDSLQYNCV
GGKLNRGLSVVDTYQLLTGKKELDDEEYYRLALLGWLIELLQAFESDDIMDE
SKTRRGQPCWYLKPKVGMIAINDAFMLESGIYILLKKHFRQEKYYIDLVELFH
DISFKTELGQLVDLLTAPEDEVDLNRFSLDKHSFIVRYKTAYYSFYLPVVLAM
YVAGITNPKDLQQAMDVLIPLGEYFQVQDDYLDNFGDPEFIGKIGTDIQDNKC
SWLVNKALQKATPEQRQILEDNYGVKDKSKELVIKKLYDDMKIEQDYLDYE
EEVVGDIKKKIEQVDESRGFKKEVLNAFLAKIYKRQK*
>Y1.ERG20.FLV > G (SEQ ID NO: 19)
MSKAKFESVFPRISEELVQLLRDEGLPQDAVQWFSDSLQYNCV
GGKLNRGLSVVDTYQLLTGKKELDDEEYYRLALLGWLIELLQAFGSDDIMDE
SKTRRGQPCWYLKPKVGMIAINDAFMLESGIYILLKKHFRQEKYYIDLVELFH
DISFKTELGQLVDLLTAPEDEVDLNRFSLDKHSFIVRYKTAYYSFYLPVVLAM
YVAGITNPKDLQQAMDVLIPLGEYFQVQDDYLDNFGDPEFIGKIGTDIQDNKC
SWLVNKALQKATPEQRQILEDNYGVKDKSKELVIKKLYDDMKIEQDYLDYE
EEVVGDIKKKIEQVDESRGFKKEVLNAFLAKIYKRQK*
>Y1.ERG20.FLV > H (SEQ ID NO: 20)
MSKAKFESVFPRISEELVQLLRDEGLPQDAVQWFSDSLQYNCV
GGKLNRGLSVVDTYQLLTGKKELDDEEYYRLALLGWLIELLQAFHSDDIMDE
SKTRRGQPCWYLKPKVGMIAINDAFMLESGIYILLKKHFRQEKYYIDLVELFH
DISFKTELGQLVDLLTAPEDEVDLNRFSLDKHSFIVRYKTAYYSFYLPVVLAM
YVAGITNPKDLQQAMDVLIPLGEYFQVQDDYLDNFGDPEFIGKIGTDIQDNKC
SWLVNKALQKATPEQRQILEDNYGVKDKSKELVIKKLYDDMKIEQDYLDYE
EEVVGDIKKKIEQVDESRGFKKEVLNAFLAKIYKRQK*
>Y1.ERG20.FLV > I (SEQ ID NO: 21)
MSKAKFESVFPRISEELVQLLRDEGLPQDAVQWFSDSLQYNCV
GGKLNRGLSVVDTYQLLTGKKELDDEEYYRLALLGWLIELLQAFISDDIMDE
SKTRRGQPCWYLKPKVGMIAINDAFMLESGIYILLKKHFRQEKYYIDLVELFH
DISFKTELGQLVDLLTAPEDEVDLNRFSLDKHSFIVRYKTAYYSFYLPVVLAM
YVAGITNPKDLQQAMDVLIPLGEYFQVQDDYLDNFGDPEFIGKIGTDIQDNKC
SWLVNKALQKATPEQRQILEDNYGVKDKSKELVIKKLYDDMKIEQDYLDYE
EEVVGDIKKKIEQVDESRGFKKEVLNAFLAKIYKRQK*
>Y1.ERG20.FLV > L (i.e., Y1.ERG20.A28) (SEQ ID NO: 22)
MSKAKFESVFPRISEELVQLLRDEGLPQDAVQWFSDSLQYNCV
GGKLNRGLSVVDTYQLLTGKKELDDEEYYRLALLGWLIELLQAFLSDDIMDE
SKTRRGQPCWYLKPKVGMIAINDAFMLESGIYILLKKHFRQEKYYIDLVELFH
DISFKTELGQLVDLLTAPEDEVDLNRFSLDKHSFIVRYKTAYYSFYLPVVLAM
YVAGITNPKDLQQAMDVLIPLGEYFQVQDDYLDNFGDPEFIGKIGTDIQDNKC
SWLVNKALQKATPEQRQILEDNYGVKDKSKELVIKKLYDDMKIEQDYLDYE
EEVVGDIKKKIEQVDESRGFKKEVLNAFLAKIYKRQK*
>Y1.ERG20.FLV > K (SEQ ID NO: 23)
MSKAKFESVFPRISEELVQLLRDEGLPQDAVQWFSDSLQYNCV
GGKLNRGLSVVDTYQLLTGKKELDDEEYYRLALLGWLIELLQAFKSDDIMDE
SKTRRGQPCWYLKPKVGMIAINDAFMLESGIYILLKKHFRQEKYYIDLVELFH
DISFKTELGQLVDLLTAPEDEVDLNRFSLDKHSFIVRYKTAYYSFYLPVVLAM
YVAGITNPKDLQQAMDVLIPLGEYFQVQDDYLDNFGDPEFIGKIGTDIQDNKC
SWLVNKALQKATPEQRQILEDNYGVKDKSKELVIKKLYDDMKIEQDYLDYE
EEVVGDIKKKIEQVDESRGFKKEVLNAFLAKIYKRQK*
>Y1.ERG20.FLV > M (SEQ ID NO: 24)
MSKAKFESVFPRISEELVQLLRDEGLPQDAVQWFSDSLQYNCV
GGKLNRGLSVVDTYQLLTGKKELDDEEYYRLALLGWLIELLQAFMSDDIMD
ESKTRRGQPCWYLKPKVGMIAINDAFMLESGIYILLKKHFRQEKYYIDLVELF
HDISFKTELGQLVDLLTAPEDEVDLNRFSLDKHSFIVRYKTAYYSFYLPVVLA
MYVAGITNPKDLQQAMDVLIPLGEYFQVQDDYLDNFGDPEFIGKIGTDIQDN
KCSWLVNKALQKATPEQRQILEDNYGVKDKSKELVIKKLYDDMKIEQDYLD
YEEEVVGDIKKKIEQVDESRGFKKEVLNAFLAKIYKRQK*
>Y1.ERG20.FLV > F (SEQ ID NO: 25)
MSKAKFESVFPRISEELVQLLRDEGLPQDAVQWFSDSLQYNCV
GGKLNRGLSVVDTYQLLTGKKELDDEEYYRLALLGWLIELLQAFFSDDIMDE
SKTRRGQPCWYLKPKVGMIAINDAFMLESGIYILLKKHFRQEKYYIDLVELFH
DISFKTELGQLVDLLTAPEDEVDLNRFSLDKHSFIVRYKTAYYSFYLPVVLAM
YVAGITNPKDLQQAMDVLIPLGEYFQVQDDYLDNFGDPEFIGKIGTDIQDNKC
SWLVNKALQKATPEQRQILEDNYGVKDKSKELVIKKLYDDMKIEQDYLDYE
EEVVGDIKKKIEQVDESRGFKKEVLNAFLAKIYKRQK*
>Y1.ERG20.FLV > P (SEQ ID NO: 26)
MSKAKFESVFPRISEELVQLLRDEGLPQDAVQWESDSLQYNCV
GGKLNRGLSVVDTYQLLTGKKELDDEEYYRLALLGWLIELLQAFPSDDIMDE
SKTRRGQPCWYLKPKVGMIAINDAFMLESGIYILLKKHFRQEKYYIDLVELFH
DISFKTELGQLVDLLTAPEDEVDLNRFSLDKHSFIVRYKTAYYSFYLPVVLAM
YVAGITNPKDLQQAMDVLIPLGEYFQVQDDYLDNFGDPEFIGKIGTDIQDNKC
SWLVNKALQKATPEQRQILEDNYGVKDKSKELVIKKLYDDMKIEQDYLDYE
EEVVGDIKKKIEQVDESRGFKKEVLNAFLAKIYKRQK*
>Y1.ERG20.FLV > S (SEQ ID NO: 27)
MSKAKFESVFPRISEELVQLLRDEGLPQDAVQWFSDSLQYNCV
GGKLNRGLSVVDTYQLLTGKKELDDEEYYRLALLGWLIELLQAFSSDDIMDE
SKTRRGQPCWYLKPKVGMIAINDAFMLESGIYILLKKHFRQEKYYIDLVELFH
DISFKTELGQLVDLLTAPEDEVDLNRFSLDKHSFIVRYKTAYYSFYLPVVLAM
YVAGITNPKDLQQAMDVLIPLGEYFQVQDDYLDNFGDPEFIGKIGTDIQDNKC
SWLVNKALQKATPEQRQILEDNYGVKDKSKELVIKKLYDDMKIEQDYLDYE
EEVVGDIKKKIEQVDESRGFKKEVLNAFLAKIYKRQK*
>Y1.ERG20.FLV > T (SEQ ID NO: 28)
MSKAKFESVFPRISEELVQLLRDEGLPQDAVQWFSDSLQYNCV
GGKLNRGLSVVDTYQLLTGKKELDDEEYYRLALLGWLIELLQAFTSDDIMDE
SKTRRGQPCWYLKPKVGMIAINDAFMLESGIYILLKKHFRQEKYYIDLVELFH
DISFKTELGQLVDLLTAPEDEVDLNRFSLDKHSFIVRYKTAYYSFYLPVVLAM
YVAGITNPKDLQQAMDVLIPLGEYFQVQDDYLDNFGDPEFIGKIGTDIQDNKC
SWLVNKALQKATPEQRQILEDNYGVKDKSKELVIKKLYDDMKIEQDYLDYE
EEVVGDIKKKIEQVDESRGFKKEVLNAFLAKIYKRQK*
>Y1.ERG20.FLV > W (SEQ ID NO: 29)
MSKAKFESVFPRISEELVQLLRDEGLPQDAVQWFSDSLQYNCV
GGKLNRGLSVVDTYQLLTGKKELDDEEYYRLALLGWLIELLQAFWSDDIMD
ESKTRRGQPCWYLKPKVGMIAINDAFMLESGIYILLKKHFRQEKYYIDLVELF
HDISFKTELGQLVDLLTAPEDEVDLNRFSLDKHSFIVRYKTAYYSFYLPVVLA
MYVAGITNPKDLQQAMDVLIPLGEYFQVQDDYLDNFGDPEFIGKIGTDIQDN
KCSWLVNKALQKATPEQRQILEDNYGVKDKSKELVIKKLYDDMKIEQDYLD
YEEEVVGDIKKKIEQVDESRGFKKEVLNAFLAKIYKRQK*
>Y1.ERG20.FLV > Y (SEQ ID NO: 30)
MSKAKFESVFPRISEELVQLLRDEGLPQDAVQWESDSLQYNCV
GGKLNRGLSVVDTYQLLTGKKELDDEEYYRLALLGWLIELLQAFYSDDIMDE
SKTRRGQPCWYLKPKVGMIAINDAFMLESGIYILLKKHFRQEKYYIDLVELFH
DISFKTELGQLVDLLTAPEDEVDLNRFSLDKHSFIVRYKTAYYSFYLPVVLAM
YVAGITNPKDLQQAMDVLIPLGEYFQVQDDYLDNFGDPEFIGKIGTDIQDNKC
SWLVNKALQKATPEQRQILEDNYGVKDKSKELVIKKLYDDMKIEQDYLDYE
EEVVGDIKKKIEQVDESRGFKKEVLNAFLAKIYKRQK*
>Y1.ERG20.FLV > V (SEQ ID NO: 31)
MSKAKFESVFPRISEELVQLLRDEGLPQDAVQWESDSLQYNCV
GGKLNRGLSVVDTYQLLTGKKELDDEEYYRLALLGWLIELLQAFVSDDIMDE
SKTRRGQPCWYLKPKVGMIAINDAFMLESGIYILLKKHFRQEKYYIDLVELFH
DISFKTELGQLVDLLTAPEDEVDLNRFSLDKHSFIVRYKTAYYSFYLPVVLAM
YVAGITNPKDLQQAMDVLIPLGEYFQVQDDYLDNFGDPEFIGKIGTDIQDNKC
SWLVNKALQKATPEQRQILEDNYGVKDKSKELVIKKLYDDMKIEQDYLDYE
EEVVGDIKKKIEQVDESRGFKKEVLNAFLAKIYKRQK*
>GPS1.1 (SEQ ID NO: 32)
MSKAKFESVFPRISEELVQLLRDEGLPQDAVQWFSDSLQYNCV
GGKLNRGLSVVDTYQLLTGKKELDDEEYYRLALLGWLIELLQAFWLVSDDI
MDESKTRRGQPCWYLKPKVGMIAIWDAFMLESGIYILLKKHFRQEKYYIDLV
ELFHDISFKTELGQLVDLLTAPEDEVDLNRFSLDKHSFIVRYKTAYYSFYLPVV
LAMYVAGITNPKDLQQAMDVLIPLGEYFQVQDDYLDNFGDPEFIGKIGTDIQ
DNKCSWLVNKALQKATPEQRQILEDNYGVKDKSKELVIKKLYDDMKIEQDY
LDYEEEVVGDIKKKIEQVDESRGFKKEVLNAFLAKIYKRQK
>GPS1.1-L11-MPT4.1(SEQ ID NO: 33)
MSKAKFESVFPRISEELVQLLRDEGLPQDAVQWFSDSLQYNCV
GGKLNRGLSVVDTYQLLTGKKELDDEEYYRLALLGWLIELLQAFWLVSDDI
MDESKTRRGQPCWYLKPKVGMIAIWDAFMLESGIYILLKKHFRQEKYYIDLV
ELFHDISFKTELGQLVDLLTAPEDEVDLNRFSLDKHSFIVRYKTAYYSFYLPVV
LAMYVAGITNPKDLQQAMDVLIPLGEYFQVQDDYLDNFGDPEFIGKIGTDIQ
DNKCSWLVNKALQKATPEQRQILEDNYGVKDKSKELVIKKLYDDMKIEQDY
LDYEEEVVGDIKKKIEQVDESRGFKKEVLNAFLAKIYKRQKGGAEAAAKEAA
AKAGGSGGGSGGGGSGGSGGGGSGGGGSMSDNSIATKILNFGHTCWKLQRP
YAVKGMISIACGLFGRELFNNRHLFSWGLMWKAFFALVPILSFNFFAAIMNQI
YDVDIDRINKPDLPLVSGEMSIETAWILSIIVALTGLIVTIKLKSAPLFVFIYIFGI
FAGFAYSVPPIRWKQYPFTNFLITISSHVGLAFTSYSATTSALGLPFVWRPAFSF
IIAFMTVMGMTIAFAKDISDIEGDAKYGVSTVATKLGARNMTFVVSGVLLLN
YLVSISIGIIWPQVFKSNIMILSHAILAFCLIFQTRELALANYASAPSRQFFEFIW
LLYYAEYFVYVFI
>GPS1.1-L11-MPT21.9 (SEQ ID NO: 34)
MSKAKFESVFPRISEELVQLLRDEGLPQDAVQWFSDSLQYNCV
GGKLNRGLSVVDTYQLLTGKKELDDEEYYRLALLGWLIELLQAFWLVSDDI
MDESKTRRGQPCWYLKPKVGMIAIWDAFMLESGIYILLKKHFRQEKYYIDLV
ELFHDISFKTELGQLVDLLTAPEDEVDLNRFSLDKHSFIVRYKTAYYSFYLPVV
LAMYVAGITNPKDLQQAMDVLIPLGEYFQVQDDYLDNFGDPEFIGKIGTDIQ
DNKCSWLVNKALQKATPEQRQILEDNYGVKDKSKELVIKKLYDDMKIEQDY
LDYEEEVVGDIKKKIEQVDESRGFKKEVLNAFLAKIYKRQKGGAEAAAKEAA
AKAGGSGGGSGGGGSGGSGGGGSGGGGSMSDNSIATKILNFGHTCWKLQRP
YVVKGMISIACGLFGKELLHNTNLISWGLMWKAFFALVPILSENFFASIMNQI
YDVDIDRINKPDLPLVSGEMSIETAWILSIIVALTGLIVTIKLKSAPLFVFIYIFGI
FAGFAYSVPPIRWKQNPSTNFLITISSHVGLAFTSYYASRAALGLPFELRPSFTF
LLAFMTVMGMTIAFAKDISDIEGDAKYGVSTVATKLGARNMTFVVSGVLLL
NYVAAILAGIIWPQAFNSNVMLLSHAILAFCLIFQTRELALANYASAPSRQFFE
FIWLLYYAEYFVYVFI
>GPS1.1-L13-APT73.81 (SEQ ID NO: 35)
MSKAKFESVFPRISEELVQLLRDEGLPQDAVQWFSDSLQYNCV
GGKLNRGLSVVDTYQLLTGKKELDDEEYYRLALLGWLIELLQAFWLVSDDI
MDESKTRRGQPCWYLKPKVGMIAIWDAFMLESGIYILLKKHFRQEKYYIDLV
ELFHDISFKTELGQLVDLLTAPEDEVDLNRFSLDKHSFIVRYKTAYYSFYLPVV
LAMYVAGITNPKDLQQAMDVLIPLGEYFQVQDDYLDNFGDPEFIGKIGTDIQ
DNKCSWLVNKALQKATPEQRQILEDNYGVKDKSKELVIKKLYDDMKIEQDY
LDYEEEVVGDIKKKIEQVDESRGFKKEVLNAFLAKIYKRQKGGSGSAGSAAG
SGEFGGMDEVYAAVEQTSRLLDVPCSPDRFEPVWKAFGDQLPDSHLVFSMA
AGEAHRGELDFDFSLRPEGADPYTTALEHGFIEPTDHPVGSVLAEVGKRFAIA
SYGVEYGVVGGFKKSYAFFPLDDFPPLAQFAEVPSVPPCLAGHVETLTRLGFD
DKVSIIGVNYRKNTLNVYLAASAVDTGDKLALLRAFGYPEPDARVRQFIERSF
RLYPTFNWDSSAAERICFAVHTQQPGELPAPHDEPTEAFARQVPHVYEGGREF
VSGVALAPSGASYYKLAALYQKGRRCLD
>AgGPPS2_truncated (SEQ ID NO: 36)
MQLLNPPQKGKKAVEFDFNKYMDSKAMTVNEALNKAIPLRYP
QKIYESMRYSLLAGGKRVRPVLCIAACELVGGTEELAIPTACAIEMIHTMSLM
HDDLPCIDNDDLRRGKPTNHKIFGEDTAVTAGNALHSYAFEHIAVSTSKTVG
ADRILRMVSELGRATGSEGVMGGQMVDIASEGDPSIDLQTLEWIHIHKTAML
LECSVVCGAIIGGASEIVIERARRYARCVGLLFQVVDDILDVTKSSDELGKTAG
KDLISDKATYPKLMGLEKAKEFSDELLNRAKGELSCFDPVKAAPLLGLADYV
AFRQN
>CgGPPS2 (SEQ ID NO: 37)
MKDVSLSSFDAHDLDLDKFPEVVRDRLTQFLDAQELTIADIGAP
VTDAVAHLRSFVLNGGKRIRPLYAWAGFLAAQGHKNSSEKLESVLDAAASL
EFIQACALIHDDIIDSSDTRRGAPTVHRAVEADHRANNFEGDPEHFGVSVSILA
GDMALVWAEDMLQDSGLSAEALARTRDAWRGMRTEVIGGQLLDIYLESHA
NESVELADSVNRFKTAAYTIARPLHLGASIAGGSPQLIDALLHYGHDIGIAFQL
RDDLLGVFGDPAITGKPAGDDIREGKRTVLLALALQRADKQSPEAATAIRAG
VGKVTSPEDIAVITEHIRATGAEEEVEQRISQLTESGLAHLDDVDIPDEVRAQL
RALAIRSTERRM
>PfLS from Perilla frutescens (SEQ ID NO: 38)
MHMAIPIKPAHYLHNSGRSYASQLCGFSSTSTRAAIARLPLCLR
FRCSLQASDQRRSGNYSPSFWNADYILSLNSHYKDKSHMKRAGELIVQVKM
VMGKETDPVVQLELIDDLQKLALSHHVEKEIKEILFKISTYDHKIMVERDLYS
TALAFRLLRQYGFKVPQEVFDCFKNDNGEFKRSLSSDTKGLLQLYEASFLLTE
GEMTLELAREFATKSLQEKLNEKTIDDDDDADTNLISCVRHSLDIPIHWRIQRP
NASWWIDAYKRRSHMNPLVLELAKLDLNIFQAQFQQELKQDLGWWKNTCL
AEKLPFVRDRLVECYFWCTGIIQPLQHENARVTLAKVNALITTLDDIYDVYGT
LEELELFTEAIRRWDVSSIDHLPNYMQLCFLALNNFVDDTAYDVMKEKDINII
PYLRKSWLDLAETYLVEAKWFYSGHKPNLEEYLNNAWISISGPVMLCHVFFR
VTDSITRETVESLFKYHDLIRYSSTILRLADDLGTSLEEVSRGDVPKSIQCYMN
DNNASEEEARRHIRWLIAETWKKINEEVWSVDSPFCKDFIACAADMGRMAQF
MYHNGDGHGIQNPQIHQQMTDILFEQWL
>QiMyrS from Quercus ilex (SEQ ID NO: 39)
MMVANKVSTSPDILRRSANYQPSIWNHDYIESLRIEYVGETCTR
QINVLKEQVRMMLHKVVNPLEQLELIEILQRLGLSYHFEEEIKRILDGVYNND
HGGDTWKAENLYATALKFRLLRQHGYSVSQEVFNSFKDERGSFKACLCEDT
KGMLSLYEASFFLIEGENILEEARDFSTKHLEEYVKQNKEKNLATLVNHSLEF
PLHWRMPRLEARWFINIYRHNQDVNPILLEFAELDFNIVQAAHQADLKQVST
WWKSTGLVENLSFARDRPVENFFWTVGLIFQPQFGYCRRMFTKVFALITTIDD
VYDVYGTLDELELFTDVVERWDINAMDQLPDYMKICFLTLHNSVNEMALDT
MKEQRFHIIKYLKKAWVDLCRYYLVEAKWYSNKYRPSLQEYIENAWISIGAP
TILVHAYFFVTNPITKEALDCLEEYPNIIRWSSIIARLADDLGTSTDELKRGDVP
KAIQCYMNETGASEEGAREYIKYLISATWKKMNKDRAASSPFSHIFIEIALNLA
RMAQCLYQHGDGHGLGNRETKDRILSLLIQPIPLNKD
>SfCinS1 from Salvia fruticosa (SEQ ID NO: 40).
MSLQTGNEIQTERRTGGYQPTLWDFSTIQSFDSEYKEEKHLMR
AAGMIDQVKMMLQEEVDSIRRLELIDDLRRLGISCHFEREIVEILNSKYYTNNE
IDERDLYSTALRFRLLRQYDFSVSQEVFDCFKNAKGTDFKPSLVDDTRGLLQL
YEASFLSAQGEETLRLARDFATKFLQKRVLVDKDINLLSSIERALELPTHWRV
QMPNARSFIDAYKRRPDMNPTVLELAKLDENMVQAQFQQELKEASRWWNS
TGLVHELPFVRDRIVECYYWTTGVVERRQHGYERIMLTKINALVTTIDDVFDI
YGTLEELQLFTTAIQRWDIESMKQLPPYMQICYLALFNFVNEMAYDTLRDKG
FDSTPYLRKVWVGLIESYLIEAKWYYKGHKPSLEEYMKNSWISIGGIPILSHLF
FRLTDSIEEEAAESMHKYHDIVRASCTILRLADDMGTSLDEVERGDVPKSVQC
YMNEKNASEEEAREHVRSLIDQTWKMMNKEMMTSSFSKYFVEVSANLARM
AQWIYQHESDGFGMQHSLVNKMLRDLLFHRYE
>ACS1 (SEQ ID NO: 41).
MSEDHPAIHPPSEFKDNHPHFGGPHLDCLQDYHQLHKESIEDPK
AFWKKMANELISWSTPFETVRSGGFEHGDVAWFPEGQLNASYNCVDRHAFA
NPDKPAIIFEADEPGQGRIVTYGELLRQVSQVAATLRSFGVQKGDTVAVYLP
MIPEAIVTLLAITRIGAVHSVIFAGFSSGSLRDRINDAKSKVVVTTDASMRGGK
TIDTKKIVDEALRDCPSVTHTLVFRRAGVENLAWTEGRDFWWHEEVVKHRP
YLAPVPVASEDPIFLLYTSGSTGTPKGLAHATGGYLLGAALTAKYVFDIHGDD
KLFTAGDVGWITGHTYVLYGPLMLGATTVVFEGTPAYPSFSRYWDIVDDHKI
THEYVAPTALRLLKRAGTHHIKHDLSSLRTLGSVGEPIAPDVWQWYNDNIGR
GKAHICDTYWQTETGSHIIAPMAGVTPTKPGSASLPVFGIDPVIIDPVSGEELK
GNNVEGVLALRSPWPSMARTVWNTHERYMETYLRPYPGYYFTGDGAARDN
DGFYWIRGRVDDVVNVSGHRLSTAEIEAALIEHAQVSESAVVGVHDDLTGQA
VNAFVALKNPVEDVDALRKELVVQVRKTIGPFAAPKNVIIVDDLPKTRSGKI
MRRILRKVLAGEEDQLGDISTLANPDVVQTIIEVVHSLKK
>PTA (SEQ ID NO: 42)
MSIIQNIEKAKSDKKKIVLPEGAEPRTLKAAEIVLKEGIADLVLL
GNEDEIRNAAKDLDISKAEIIDPVKSEMFDRYANDFYELRKNKGITLEKARETI
KDNIYFGCMMVKEGYADGLVSGAIHATADLLRPAFQIIKTAPGAKIVSSFFIM
EVPNCEYGENGVFLFADCAVNPSPNAEELASIAVQSANTAKNLLGFEPKVAM
LSFSTKGSASHELVDKVRKATEIAKELMPDVAIDGELQLDAALVKEVAELKA
PGSKVAGCANVLIFPDLQAGNIGYKLVQRLAKANAIGPITQGMGAPVNDLSR
GCSYRDIVDVIATTAVQAQ
>XPK (SEQ ID NO: 43)
MQSIIGKHKDEGKITPEYLKKIDAYWRAANFISVGQLYLLDNPL
LREPLKPEHLKRKVVGHWGTIPGQNFIYAHLNRVIKKYDLDMIYVSGPGHGG
QVMVSNSYLDGTYSEVYPNVSRDLNGLKKLCKQFSFPGGISSHMAPETPGSIN
EGGELGYSLAHSFGAVFDNPDLITACVVGDGEAETGPLATSWQANKFLNPVT
DGAVLPILHLNGYKISNPTVLSRIPKDELEKFFEGNGWKPYFVEGEDPETMHK
LMAETLDIVTEEILNIQKNARENNDCSRPKWPMIVLRTPKGWTGPKFVDGVP
NEGSFRAHQVPLAVDRYHTENLDQLEEWLKSYKPEELFDENYRLIPELEELTP
KGNKRMAANLHANGGLLLRELRTPDFRDYAVDVPTPGSTVKQDMIELGKYV
RDVVKLNEDTRNFRIFGPDETMSNRLWAVFEGTKRQWLSEIKEPNDEFLSND
GRIVDSMLSEHLCEGWLEGYLLTGRHGFFASYEAFLRIVDSMITQHGKWLKV
TSQLPWRKDIASLNLIATSNVWQQDHNGYTHQDPGLLGHIVDKKPEIVRAYL
PADANTLLAVFDKCLHTKHKINLLVTSKHPRQQWLTMDQAVKHVEQGISIW
DWASNDKGQEPDVVIASCGDTPTLEALAAVTILHEHLPELKVRFVNVVDMM
KLLPENEHPHGLSDKDYNALFTTDKPVIFAFHGFAHLINQLTYHRENRNLHVH
GYMEEGTITTPFDMRVQNKLDRFNLVKDVVENLPQLGNRGAHLVQLMNDK
LVEHNQYIREVGEDLPEITNWQWHV
>ACC1 (SEQ ID NO: 44)
MRLQLRTLTRRFFSMASGSSTPDVAPLVDPNIHKGLASHFFGLN
SVHTAKPSKVKEFVASHGGHTVINKVLIANNGIAAVKEIRSVRKWAYETFGD
ERAISFTVMATPEDLAANADYIRMADQYVEVPGGTNNNNYANVELIVDVAE
RSGVDAVWAGWGHASENPLLPESLAASPRKIVFIGPPGAAMRSLGDKISSTIV
AQHAKVPCIPWSGTGVDEVVVDKSTNLVSVSEEVYTKGCTTGPKQGLEKAK
QIGFPVMIKASEGGGGKGIRKVEREEDFEAAYHQVEGEIPGSPIFIMQLAGNAR
HLEVQLLADQYGNNISLFGRDCSVQRRHQKIIEEAPVTVAGQQTFTAMEKAA
VRLGKLVGYVSAGTVEYLYSHEDDKFYFLELNPRLQVEHPTTEMVTGVNLP
AAQLQIAMGIPLDRIKDIRLFYGVNPHTTTPIDFDFSGEDADKTQRRPVPRGHT
TACRITSEDPGEGFKPSGGTMHELNFRSSSNVWGYFSVGNQGGIHSFSDSQFG
HIFAFGENRSASRKHMVVALKELSIRGDFRTTVEYLIKLLETPDFEDNTITTGW
LDELISNKLTAERPDSFLAVVCGAATKAHRASEDSIATYMASLEKGQVPARDI
LKTLFPVDFIYEGQRYKFTATRSSEDSYTLFINGSRCDIGVRPLSDGGILCLVG
GRSHNVYWKEEVGATRLSVDSKTCLLEVENDPTQLRSPSPGKLVKFLVENGD
HVRANQPYAEIEVMKMYMTLTAQEDGIVQLMKQPGSTIEAGDILGILALDDP
SKVKHAKPFEGQLPELGPPTLSGNKPHQRYEHCQNVLHNILLGFDNQVVMKS
TLQEMVGLLRNPELPYLQWAHQVSSLHTRMSAKLDATLAGLIDKAKQRGGE
FPAKQLLRALEKEASSGEVDALFQQTLAPLFDLAREYQDGLAIHELQVAAGL
LQAYYDSEARFCGPNVRDEDVILKLREENRDSLRKVVMAQLSHSRVGAKNN
LVLALLDEYKVADQAGTDSPASNVHVAKYLRPVLRKIVELESRASAKVSLKA
REILIQCALPSLKERTDQLEHILRSSVVESRYGEVGLEHRTPRADILKEVVDSK
YIVFDVLAQFFAHDDPWIVLAALELYIRRACKAYSILDINYHQDSDLPPVISWR
FRLPTMSSALYNSVVSSGSKTPTSPSVSRADSVSDFSYTVERDSAPARTGAIVA
VPHLDDLEDALTRVLENLPKRGAGLAISVGASNKSAAASARDAAAAAASSV
DTGLSNICNVMIGRVDESDDDDTLIARISQVIEDFKEDFEACSLRRITESFGNSR
GTYPKYFTFRGPAYEEDPTIRHIEPALAFQLELARLSNFDIKPVHTDNRNIHVY
EATGKNAASDKRFFTRGIVRPGRLRENIPTSEYLISEADRLMSDILDALEVIGTT
NSDLNHIFINFSAVFALKPEEVEAAFGGFLERFGRRLWRLRVTGAEIRMMVSD
PETGSAFPLRAMINNVSGYVVQSELYAEAKNDKGQWIFKSLGKPGSMHMRSI
NTPYPTKEWLQPKRYKAHLMGTTYCYDFPELFRQSIESDWKKYDGKAPDDL
MTCNELILDEDSGELQEVNREPGANNVGMVAWKFEAKTPEYPRGRSFIVVAN
DITFQIGSFGPAEDQFFFKVTELARKLGIPRIYLSANSGARIGIADELVGKYKVA
WNDETDPSKGFKYLYFTPESLATLKPDTVVTTEIEEEGPNGVEKRHVIDYIVG
EKDGLGVECLRGSGLIAGATSRAYKDIFTLTLVTCRSVGIGAYLVRLGQRAIQI
EGQPIILTGAPAINKLLGREVYSSNLQLGGTQIMYNNGVSHLTARDDLNGVHK
IMQWLSYIPASRGLPVPVLPHKTDVWDRDVTFQPVRGEQYDVRWLISGRTLE
DGAFESGLFDKDSFQETLSGWAKGVVVGRARLGGIPFGVIGVETATVDNTTP
ADPANPDSIEMSTSEAGQVWYPNSAFKTSQAINDFNHGEALPLMILANWRGF
SGGQRDMYNEVLKYGSFIVDALVDYKQPIMVYIPPTGELRGGSWVVVDPTIN
SDMMEMYADVESRGGVLEPEGMVGIKYRRDKLLDTMARLDPEYSSLKKQLE
ESPDSEELKVKLSVREKSLMPIYQQISVQFADLHDRAGRMEAKGVIREALVW
KDARRFFFWRIRRRLVEEYLITKINSILPSCTRLECLARIKSWKPATLDQGSDR
GVAEWFDENSDAVSARLSELKKDASAQSFASQLRKDRQGTLQGMKQALASL
SEAERAELLKGL
>ACC1.1 (SEQ ID NO: 45)
MRLQLRTLTRRFFSMASGSSTPDVAPLVDPNIHKGLASHFFGLN
SVHTAKPSKVKEFVASHGGHTVINKVLIANNGIAAVKEIRSVRKWAYETFGD
ERAISFTVMATPEDLAANADYIRMADQYVEVPGGTNNNNYANVELIVDVAE
RSGVDAVWAGWGHASENPLLPESLAASPRKIVFIGPPGAAMRSLGDKISSTIV
AQHAKVPCIPWSGTGVDEVVVDKSTNLVSVSEEVYTKGCTTGPKQGLEKAK
QIGFPVMIKASEGGGGKGIRKVEREEDFEAAYHQVEGEIPGSPIFIMQLAGNAR
HLEVQLLADQYGNNISLFGRDCSVQRRHQKIIEEAPVTVAGQQTFTAMEKAA
VRLGKLVGYVSAGTVEYLYSHEDDKFYFLELNPRLQVEHPTTEMVTGVNLP
AAQLQIAMGIPLDRIKDIRLFYGVNPHTTTPIDFDFSGEDADKTQRRPVPRGHT
TACRITSEDPGEGFKPSGGTMHELNFRSSSNVWGYFSVGNQGGIHSFSDSQFG
HIFAFGENRSASRKHMVVALKELSIRGDFRTTVEYLIKLLETPDFEDNTITTGW
LDELISNKLTAERPDSFLAVVCGAATKAHRASEDSIATYMASLEKGQVPARDI
LKTLFPVDFIYEGQRYKFTATRSSEDSYTLFINGSRCDIGVRPLRDGGILCLVG
GRSHNVYWKEEVGATRLRVDSKTCLLEVENDPTQLRSPSPGKLVKFLVENGD
HVRANQPYAEIEVMKMYMTLTAQEDGIVQLMKQPGSTIEAGDILGILALDDP
SKVKHAKPFEGQLPELGPPTLSGNKPHQRYEHCQNVLHNILLGFDNQVVMKS
TLQEMVGLLRNPELPYLQWAHQVSSLHTRMSAKLDATLAGLIDKAKQRGGE
FPAKQLLRALEKEASSGEVDALFQQTLAPLFDLAREYQDGLAIHELQVAAGL
LQAYYDSEARFCGPNVRDEDVILKLREENRDSLRKVVMAQLSHSRVGAKNN
LVLALLDEYKVADQAGTDSPASNVHVAKYLRPVLRKIVELESRASAKVSLKA
REILIQCALPSLKERTDQLEHILRSSVVESRYGEVGLEHRTPRADILKEVVDSK
YIVFDVLAQFFAHDDPWIVLAALELYIRRACKAYSILDINYHQDSDLPPVISWR
FRLPTMSSALYNSVVSRGSKTPTSPSVSRADSVSDFSYTVERDSAPARTGAIVA
VPHLDDLEDALTRVLENLPKRGAGLAISVGASNKSAAASARDAAAAAASSV
DTGLSNICNVMIGRVDESDDDDTLIARISQVIEDFKEDFEACSLRRITFSFGNSR
GTYPKYFTFRGPAYEEDPTIRHIEPALAFQLELARLSNFDIKPVHTDNRNIHVY
EATGKNAASDKRFFTRGIVRPGRLRENIPTSEYLISEADRLMSDILDALEVIGTT
NSDLNHIFINFSAVFALKPEEVEAAFGGFLERFGRRLWRLRVTGAEIRMMVSD
PETGSAFPLRAMINNVSGYVVQSELYAEAKNDKGQWIFKSLGKPGSMHMRSI
NTPYPTKEWLQPKRYKAHLMGTTYCYDFPELFRQSIESDWKKYDGKAPDDL
MTCNELILDEDSGELQEVNREPGANNVGMVAWKFEAKTPEYPRGRSFIVVAN
DITFQIGSFGPAEDQFFFKVTELARKLGIPRIYLSANSGARIGIADELVGKYKVA
WNDETDPSKGFKYLYFTPESLATLKPDTVVTTEIEEEGPNGVEKRHVIDYIVG
EKDGLGVECLRGSGLIAGATSRAYKDIFTLTLVTCRSVGIGAYLVRLGQRAIQI
EGQPIILTGAPAINKLLGREVYSSNLQLGGTQIMYNNGVSHLTARDDLNGVHK
IMQWLSYIPASRGLPVPVLPHKTDVWDRDVTFQPVRGEQYDVRWLISGRTLE
DGAFESGLFDKDSFQETLSGWAKGVVVGRARLGGIPFGVIGVETATVDNTTP
ADPANPDSIEMSTSEAGQVWYPNSAFKTSQAINDFNHGEALPLMILANWRGF
SGGQRDMYNEVLKYGSFIVDALVDYKQPIMVYIPPTGELRGGSWVVVDPTIN
SDMMEMYADVESRGGVLEPEGMVGIKYRRDKLLDTMARLDPEYSSLKKQLE
ESPDSEELKVKLSVREKSLMPIYQQISVQFADLHDRAGRMEAKGVIREALVW
KDARRFFFWRIRRRLVEEYLITKINSILPSCTRLECLARIKSWKPATLDQGSDR
GVAEWFDENSDAVSARLSELKKDASAQSFASQLRKDRQGTLQGMKQALASL
SEAERAELLKGL*
>mvaE (SEQ ID NO: 46)
MKTVVIIDALRTPIGKYKGSLSQVSAVDLGTHVTTQLLKRHSTI
SEEIDQVIFGNVLQAGNGQNPARQIAINSGLSHEIPAMTVNEVCGSGMKAVIL
AKQLIQLGEAEVLIAGGIENMSQAPKLQRFNYETESYDAPFSSMMYDGLTDA
FSGQAMGLTAENVAEKYHVTREEQDQFSVHSQLKAAQAQAEGIFADEIAPLE
VSGTLVEKDEGIRPNSSVEKLGTLKTVFKEDGTVTAGNASTINDGASALIIASQ
EYAEAHGLPYLAIIRDSVEVGIDPAYMGISPIKAIQKLLARNQLTTEEIDLYEIN
EAFAATSIVVQRELALPEEKVNIYGGGISLGHAIGATGARLLTSLSYQLNQKE
KKYGVASLCIGGGLGLAMLLERPQQKKNSRFYQMSPEERLASLLNEGQISAD
TKKEFENTALSSQIANHMIENQISETEVPMGVGLHLTVDETDYLVPMATEEPS
VIAALSNGAKIAQGFKTVNQQRLMRGQIVFYDVADAESLIDELQVRETEIFQQ
AELSYPSIVKRGGGLRDLQYRAFDESFVSVDFLVDVKDAMGANIVNAMLEG
VAELFREWFAEQKILFSILSNYATESVVTMKTAIPVSRLSKGSNGREIAEKIVL
ASRYASLDPYRAVTHNKGIMNGIEAVVLATGNDTRAVSASCHAFAVKEGRY
QGLTSWTLDGEQLIGEISVPLALATVGGATKVLPKSQAAADLLAVTDAKELS
RVVAAVGLAQNLAALRALVSEGIQKGHMALQARSLAMTVGATGKEVEAVA
QQLKRQKTMNQDRALAILNDLRKQ*
MPT4.1 (SEQ ID NO: 47)
MSDNSIATKILNFGHTCWKLQRPYAVKGMISIACGLFGRELFNN
RHLFSWGLMWKAFFALVPILSFNFFAAIMNQIYDVDIDRINKPDLPLVSGEMSI
ETAWILSIIVALTGLIVTIKLKSAPLFVFIYIFGIFAGFAYSVPPIRWKQYPFTNFL
ITISSHVGLAFTSYSATTSALGLPFVWRPAFSFIIAFMTVMGMTIAFAKDISDIE
GDAKYGVSTVATKLGARNMTFVVSGVLLLNYLVSISIGIIWPQVFKSNIMILS
HAILAFCLIFQTRELALANYASAPSRQFFEFIWLLYYAEYFVYVFI
MPT21.9 (SEQ ID NO: 48)
MSDNSIATKILNFGHTCWKLQRPYVVKGMISIACGLFGKELLH
NTNLISWGLMWKAFFALVPILSFNFFASIMNQIYDVDIDRINKPDLPLVSGEMS
IETAWILSIIVALTGLIVTIKLKSAPLFVFIYIFGIFAGFAYSVPPIRWKQNPSTNF
LITISSHVGLAFTSYYASRAALGLPFELRPSFTFLLAFMTVMGMTIAFAKDISDI
EGDAKYGVSTVATKLGARNMTFVVSGVLLLNYVAAILAGIIWPQAFNSNVM
LLSHAILAFCLIFQTRELALANYASAPSRQFFEFIWLLYYAEYFVYVFI
APT 73.81 (SEQ ID NO: 49)
MDEVYAAVEQTSRLLDVPCSPDRFEPVWKAFGDQLPDSHLVFS
MAAGEAHRGELDFDFSLRPEGADPYTTALEHGFIEPTDHPVGSVLAEVGKRF
AIASYGVEYGVVGGFKKSYAFFPLDDFPPLAQFAEVPSVPPCLAGHVETLTRL
GFDDKVSIIGVNYRKNTLNVYLAASAVDTGDKLALLRAFGYPEPDARVRQFI
ERSFRLYPTFNWDSSAAERICFAVHTQQPGELPAPHDEPTEAFARQVPHVYEG
GREFVSGVALAPSGASYYKLAALYQKGRRCLD
HMG1 (SEQ ID NO: 50)
MLQAAIGKIVGFAVNRPIHTVVLTSIVASTAYLAILDIAIPGFEGT
QPISYYHPAAKSYDNPADWTHIAEADIPSDAYRLAFAQIRVSDVQGGEAPTIP
GAVAVSDLDHRIVMDYKQWAPWTASNEQIASENHIWKHSFKDHVAFSWIK
WFRWAYLRLSTLIQGADNFDIAVVALGYLAMHYTFFSLFRSMRKVGSHFWL
ASMALVSSTFAFLLAVVASSSLGYRPSMITMSEGLPFLVVAIGFDRKVNLASE
VLTSKSSQLAPMVQVITKIASKALFEYSLEVAALFAGAYTGVPRLSQFCFLSA
WILIFDYMFLLTFYSAVLAIKFEINHIKRNRMIQDALKEDGVSAAVAEKVADS
SPDAKLDRKSDVSLFGASGAIAVFKIFMVLGFLGLNLINLTAIPHLGKAAAAA
QSVTPITLSPELLHAIPASVPVVVTFVPSVVYEHSQLILQLEDALTTFLAACSKT
IGDPVISKYIFLCLMVSTALNVYLFGATREVVRTQSVKVVEKHVPIVIEKPSEK
EEDTSSEDSIELTVGKQPKPVTETRSLDDLEAIMKAGKTKLLEDHEVVKLSLE
GKLPLYALEKQLGDNTRAVGIRRSIISQQSNTKTLETSKLPYLHYDYDRVFGA
CCENVIGYMPLPVGVAGPMNIDGKNYHIPMATTEGCLVASTMRGCKAINAG
GGVTTVLTQDGMTRGPCVSFPSLKRAGAAKIWLDSEEGLKSMRKAFNSTSRF
ARLQSLHSTLAGNLLFIRFRTTTGDAMGMNMISKGVEHSLAVMVKEYGFPD
MDIVSVSGNYCTDKKPAAINWIEGRGKSVVAEATIPAHIVKSVLKSEVDALVE
LNISKNLIGSAMAGSVGGFNAHAANLVTAIYLATGQDPAQNVESSNCITLMS
NVDGNLLISVSMPSIEVGTIGGGTILEPQGAMLEMLGVRGPHIETPGANAQQL
ARIIASGVLAAELSLCSALAAGHLVQSHMTHNRSQAPTPAKQSQADLQRLQN
GSNICIRS
tHMG1 (SEQ ID NO: 51)
MREVVRTQSVKVVEKHVPIVIEKPSEKEEDTSSEDSIELTVGKQ
PKPVTETRSLDDLEAIMKAGKTKLLEDHEVVKLSLEGKLPLYALEKQLGDNT
RAVGIRRSIISQQSNTKTLETSKLPYLHYDYDRVFGACCENVIGYMPLPVGVA
GPMNIDGKNYHIPMATTEGCLVASTMRGCKAINAGGGVTTVLTQDGMTRGP
CVSFPSLKRAGAAKIWLDSEEGLKSMRKAFNSTSRFARLQSLHSTLAGNLLFI
RFRTTTGDAMGMNMISKGVEHSLAVMVKEYGFPDMDIVSVSGNYCTDKKPA
AINWIEGRGKSVVAEATIPAHIVKSVLKSEVDALVELNISKNLIGSAMAGSVG
GFNAHAANLVTAIYLATGQDPAQNVESSNCITLMSNVDGNLLISVSMPSIEVG
TIGGGTILEPQGAMLEMLGVRGPHIETPGANAQQLARIIASGVLAAELSLCSAL
AAGHLVQSHMTHNRSQAPTPAKQSQADLQRLQNGSNICIRS
IDI1 (SEQ ID NO: 52)
MTTSYSDKIKSISASSVAQQFPEVAPIADVSKASRPSTESSDSSA
KLFDGHDEEQIKLMDEICVVLDWDDKPIGGASKKCCHLMDNINDGLVHRAFS
VFMFNDRGELLLQQRAAEKITFANMWTNTCCSHPLAVPSEMGGLDLESRIQG
AKNAAVRKLEHELGIDPKAVPADKFHFLTRIHYAAPSSGPWGEHEIDYILFVR
GDPELKVVANEVRDTVWVSQQGLKDMMADPKLVFTPWFRLICEQALFPWW
DQLDNLPAGDDEIRRWIK
MPT21.1 (SEQ ID NO: 53)
MSDNSIATKILNFGHTCWKLQRPFVVKGMISIACGLFGKELLHNTNLI
SWGLMWKAFFALVPILSFNFFAAIMNQIYDVDIDRINKPDLPLVSGEMSIETAWILSII
VALTGLIVTIKLKSAPLFVFIYIFGIFAGFAYSVPPIRWKQNPSTNFLITISSHVGLAFTSYY
ASRAALGLPFELRPSFTFLLAFMTVMGMTIAFAKDISDIEGDAKYGVSTVATKLG
ARNMTFVVSGVLLLNYVAAILAGIIWPQAFNSNVMLLSHAILAFCLIFQTRELALANY
ASAPSRQFFEFIWLLYYAEYFVYVFI
MPT21.2 (SEQ ID NO: 54)
MSDNSIATKILNFGHTCWKLQRPMVVKGMISIACGLFGKELLHNTNLI
SWGLMWKAFFALVPILSFNFFAAIMNQIYDVDIDRINKPDLPLVSGEMSIETAWILSII
VALTGLIVTIKLKSAPLFVFIYIFGIFAGFAYSVPPIRWKQNPSTNFLITISSHVGLAFTSYY
ASRAALGLPFELRPSFTFLLAFMTVMGMTIAFAKDISDIEGDAKYGVSTVATKLG
ARNMTFVVSGVLLLNYVAAILAGIIWPQAFNSNVMLLSHAILAFCLIFQTRELALANY
ASAPSRQFFEFIWLLYYAEYFVYVFI
MPT21.3 (SEQ ID NO: 55)
MSDNSIATKILNFGHTCWKLQRPYAVKGMISIACGLFGKELLHNTNLI
SWGLMWKAFFALVPILSFNFFAAIMNQIYDVDIDRINKPDLPLVSGEMSIETAWILSII
VALTGLIVTIKLKSAPLFVFIYIFGIFAGFAYSVPPIRWKQNPSTNFLITISSHVGLAFTSYY
ASRAALGLPFELRPSFTFLLAFMTVMGMTIAFAKDISDIEGDAKYGVSTVATKLG
ARNMTFVVSGVLLLNYVAAILAGIIWPQAFNSNVMLLSHAILAFCLIFQTRELALANY
ASAPSRQFFEFIWLLYYAEYFVYVFI
MPT21.4 (SEQ ID NO: 56)
MSDNSIATKILNFGHTCWKLQRPYVVKGAISIACGLFGKELLHNTNLI
SWGLMWKAFFALVPILSFNFFAAIMNQIYDVDIDRINKPDLPLVSGEMSIETAWILSII
VALTGLIVTIKLKSAPLFVFIYIFGIFAGFAYSVPPIRWKQNPSTNFLITISSHVGLAFTS
YYASRAALGLPFELRPSFTFLLAFMTVMGMTIAFAKDISDIEGDAKYGVSTVATKLG
ARNMTFVVSGVLLLNYVAAILAGIIWPQAFNSNVMLLSHAILAFCLIFQTRELALANY
ASAPSRQFFEFIWLLYYAEYFVYVFI
MPT21.5 (SEQ ID NO: 57)
MSDNSIATKILNFGHTCWKLQRPYVVKGMITIACGLFGKELLHNTNLI
SWGLMWKAFFALVPILSFNFFAAIMNQIYDVDIDRINKPDLPLVSGEMSIETAWILSII
VALTGLIVTIKLKSAPLFVFIYIFGIFAGFAYSVPPIRWKQNPSTNFLITISSHVGLAFTS
YYASRAALGLPFELRPSFTFLLAFMTVMGMTIAFAKDISDIEGDAKYGVSTVATKLG
ARNMTFVVSGVLLLNYVAAILAGIIWPQAFNSNVMLLSHAILAFCLIFQTRELALANY
ASAPSRQFFEFIWLLYYAEYFVYVFI
MPT21.6 (SEQ ID NO: 58)
MSDNSIATKILNFGHTCWKLQRPYVVKGMIVIACGLFGKELLHNTNLI
SWGLMWKAFFALVPILSFNFFAAIMNQIYDVDIDRINKPDLPLVSGEMSIETAWILSII
VALTGLIVTIKLKSAPLFVFIYIFGIFAGFAYSVPPIRWKQNPSTNFLITISSHVGLAFTS
YYASRAALGLPFELRPSFTFLLAFMTVMGMTIAFAKDISDIEGDAKYGVSTVATKLG
ARNMTFVVSGVLLLNYVAAILAGIIWPQAFNSNVMLLSHAILAFCLIFQTRELALANY
ASAPSRQFFEFIWLLYYAEYFVYVFI
MPT21.7 (SEQ ID NO: 59)
MSDNSIATKILNFGHTCWKLQRPYVVKGMIAIACGLFGKELLHNTNLI
SWGLMWKAFFALVPILSFNFFAAIMNQIYDVDIDRINKPDLPLVSGEMSIETAWILSII
VALTGLIVTIKLKSAPLFVFIYIFGIFAGFAYSVPPIRWKQNPSTNFLITISSHVGLAFTS
YYASRAALGLPFELRPSFTFLLAFMTVMGMTIAFAKDISDIEGDAKYGVSTVATKLG
ARNMTFVVSGVLLLNYVAAILAGIIWPQAFNSNVMLLSHAILAFCLIFQTRELALANY
ASAPSRQFFEFIWLLYYAEYFVYVFI
MPT21.8 (SEQ ID NO: 60)
MSDNSIATKILNFGHTCWKLQRPYVVKGMISIACGLFGKELLHNTNLI
SWGLMWKAFFALVPILSFNFFAGIMNQIYDVDIDRINKPDLPLVSGEMSIETAWILSII
VALTGLIVTIKLKSAPLFVFIYIFGIFAGFAYSVPPIRWKQNPSTNFLITISSHVGLAFTS
YYASRAALGLPFELRPSFTFLLAFMTVMGMTIAFAKDISDIEGDAKYGVSTVATKLG
ARNMTFVVSGVLLLNYVAAILAGIIWPQAFNSNVMLLSHAILAFCLIFQTRELALANY
ASAPSRQFFEFIWLLYYAEYFVYVFI
MPT21.10 (SEQ ID NO: 61)
MSDNSIATKILNFGHTCWKLQRPYVVKGMISIACGLFGKELLHNTNLI
SWGLMWKAFFALVPILSFNFFAAIMNQIYDVDMDRINKPDLPLVSGEMSIETAWILSII
VALTGLIVTIKLKSAPLFVFIYIFGIFAGFAYSVPPIRWKQNPSTNFLITISSHVGLAFTS
YYASRAALGLPFELRPSFTFLLAFMTVMGMTIAFAKDISDIEGDAKYGVSTVATKLG
ARNMTFVVSGVLLLNYVAAILAGIIWPQAFNSNVMLLSHAILAFCLIFQTRELALANY
ASAPSRQFFEFIWLLYYAEYFVYVFI
MPT21.11 (SEQ ID NO: 62)
MSDNSIATKILNFGHTCWKLQRPYVVKGMISIACGLFGKELLHNTNLI
SWGLMWKAFFALVPILSFNFFAAIMNQIYDVDIDRVNKPDLPLVSGEMSIETAWILSII
VALTGLIVTIKLKSAPLFVFIYIFGIFAGFAYSVPPIRWKQNPSTNFLITISSHVGLAFTS
YYASRAALGLPFELRPSFTFLLAFMTVMGMTIAFAKDISDIEGDAKYGVSTVATKLG
ARNMTFVVSGVLLLNYVAAILAGIIWPQAFNSNVMLLSHAILAFCLIFQTRELALANY
ASAPSRQFFEFIWLLYYAEYFVYVFI
MPT21.12 (SEQ ID NO: 63)
MSDNSIATKILNFGHTCWKLQRPYVVKGMISIACGLFGKELLHNTNLI
SWGLMWKAFFALVPILSFNFFAAIMNQIYDVDIDRINKPDLPLVSGEMSIETAWILSII
VALTGLIVTIKLKSAPLFVFIYIFGIFAGFAYSVPPIRWKQNPSTNFEITISSHVGLAFTS
YYASRAALGLPFELRPSFTFLLAFMTVMGMTIAFAKDISDIEGDAKYGVSTVATKLG
ARNMTFVVSGVLLLNYVAAILAGIIWPQAFNSNVMLLSHAILAFCLIFQTRELALANY
ASAPSRQFFEFIWLLYYAEYFVYVFI
MPT21.13 (SEQ ID NO: 64)
MSDNSIATKILNFGHTCWKLQRPYVVKGMISIACGLFGKELLHNTNLI
SWGLMWKAFFALVPILSFNFFAAIMNQIYDVDIDRINKPDLPLVSGEMSIETAWILSII
VALTGLIVTIKLKSAPLFVFIYIFGIFAGFAYSVPPIRWKQNPSTNFLITIASHVGLAFTS
YYASRAALGLPFELRPSFTFLLAFMTVMGMTIAFAKDISDIEGDAKYGVSTVATKLG
ARNMTFVVSGVLLLNYVAAILAGIIWPQAFNSNVMLLSHAILAFCLIFQTRELALANY
ASAPSRQFFEFIWLLYYAEYFVYVFI
MPT21.14 (SEQ ID NO: 65)
MSDNSIATKILNFGHTCWKLQRPYVVKGMISIACGLFGKELLHNTNLI
SWGLMWKAFFALVPILSFNFFAAIMNQIYDVDIDRINKPDLPLVSGEMSIETAWILSII
VALTGLIVTIKLKSAPLFVFIYIFGIFAGFAYSVPPIRWKQNPSTNFLITIGSHVGLAFTS
YYASRAALGLPFELRPSFTFLLAFMTVMGMTIAFAKDISDIEGDAKYGVSTVATKLG
ARNMTFVVSGVLLLNYVAAILAGIIWPQAFNSNVMLLSHAILAFCLIFQTRELALANY
ASAPSRQFFEFIWLLYYAEYFVYVFI
MPT21.15 (SEQ ID NO: 66)
MSDNSIATKILNFGHTCWKLQRPYVVKGMISIACGLFGKELLHNTNLI
SWGLMWKAFFALVPILSFNFFAAIMNQIYDVDIDRINKPDLPLVSGEMSIETAWILSII
VALTGLIVTIKLKSAPLFVFIYIFGIFAGFAYSVPPIRWKQNPSTNFLITIVSHVGLAFTS
YYASRAALGLPFELRPSFTFLLAFMTVMGMTIAFAKDISDIEGDAKYGVSTVATKLG
ARNMTFVVSGVLLLNYVAAILAGIIWPQAFNSNVMLLSHAILAFCLIFQTRELALANY
ASAPSRQFFEFIWLLYYAEYFVYVFI
MPT21.16 (SEQ ID NO: 67)
MSDNSIATKILNFGHTCWKLQRPYVVKGMISIACGLFGKELLHNTNLI
SWGLMWKAFFALVPILSFNFFAAIMNQIYDVDIDRINKPDLPLVSGEMSIETAWILSII
VALTGLIVTIKLKSAPLFVFIYIFGIFAGFAYSVPPIRWKQNPSTNFLITISSHVGLAFTS
YYASRAALGLPFELRPSFTFLLAFMTVMGMTIGFAKDISDIEGDAKYGVSTVATKLG
ARNMTFVVSGVLLLNYVAAILAGIIWPQAFNSNVMLLSHAILAFCLIFQTRELALANY
ASAPSRQFFEFIWLLYYAEYFVYVFI
MPT21.17 (SEQ ID NO: 68)
MSDNSIATKILNFGHTCWKLQRPYVVKGMISIACGLFGKELLHNTNLI
SWGLMWKAFFALVPILSFNFFAAIMNQIYDVDIDRINKPDLPLVSGEMSIETAWILSII
VALTGLIVTIKLKSAPLFVFIYIFGIFAGFAYSVPPIRWKQNPSTNFLITISSHVGLAFTS
YYASRAALGLPFELRPSFTFLLAFMTVMGMTIAFAKDISDIEGDAKYGVSTVATKLG
ARNMTFVVSGVLLLNYVAAILAGIIWPQAFNSNVMLLSHAILAFCLIFQQRELALANY
ASAPSRQFFEFIWLLYYAEYFVYVFI
MPT21.18 (SEQ ID NO: 69)
MSDNSIATKILNFGHTCWKLQRPYVVKGMISIACGLFGKELLHNTNLI
SWGLMWKAFFALVPILSFNFFAAIMNQIYDVDIDRINKPDLPLVSGEMSIETAWILSII
VALTGLIVTIKLKSAPLFVFIYIFGIFAGFAYSVPPIRWKQNPSTNFLITISSHVGLAFTS
YYASRAALGLPFELRPSFTFLLAFMTVMGMTIAFAKDISDIEGDAKYGVSTVATKLG
ARNMTFVVSGVLLLNYVAAILAGIIWPQAFNSNVMLLSHAILAFCLIFQARELALANY
ASAPSRQFFEFIWLLYYAEYFVYVFI
MPT21.19 (SEQ ID NO: 70)
MSDNSIATKILNFGHTCWKLQRPYVVKGMISIACGLFGKELLHNTNLI
SWGLMWKAFFALVPILSFNFFAAIMNQIYDVDIDRINKPDLPLVSGEMSIETAWILSII
VALTGLIVTIKLKSAPLFVFIYIFGIFAGFAYSVPPIRWKQNPSTNFLITISSHVGLAFTS
YYASRAALGLPFELRPSFTFLLAFMTVMGMTIAFAKDISDIEGDAKYGVSTVATKLG
ARNMTFVVSGVLLLNYVAAILAGIIWPQAFNSNVMLLSHAILAFCLIFQTRELALANY
AAAPSRQFFEFIWLLYYAEYFVYVFI
MPT21.20 (SEQ ID NO: 71)
MSDNSIATKILNFGHTCWKLQRPYVVKGMISIACGLFGKELLHNTNLI
SWGLMWKAFFALVPILSFNFFAAIMNQIYDVDIDRINKPDLPLVSGEMSIETAWILSII
VALTGLIVTIKLKSAPLFVFIYIFGIFAGFAYSVPPIRWKQNPSTNFLITISSHVGLAFTS
YYASRAALGLPFELRPSFTFLLAFMTVMGMTIAFAKDISDIEGDAKYGVSTVATKLG
ARNMTFVVSGVLLLNYVAAILAGIIWPQAFNSNVMLLSHAILAFCLIFQTRELALANY
AGAPSRQFFEFIWLLYYAEYFVYVFI
MPT21.22 (SEQ ID NO: 72)
MSDNSIATKILNFGHTCWKLQRPYVVKGMISIACGLFGKELLHNTNLI
SWGLMWKAFFALVPILSFNFFAAIMNQIYDVDIDRINKPDLPLVSGEMSIETAWILSII
VALTGLIVTIKLKSAPLFVFIYIFGIFAGFAYSVPPIRWKQNPSTNFLITISSHVGLAFTS
YYASRAALGLPFELRPSFTFLLAFMTVMGMTIAFAKDISDIEGDAKYGVSTVATKLG
ARNMTFVVSGVLLLNYVAAILAGIIWPQAFNSNVMLLSHAILAFCLIFQTRELALANY
ASAPSRQFFEFLWLLYYAEYFVYVFI
MPT21 (SEQ ID NO: 73)
MSDNSIATKILNFGHTCWKLQRPYVVKGMISIACGLFGKELLHNTNLI
SWGLMWKAFFALVPILSFNFFAAIMNQIYDVDIDRINKPDLPLVSGEMSIETAWILSII
VALTGLIVTIKLKSAPLFVFIYIFGIFAGFAYSVPPIRWKQNPSTNFLITISSHVGLAFTS
YYASRAALGLPFELRPSFTFLLAFMTVMGMTIAFAKDISDIEGDAKYGVSTVATKLG
ARNMTFVVSGVLLLNYVAAILAGIIWPQAFNSNVMLLSHAILAFCLIFQTRELALANY
ASAPSRQFFEFIWLLYYAEYFVYVFI
MPT26 (SEQ ID NO: 74)
MSDNSIATKILNFGHTCWKLQRPYVVKGMISIACGLFGKELLHNTNLI
SWGLMWKAFFALVPILSFNFFAAIMNQIYDVDIDRINKPDLPLVSGEMSIETAWILSII
VALTGLIVTIKLKSAPLFVFIYIFGIFAGFAYSVPPIRWKQNPSTNFLITISSHVGLAFTS
YSATTSALGLPFVWRPAFSFIIAFMTVMGMTIAFAKDISDIEGDAKYGVSTVATKLGA
RNMTFVVSGVLLLNYVAAILAGIIWPQAFNSNVMLLSHAILAFWLILQTRDFALTNY
DPEAGRRFFEFIWLLYYAEYFVYVFI
MPT31 (SEQ ID NO: 75)
MSDNSIATKILNFGHACWKLQRPYVVKGMISIACGLFGRELLHNTNLI
SWGLMWKAFFALVPILSFNFFAAIMNQIYDLHIDRINKPDLPLASGEISVNTAWIMSII
VALTGLIVTIKLKSAPLFVFIYIFGIFAGFAYSVPPIRWKQNPSTNFLITISSHVGLAFTS
YYASRAALGLPFELRPSFTFLLAFMTVMGMTIAFAKDISDIEGDAKYGVSTVATKLG
ARNMTFVVSGVLLLNYLVSISIGIIWPQVFKSNIMILSHAILAFWLILQTRDFALTNYDP
EAGRRFFEFIWLLYYAEYLVYVFI
APT73.74 (SEQ ID NO: 76)
MDEVYAAVEQTSRLLDVPCSPDRFEPVWKAFGDQLPDSHLVFSMAA
GEAHRGELDFDFSLRPEGADPYTTALEHGFIEPTDHPVGSVLAEVGKRFAIASYGVEY
GVVGGFKKSYAFFPLDDFPPLAQFAEVPSVPPCLAGHVETLTRLGFDDKVSIIGVNYR
KNTLNVYLAASAVDTGDKLALLRAFGYPEPDARVRQFIERSFRLYPTFNWDSSAAER
ICFAVHTQQPGELPAPHDEPTEAFARQVPHVYEGGREFVSGVALAPSGASYYKLAAL
YQKARRCLH
APT73.77 (SEQ ID NO: 77)
MDEVYAAVEQTSRLLDVPCSPDRFEPVWKAFGDQLPDSHLVFSMAA
GEAHRGELDFDFSLRPEGADPYTTALEHGFIEPTDHPVGSVLAEVGKRFAIASYGVEY
GVVGGFKKSYAFFPLDDFPPLAQFAEVPSVPPCLAGHVETLTRLGFDDKVSIIGVNYR
KNTLNVYLAASAVDTGDKLALLRAFGYPEPDARVRQFIERSFRLYPTFNWDSSAAER
ICFAVHTQQPGELPAPHDEPTEAFARQVPHVYEGGREFVSGVALAPSGASYYKLAAL
YQKARRCLD
APT89.38 (SEQ ID NO: 78)
MDEVYAAVERTSRLLDVPCSPDRFEPVWKAFGDQLPDSHLVFSMAA
GEAHRGELDFDFSLRPEGADPYTTALEHGFIEPTDHPVGSVLAEVNKRCEIASYGVEY
GVVGGFKKSYAFFPLDDFPPLAEFARIPSVPPCLAGHVDTLTRLGLDDKVSAIGVNYR
KNTLNVYLAASAVATDDKLALLRAFGYPEPDARVRQFIERSFRLYPTFNWDSSAAER
ICFAVHTQQPGELPAPHDEPTEAFAREVPHVYEGGREFVSGVALAPSGAAYYKLAAE
YQKERRCL
F1 (SEQ ID NO: 79)
GGGGSGGGGSAEAAAKAEAAAKAGGGGSGGGGS
F2 (SEQ ID NO: 80)
GGAEAAAKEAAAKAGGSGGGSGGGGSGGS
F3 (SEQ ID NO: 81)
GGAEAAAKEAAAKAAEAAAKEAAAKAGGGSPGPGPGGGS
F4 (SEQ ID NO: 82)
GSSSSSSGSSSSSSGSSSSSSGSSSSSSGSSSSSSG
F5 (SEQ ID NO: 83)
GGGGSGGGGSGGGGS
F6 (SEQ ID NO: 84)
GGEAAAKEAAAKEAAAKGG
F7 (SEQ ID NO: 85)
GGAEAAAKEAAAKAPAPAPAG
F8 (SEQ ID NO: 86)
GTPTPTPTPTG
F9 (SEQ ID NO: 87)
GGGGSGGGGSGGGGSGGGGSGGGGSGGGGS
F10 (SEQ ID NO: 88)
GGAEAAAKEAAAKAAEAAAKEAAAKAAEAAAKEAAAKAAEAAAKEAAAKAGG
F11 (SEQ ID NO: 89)
GGAEAAAKEAAAKAGGSGGGSGGGGSGGSGGGGSGGGGS
F12 (SEQ ID NO: 90)
GGGGSGGGGS
F13 (SEQ ID NO: 91)
GGSGSAGSAAGSGEFGG
F14 (SEQ ID NO: 92)
GGAEAAAKEAAAKAPAPAPAEAAAKEAAAKAGG
F15 (SEQ ID NO: 93)
GGSGGAEAAAKEAAAKAGGSGG
F16 (SEQ ID NO: 94)
GGGSGGGSGGGSGGGGS
F17 (SEQ ID NO: 95)
GGGGS
F18 (SEQ ID NO: 96)
GGGGSLEDPAVWEAGKVVAKGVGTADITATTSNGLIASSEEADNAATS

1.-53. (canceled)

54. A cell producing an increased ratio of GPP to FPP as compared to a control cell, wherein the cell expresses a mutant farnesyl pyrophosphate synthase protein (FPPS), wherein the mutant FPPS is a mutant ERG20 or a mutant ERG20 homolog with at least one of a deletion, substitution or insertion at a position selected from positions corresponding to positions 88-90 of wild-type ERG20 (SEQ ID NO: 1), wherein the mutant ERG20 or the mutant ERG20 homolog does not contain a phenylalanine to tryptophan substitution at a position corresponding to position 88 of wild-type ERG20 (SEQ ID NO: 1).

55. The cell of claim 54, wherein the mutant FPPS is ERG20.A28 (SEQ ID NO: 22) or a mutant ERG20 or a mutant ERG20 homolog with an amino acid sequence having at least 90% identity to the amino acid sequence of wild type ERG20 (SEQ ID NO: 1).

56. The cell of claim 54, wherein the cell has altered expression of the mutant FPPS as compared to expression of wild-type FPPS in a control cell and/or the cell has reduced or no expression of wild-type ERG20.

57. The cell of claim 55, wherein the cell expresses ERG20.A28 and at least one of ERG20WW (i.e., ERG20.F88W.N119W), ERG20WW-MPT4.1, ERG20WW-MPT21.9, ERG20WW-APT73.81, or a farnesyl pyrophosphate synthase protein (FPPS) having a greater preference for GPP formation over FPP formation as compared to a FPPS control.

58. The cell of claim 54, wherein the cell has increased flux through the MVA pathway as compared to a control cell, wherein the cell over-expresses one or more native MVA pathway genes and/or expresses one or more transgenic MVA pathway genes selected from the group consisting of a feedback insensitive HMG-CoA synthase Erg13, a mevalonate kinase Erg12, and a NADH-dependent HMG-COA reductase.

59. The cell of claim 54, wherein the cell overexpresses mevalonate-5-phosphate decarboxylase (MPD), isopentenyl phosphokinase (IPK), and/or NADPH-dependent hydroxymethylglutaryl-CoA reductase.

60. The cell of claim 54, wherein the cell expresses one or more transgenic genes selected from limonene monoterpene synthase, myrcene monoterpene synthase, and cineole monoterpene synthase, wherein the cell has increased production of one or more monoterpenes as compared to a control cell.

61. The cell of claim 54, wherein the cell has an elevated level of DMAPP or GPP as compared to a control cell and/or the cell produces an elevated amount of one or more compounds prenylated with DMAPP as a donor, as compared to a control.

62. The cell of claim 54, wherein the cell overexpresses acetyl-CoA synthase (ACS) or overexpresses both ACS and acetyl-CoA carboxylase (ACC) as compared to a control cell, optionally, wherein the ACS is a mutant ACS with greater specificity for converting hexanoic acid to hexanoyl-CoA than the corresponding wild-type ACS and/or wherein the ACC is a mutant ACC with greater activity compared to wild-type ACC.

63. The cell of claim 54, wherein the cell produces CBGA, THCA, CBGVA, THCVA, and/or FCBGA and has increased CBGA, THCA, CBGVA, and/or THCVA production and/or reduced FCBGA production, as compared to a control cell.

64. The cell of claim 62, wherein the ACS is selected from the group consisting of ACS1 (SEQ ID NO: 41), ACS1.1 (SEQ ID NO: 7), or an ACS with an amino acid sequence having at least 90% identity to the amino acid sequence of ACS1.1, and the ACC is selected from the group consisting of ACC1 (SEQ ID NO: 44), ACC1.1 (SEQ ID NO: 45), or an ACC with an amino acid sequence having at least 90% identity to the amino acid sequence of ACC1.1.

65. The cell of claim 54, wherein the cell is a yeast cell or a bacterial cell, optionally wherein the yeast cell is a Yarrowia strain, a Saccharomyces strain, or a Pichia strain.

66. A method of producing CBGA, CBGVA, THCA, THCVA, or another cannabinoid derived from CBGA or CBGVA, a monoterpene, or a monoterpenoid comprising culturing a cell of claim 54 with a suitable carbon source under suitable conditions to produce the CBGA, CBGVA, THCA, THCVA, or another cannabinoid derived from CBGA or CBGVA, monoterpene, or monoterpenoid, and optionally isolating the CBGA, the CBGVA, the THCA, the THCVA, the another cannabinoid derived from CBGA or CBGVA, the monoterpene, or the monoterpenoid from the culture.

67. A mutant ERG20 or a mutant ERG20 homolog with an amino acid sequence at least about 90% homologous to the amino acid sequence of wild-type ERG20 (SEQ ID NO: 1) and comprising at least one insertion, deletion, or substitution at an amino acid position selected from amino acid positions 88-90 of wild-type ERG20 (SEQ ID NO: 1), wherein the mutant ERG20, the mutant ERG20 homolog or the mutant ERG20 ortholog does not contain a phenylalanine to tryptophan substitution at a position corresponding to position 88 of wild-type ERG20 (SEQ ID NO: 1).

68. The mutant ERG20 or the mutant ERG20 homolog of claim 67, wherein the mutant ERG20 has a polypeptide sequence selected from the group consisting of SEQ ID NOS: 8-31 or an amino acid sequence with at least 95% identity to an amino acid sequence selected from the group consisting of SEQ ID NOS: 8-31.

69. A cell overexpressing acetyl-CoA synthase (ACS) or overexpressing both ACS and acetyl-CoA carboxylase (ACC) as compared to a control cell, wherein the ACS is a mutant ACS with greater specificity for converting hexanoic acid to hexanoyl-CoA than the corresponding wild-type ACS and/or the ACC is a mutant ACC with greater activity compared to wild-type ACC, optionally wherein the cell is a yeast cell or a bacterial cell.

70. The cell of claim 69, wherein the ACC is selected from the group consisting of ACC1 (SEQ ID NO: 44), ACC1.1 (SEQ ID NO: 45), or an ACC with an amino acid sequence having at least 90% identity to the amino acid sequence of ACC1.1, and/or the ACS is selected from the group consisting of ACS1 (SEQ ID NO: 41), ACS1.1 (SEQ ID NO: 7), or an ACS with an amino acid sequence having at least 90% identity to the amino acid sequence of ACS1.1.

71. A mutant acetyl-CoA synthase (ACS) selected from ACS1.1 (SEQ ID NO: 7) or an ACS with an amino acid sequence 90% homologous to the amino acid sequence ACS1.1, wherein the mutant ACS has greater specificity for converting hexanoic acid to hexanoyl-CoA than the corresponding wild-type ACS.

72. A mutant acetyl-CoA carboxylase (ACC) selected from ACC1.1 (SEQ ID NO: 45) or an ACC with an amino acid sequence 90% homologous to the amino acid sequence of ACC1.1, wherein the mutant ACC has greater activity than the corresponding wild-type ACC.

73. A cell overexpressing pyruvate decarboxylase (PDC), aldehyde dehydrogenase (ALD), and/or one or more non-oxidative glycolysis pathway genes as compared to a control cell, optionally, wherein the cell is a yeast cell or a bacterial cell, and optionally wherein the cell has increased cannabinoid production compared to a control cell.


SEQUENCE LISTING (98 sequences)

SEQ ID NO: 1 - protein, 344 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFFLVSDDIMDESKTRRGQPCWYLKPKVGMIAINDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 2 - RNA, 20 nt, synthetic construct
  source 1..20: mol_type = other RNA
gcaggcgtttttcctcgtgt

SEQ ID NO: 3 - protein, 461 aa, synthetic construct
MAKNVGILAMDIYFPPTCVQQEALEAHDGASKGKYTIGLGQDCLAFCTELEDVISMSFNAVTSLLEKYKIDPKQIGRLEVGSETVIDKSKSIKTFLMQLFEKCGNTDVEGVDSTNACYGGTAALLNCVNWVESNSWDGRYGLVICTDSAVYAEGPARPTGGAAAIAMLIGPDAPIVFESKLRGSHMANVYDFYKPNLASEYPVVDGKLSQTCYLMALDSCYKHLCNKFEKLEGKEFSINDADYFVFHSPYNKLVQKSFARLLYNDFLRNASSIDEAAKEKFTPYSSLSLDESYQSRDLEKVSQQLAKTYYDAKVQPTTLVPKQVGNMYTASLYAAFASLVHNKHSDLAGKRVVMFSYGAGSTATMFSLRLCENQSPFSLSNIASVMDVGGKLKARHEYAPEKFVETMKLMEHRYGAKEFVTSKEGILDLLAPGTYYLKEVDSLYRRFYGKKGDDGSITNGH

SEQ ID NO: 4 - protein, 383 aa, synthetic construct
MTIGIDKISFFVPPYYIDMTALAEARNVDPGKFHIGIGQDQMAVNPISQDIVTFAANAAEAILTKEDKEAIDMVIVGTESSIDESKAAAVVLHRLMGIQPFARSFEIKEGCYGATAGLQLAKNHVALHPDKKVLVVAADIAKYGLNSGGEPTQGAGAVAMLVASEPRILALKEDNVMLTQDIYDFWRPTGHPYPMVDGPLSNETYIQSFAQVWDEHKKRTGLDFADYDALAFHIPYTKMGKKALLAKISDQTEAEQERILARYEESIIYSRRVGNLYTGSLYLGLISLLENATTLTAGNQIGLFSYGSGAVAEFFTGELVAGYQNHLQKETHLALLDNRTELSIAEYEAMFAETLDTDIDQTLEDELKYSISAINNTVRSYRN

SEQ ID NO: 5 - protein, 301 aa, synthetic construct
MVSCSAPGKIYLFGEHAVVYGETAIACAVELRTRVRAELNDSITIQSQIGRTGLDFEKHPYVSAVIEKMRKSIPINGVFLTVDSDIPVGSGLGSSAAVTIASIGALNELFGFGLSLQEIAKLGHEIEIKVQGAASPTDTYVSTFGGVVTIPERRKLKTPDCGIVIGDTGVFSSTKELVANVRQLRESYPDLIEPLMTSIGKISRIGEQLVLSGDYASIGRLMNVNQGLLDALGVNILELSQLIYSARAAGAFGAKITGAGGGGCMVALTAPEKCNQVAEAVAGAGGKVTITKPTEQGLKVD

SEQ ID NO: 6 - protein, 317 aa, synthetic construct
MTMASAPGKIILFGEHAVVSGTAALGGAIDLRARAIVQSLPGRILIETDDLSLRGFSLDLSTGEIRSASAAYATRYVSAVLKELGARDVRVMIESDIPPAAGLGSSASIVVATVAALNGHLGLELSQKEIAALSYRIEKEVQKGRGSPMDTALATYGGYQRIADDNQRLDLPPLEMVVGYTRLPHDTFSLVEKVQLLKERYPDLVGPIFQAIGAISERAAPLIREQRLKDLGELMDINHGLLEALGVGSRELSELVYAARNTGGALGAKLTGAGGGGCMIALPGMAGKDALLVALRQARGMAFAAMMGCEGVRLEVA

SEQ ID NO: 7 - protein, 657 aa, synthetic construct
MSEDHPAIHPPSEFKDNHPHFGGPHLDCLQDYHQLHKESIEDPKAFWKKMANELISWSTPFETVRSGGFEHGDVAWFPEGQLNASYNCVDRHAFANPDKPAIIFEADEPGQGRIVTYGELLRQVSQVAATLRSFGVQKGDTVAVYLPMIPEAIVTLLAITRIGAVHSVIFAGFSSGSLRDRINDAKSKVVVTTDASMRGGKTIDTKKIVDEALRDCPSVTHTLVFRRAGVENLAWTEGRDFWWHEEVVKHRPYLAPVPVASEDPIFLLYTSGSTGTPKGLAHATGGYLLGAALTAKYVFDIHGDDKLFTAGDVGWIGGHTYVLYGPLMLGATTVVFEGTPAYPSFSRYWDIVDDHKITHFYVAPTALRLLKRAGTHHIKHDLSSLRTLGSAGEPIAPDVWQWYNDNIGRGKAHICDTYGQTETGSHIIAPMAGVTPTKPGSASLPVFGIDPVIIDPVSGEELKGNNVEGVLALRSPWPSMARTVWNTHERYMETYLRPYPGYYFTGDGAARDNDGFYWIRGRVDDVVNVSGHRLSTAEIEAALIEHAQVSESAVVGVHDDLTGQAVNAFVALKNPVEDVDALRKELVVQVRKTIGPFAAPKNVIIVDDLPKTRSGKIMRRILRKVLAGEEDQLGDISTLANPDVVQTIIEVVHSLKK

SEQ ID NO: 8 - protein, 343 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFLVSDDIMDESKTRRGQPCWYLKPKVGMIAINDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 9 - protein, 343 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFFVSDDIMDESKTRRGQPCWYLKPKVGMIAINDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 10 - protein, 343 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFFLSDDIMDESKTRRGQPCWYLKPKVGMIAINDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 11 - protein, 341 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFSDDIMDESKTRRGQPCWYLKPKVGMIAINDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 12 - protein, 342 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFASDDIMDESKTRRGQPCWYLKPKVGMIAINDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 13 - protein, 342 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFRSDDIMDESKTRRGQPCWYLKPKVGMIAINDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 14 - protein, 342 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFNSDDIMDESKTRRGQPCWYLKPKVGMIAINDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 15 - protein, 342 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFDSDDIMDESKTRRGQPCWYLKPKVGMIAINDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 16 - protein, 342 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFCSDDIMDESKTRRGQPCWYLKPKVGMIAINDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 17 - protein, 342 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFQSDDIMDESKTRRGQPCWYLKPKVGMIAINDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 18 - protein, 342 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFESDDIMDESKTRRGQPCWYLKPKVGMIAINDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 19 - protein, 342 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFGSDDIMDESKTRRGQPCWYLKPKVGMIAINDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 20 - protein, 342 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFHSDDIMDESKTRRGQPCWYLKPKVGMIAINDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 21 - protein, 342 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFISDDIMDESKTRRGQPCWYLKPKVGMIAINDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 22 - protein, 342 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFLSDDIMDESKTRRGQPCWYLKPKVGMIAINDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 23 - protein, 342 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFKSDDIMDESKTRRGQPCWYLKPKVGMIAINDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 24 - protein, 342 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFMSDDIMDESKTRRGQPCWYLKPKVGMIAINDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 25 - protein, 342 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFFSDDIMDESKTRRGQPCWYLKPKVGMIAINDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 26 - protein, 342 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFPSDDIMDESKTRRGQPCWYLKPKVGMIAINDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 27 - protein, 342 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFSSDDIMDESKTRRGQPCWYLKPKVGMIAINDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 28 - protein, 342 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFTSDDIMDESKTRRGQPCWYLKPKVGMIAINDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 29 - protein, 342 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWSDDIMDESKTRRGQPCWYLKPKVGMIAINDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 30 - protein, 342 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFYSDDIMDESKTRRGQPCWYLKPKVGMIAINDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 31 - protein, 342 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFVSDDIMDESKTRRGQPCWYLKPKVGMIAINDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 32 - protein, 344 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQK

SEQ ID NO: 33 - protein, 692 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGAEAAAKEAAAKAGGSGGGSGGGGSGGSGGGGSGGGGSMSDNSIATKILNFGHTCWKLQRPYAVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 34 - protein, 692 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGAEAAAKEAAAKAGGSGGGSGGGGSGGSGGGGSGGGGSMSDNSIATKILNFGHTCWKLQRPYVVKGMISIACGLFGKELLHNTNLISWGLMWKAFFALVPILSFNFFASIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 35 - protein, 646 aa, synthetic construct
MSKAKFESVFPRISEELVQLLRDEGLPQDAVQWFSDSLQYNCVGGKLNRGLSVVDTYQLLTGKKELDDEEYYRLALLGWLIELLQAFWLVSDDIMDESKTRRGQPCWYLKPKVGMIAIWDAFMLESGIYILLKKHFRQEKYYIDLVELFHDISFKTELGQLVDLLTAPEDEVDLNRFSLDKHSFIVRYKTAYYSFYLPVVLAMYVAGITNPKDLQQAMDVLIPLGEYFQVQDDYLDNFGDPEFIGKIGTDIQDNKCSWLVNKALQKATPEQRQILEDNYGVKDKSKELVIKKLYDDMKIEQDYLDYEEEVVGDIKKKIEQVDESRGFKKEVLNAFLAKIYKRQKGGSGSAGSAAGSGEFGGMDEVYAAVEQTSRLLDVPCSPDRFEPVWKAFGDQLPDSHLVFSMAAGEAHRGELDFDFSLRPEGADPYTTALEHGFIEPTDHPVGSVLAEVGKRFAIASYGVEYGVVGGFKKSYAFFPLDDFPPLAQFAEVPSVPPCLAGHVETLTRLGFDDKVSIIGVNYRKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAALYQKGRRCLD

SEQ ID NO: 36 - protein, 310 aa, synthetic construct
MQLLNPPQKGKKAVEFDFNKYMDSKAMTVNEALNKAIPLRYPQKIYESMRYSLLAGGKRVRPVLCIAACELVGGTEELAIPTACAIEMIHTMSLMHDDLPCIDNDDLRRGKPTNHKIFGEDTAVTAGNALHSYAFEHIAVSTSKTVGADRILRMVSELGRATGSEGVMGGQMVDIASEGDPSIDLQTLEWIHIHKTAMLLECSVVCGAIIGGASEIVIERARRYARCVGLLFQVVDDILDVTKSSDELGKTAGKDLISDKATYPKLMGLEKAKEFSDELLNRAKGELSCFDPVKAAPLLGLADYVAFRQN

SEQ ID NO: 37 - protein, 371 aa, synthetic construct
MKDVSLSSFDAHDLDLDKFPEVVRDRLTQFLDAQELTIADIGAPVTDAVAHLRSFVLNGGKRIRPLYAWAGFLAAQGHKNSSEKLESVLDAAASLEFIQACALIHDDIIDSSDTRRGAPTVHRAVEADHRANNFEGDPEHFGVSVSILAGDMALVWAEDMLQDSGLSAEALARTRDAWRGMRTEVIGGQLLDIYLESHANESVELADSVNRFKTAAYTIARPLHLGASIAGGSPQLIDALLHYGHDIGIAFQLRDDLLGVFGDPAITGKPAGDDIREGKRTVLLALALQRADKQSPEAATAIRAGVGKVTSPEDIAVITEHIRATGAEEEVEQRISQLTESGLAHLDDVDIPDEVRAQLRALAIRSTERRM

SEQ ID NO: 38 - protein, 597 aa, synthetic construct
MHMAIPIKPAHYLHNSGRSYASQLCGFSSTSTRAAIARLPLCLRFRCSLQASDQRRSGNYSPSFWNADYILSLNSHYKDKSHMKRAGELIVQVKMVMGKETDPVVQLELIDDLQKLALSHHVEKEIKEILFKISTYDHKIMVERDLYSTALAFRLLRQYGFKVPQEVFDCFKNDNGEFKRSLSSDTKGLLQLYEASFLLTEGEMTLELAREFATKSLQEKLNEKTIDDDDDADTNLISCVRHSLDIPIHWRIQRPNASWWIDAYKRRSHMNPLVLELAKLDLNIFQAQFQQELKQDLGWWKNTCLAEKLPFVRDRLVECYFWCTGIIQPLQHENARVTLAKVNALITTLDDIYDVYGTLEELELFTEAIRRWDVSSIDHLPNYMQLCFLALNNFVDDTAYDVMKEKDINIIPYLRKSWLDLAETYLVEAKWFYSGHKPNLEEYLNNAWISISGPVMLCHVFFRVTDSITRETVESLFKYHDLIRYSSTILRLADDLGTSLEEVSRGDVPKSIQCYMNDNNASEEEARRHIRWLIAETWKKINEEVWSVDSPFCKDFIACAADMGRMAQFMYHNGDGHGIQNPQIHQQMTDILFEQWL

SEQ ID NO: 39 - protein, 555 aa, synthetic construct
MMVANKVSTSPDILRRSANYQPSIWNHDYIESLRIEYVGETCTRQINVLKEQVRMMLHKVVNPLEQLELIEILQRLGLSYHFEEEIKRILDGVYNNDHGGDTWKAENLYATALKFRLLRQHGYSVSQEVFNSFKDERGSFKACLCEDTKGMLSLYEASFFLIEGENILEEARDFSTKHLEEYVKQNKEKNLATLVNHSLEFPLHWRMPRLEARWFINIYRHNQDVNPILLEFAELDFNIVQAAHQADLKQVSTWWKSTGLVENLSFARDRPVENFFWTVGLIFQPQFGYCRRMFTKVFALITTIDDVYDVYGTLDELELFTDVVERWDINAMDQLPDYMKICFLTLHNSVNEMALDTMKEQRFHIIKYLKKAWVDLCRYYLVEAKWYSNKYRPSLQEYIENAWISIGAPTILVHAYFFVTNPITKEALDCLEEYPNIIRWSSIIARLADDLGTSTDELKRGDVPKAIQCYMNETGASEEGAREYIKYLISATWKKMNKDRAASSPFSHIFIEIALNLARMAQCLYQHGDGHGLGNRETKDRILSLLIQPIPLNKD

SEQ ID NO: 40 - protein, 547 aa, synthetic construct
MSLQTGNEIQTERRTGGYQPTLWDFSTIQSFDSEYKEEKHLMRAAGMIDQVKMMLQEEVDSIRRLELIDDLRRLGISCHFEREIVEILNSKYYTNNEIDERDLYSTALRFRLLRQYDFSVSQEVFDCFKNAKGTDFKPSLVDDTRGLLQLYEASFLSAQGEETLRLARDFATKFLQKRVLVDKDINLLSSIERALELPTHWRVQMPNARSFIDAYKRRPDMNPTVLELAKLDFNMVQAQFQQELKEASRWWNSTGLVHELPFVRDRIVECYYWTTGVVERRQHGYERIMLTKINALVTTIDDVFDIYGTLEELQLFTTAIQRWDIESMKQLPPYMQICYLALFNFVNEMAYDTLRDKGFDSTPYLRKVWVGLIESYLIEAKWYYKGHKPSLEEYMKNSWISIGGIPILSHLFFRLTDSIEEEAAESMHKYHDIVRASCTILRLADDMGTSLDEVERGDVPKSVQCYMNEKNASEEEAREHVRSLIDQTWKMMNKEMMTSSFSKYFVEVSANLARMAQWIYQHESDGFGMQHSLVNKMLRDLLFHRYE

SEQ ID NO: 41 - protein, 657 aa, synthetic construct
MSEDHPAIHPPSEFKDNHPHFGGPHLDCLQDYHQLHKESIEDPKAFWKKMANELISWSTPFETVRSGGFEHGDVAWFPEGQLNASYNCVDRHAFANPDKPAIIFEADEPGQGRIVTYGELLRQVSQVAATLRSFGVQKGDTVAVYLPMIPEAIVTLLAITRIGAVHSVIFAGFSSGSLRDRINDAKSKVVVTTDASMRGGKTIDTKKIVDEALRDCPSVTHTLVFRRAGVENLAWTEGRDFWWHEEVVKHRPYLAPVPVASEDPIFLLYTSGSTGTPKGLAHATGGYLLGAALTAKYVFDIHGDDKLFTAGDVGWITGHTYVLYGPLMLGATTVVFEGTPAYPSFSRYWDIVDDHKITHFYVAPTALRLLKRAGTHHIKHDLSSLRTLGSVGEPIAPDVWQWYNDNIGRGKAHICDTYWQTETGSHIIAPMAGVTPTKPGSASLPVFGIDPVIIDPVSGEELKGNNVEGVLALRSPWPSMARTVWNTHERYMETYLRPYPGYYFTGDGAARDNDGFYWIRGRVDDVVNVSGHRLSTAEIEAALIEHAQVSESAVVGVHDDLTGQAVNAFVALKNPVEDVDALRKELVVQVRKTIGPFAAPKNVIIVDDLPKTRSGKIMRRILRKVLAGEEDQLGDISTLANPDVVQTIIEVVHSLKK

SEQ ID NO: 42 - protein, 328 aa, synthetic construct
MSIIQNIIEKAKSDKKKIVLPEGAEPRTLKAAEIVLKEGIADLVLLGNEDEIRNAAKDLDISKAEIIDPVKSEMFDRYANDFYELRKNKGITLEKARETIKDNIYFGCMMVKEGYADGLVSGAIHATADLLRPAFQIIKTAPGAKIVSSFFIMEVPNCEYGENGVFLFADCAVNPSPNAEELASIAVQSANTAKNLLGFEPKVAMLSFSTKGSASHELVDKVRKATEIAKELMPDVAIDGELQLDAALVKEVAELKAPGSKVAGCANVLIFPDLQAGNIGYKLVQRLAKANAIGPITQGMGAPVNDLSRGCSYRDIVDVIATTAVQAQ

SEQ ID NO: 43 - protein, 796 aa, synthetic construct
MQSIIGKHKDEGKITPEYLKKIDAYWRAANFISVGQLYLLDNPLLREPLKPEHLKRKVVGHWGTIPGQNFIYAHLNRVIKKYDLDMIYVSGPGHGGQVMVSNSYLDGTYSEVYPNVSRDLNGLKKLCKQFSFPGGISSHMAPETPGSINEGGELGYSLAHSFGAVFDNPDLITACVVGDGEAETGPLATSWQANKFLNPVTDGAVLPILHLNGYKISNPTVLSRIPKDELEKFFEGNGWKPYFVEGEDPETMHKLMAETLDIVTEEILNIQKNARENNDCSRPKWPMIVLRTPKGWTGPKFVDGVPNEGSFRAHQVPLAVDRYHTENLDQLEEWLKSYKPEELFDENYRLIPELEELTPKGNKRMAANLHANGGLLLRELRTPDFRDYAVDVPTPGSTVKQDMIELGKYVRDVVKLNEDTRNFRIFGPDETMSNRLWAVFEGTKRQWLSEIKEPNDEFLSNDGRIVDSMLSEHLCEGWLEGYLLTGRHGFFASYEAFLRIVDSMITQHGKWLKVTSQLPWRKDIASLNLIATSNVWQQDHNGYTHQDPGLLGHIVDKKPEIVRAYLPADANTLLAVFDKCLHTKHKINLLVTSKHPRQQWLTMDQAVKHVEQGISIWDWASNDKGQEPDVVIASCGDTPTLEALAAVTILHEHLPELKVRFVNVVDMMKLLPENEHPHGLSDKDYNALFTTDKPVIFAFHGFAHLINQLTYHRENRNLHVHGYMEEGTITTPFDMRVQNKLDRFNLVKDVVENLPQLGNRGAHLVQLMNDKLVEHNQYIREVGEDLPEITNWQWHV

SEQ ID NO: 44 - protein, 2266 aa, synthetic construct
MRLQLRTLTRRFFSMASGSSTPDVAPLVDPNIHKGLASHFFGLNSVHTAKPSKVKEFVASHGGHTVINKVLIANNGIAAVKEIRSVRKWAYETFGDERAISFTVMATPEDLAANADYIRMADQYVEVPGGTNNNNYANVELIVDVAERSGVDAVWAGWGHASENPLLPESLAASPRKIVFIGPPGAAMRSLGDKISSTIVAQHAKVPCIPWSGTGVDEVVVDKSTNLVSVSEEVYTKGCTTGPKQGLEKAKQIGFPVMIKASEGGGGKGIRKVEREEDFEAAYHQVEGEIPGSPIFIMQLAGNARHLEVQLLADQYGNNISLFGRDCSVQRRHQKIIEEAPVTVAGQQTFTAMEKAAVRLGKLVGYVSAGTVEYLYSHEDDKFYFLELNPRLQVEHPTTEMVTGVNLPAAQLQIAMGIPLDRIKDIRLFYGVNPHTTTPIDFDFSGEDADKTQRRPVPRGHTTACRITSEDPGEGFKPSGGTMHELNFRSSSNVWGYFSVGNQGGIHSFSDSQFGHIFAFGENRSASRKHMVVALKELSIRGDFRTTVEYLIKLLETPDFEDNTITTGWLDELISNKLTAERPDSFLAVVCGAATKAHRASEDSIATYMASLEKGQVPARDILKTLFPVDFIYEGQRYKFTATRSSEDSYTLFINGSRCDIGVRPLSDGGILCLVGGRSHNVYWKEEVGATRLSVDSKTCLLEVENDPTQLRSPSPGKLVKFLVENGDHVRANQPYAEIEVMKMYMTLTAQEDGIVQLMKQPGSTIEAGDILGILALDDPSKVKHAKPFEGQLPELGPPTLSGNKPHQRYEHCQNVLHNILLGFDNQVVMKSTLQEMVGLLRNPELPYLQWAHQVSSLHTRMSAKLDATLAGLIDKAKQRGGEFPAKQLLRALEKEASSGEVDALFQQTLAPLFDLAREYQDGLAIHELQVAAGLLQAYYDSEARFCGPNVRDEDVILKLREENRDSLRKVVMAQLSHSRVGAKNNLVLALLDEYKVADQAGTDSPASNVHVAKYLRPVLRKIVELESRASAKVSLKAREILIQCALPSLKERTDQLEHILRSSVVESRYGEVGLEHRTPRADILKEVVDSKYIVFDVLAQFFAHDDPWIVLAALELYIRRACKAYSILDINYHQDSDLPPVISWRFRLPTMSSALYNSVVSSGSKTPTSPSVSRADSVSDFSYTVERDSAPARTGAIVAVPHLDDLEDALTRVLENLPKRGAGLAISVGASNKSAAASARDAAAAAASSVDTGLSNICNVMIGRVDESDDDDTLIARISQVIEDFKEDFEACSLRRITFSFGNSRGTYPKYFTFRGPAYEEDPTIRHIEPALAFQLELARLSNFDIKPVHTDNRNIHVYEATGKNAASDKRFFTRGIVRPGRLRENIPTSEYLISEADRLMSDILDALEVIGTTNSDLNHIFINFSAVFALKPEEVEAAFGGFLERFGRRLWRLRVTGAEIRMMVSDPETGSAFPLRAMINNVSGYVVQSELYAEAKNDKGQWIFKSLGKPGSMHMRSINTPYPTKEWLQPKRYKAHLMGTTYCYDFPELFRQSIESDWKKYDGKAPDDLMTCNELILDEDSGELQEVNREPGANNVGMVAWKFEAKTPEYPRGRSFIVVANDITFQIGSFGPAEDQFFFKVTELARKLGIPRIYLSANSGARIGIADELVGKYKVAWNDETDPSKGFKYLYFTPESLATLKPDTVVTTEIEEEGPNGVEKRHVIDYIVGEKDGLGVECLRGSGLIAGATSRAYKDIFTLTLVTCRSVGIGAYLVRLGQRAIQIEGQPIILTGAPAINKLLGREVYSSNLQLGGTQIMYNNGVSHLTARDDLNGVHKIMQWLSYIPASRGLPVPVLPHKTDVWDRDVTFQPVRGEQYDVRWLISGRTLEDGAFESGLFDKDSFQETLSGWAKGVVVGRARLGGIPFGVIGVETATVDNTTPADPANPDSIEMSTSEAGQVWYPNSAFKTSQAINDFNHGEALPLMILANWRGFSGGQRDMYNEVLKYGSFIVDALVDYKQPIMVYIPPTGELRGGSWVVVDPTINSDMMEMYADVESRGGVLEPEGMVGIKYRRDKLLDTMARLDPEYSSLKKQLEESPDSEELKVKLSVREKSLMPIYQQISVQFADLHDRAGRMEAKGVIREALVWKDARRFFFWRIRRRLVEEYLITKINSILPSCTRLECLARIKSWKPATLDQGSDRGVAEWFDENSDAVSARLSELKKDASAQSFASQLRKDRQGTLQGMKQALASLSEAERAELLKGL

SEQ ID NO: 45 - protein, 2266 aa, synthetic construct
MRLQLRTLTRRFFSMASGSSTPDVAPLVDPNIHKGLASHFFGLNSVHTAKPSKVKEFVASHGGHTVINKVLIANNGIAAVKEIRSVRKWAYETFGDERAISFTVMATPEDLAANADYIRMADQYVEVPGGTNNNNYANVELIVDVAERSGVDAVWAGWGHASENPLLPESLAASPRKIVFIGPPGAAMRSLGDKISSTIVAQHAKVPCIPWSGTGVDEVVVDKSTNLVSVSEEVYTKGCTTGPKQGLEKAKQIGFPVMIKASEGGGGKGIRKVEREEDFEAAYHQVEGEIPGSPIFIMQLAGNARHLEVQLLADQYGNNISLFGRDCSVQRRHQKIIEEAPVTVAGQQTFTAMEKAAVRLGKLVGYVSAGTVEYLYSHEDDKFYFLELNPRLQVEHPTTEMVTGVNLPAAQLQIAMGIPLDRIKDIRLFYGVNPHTTTPIDFDFSGEDADKTQRRPVPRGHTTACRITSEDPGEGFKPSGGTMHELNFRSSSNVWGYFSVGNQGGIHSFSDSQFGHIFAFGENRSASRKHMVVALKELSIRGDFRTTVEYLIKLLETPDFEDNTITTGWLDELISNKLTAERPDSFLAVVCGAATKAHRASEDSIATYMASLEKGQVPARDILKTLFPVDFIYEGQRYKFTATRSSEDSYTLFINGSRCDIGVRPLRDGGILCLVGGRSHNVYWKEEVGATRLRVDSKTCLLEVENDPTQLRSPSPGKLVKFLVENGDHVRANQPYAEIEVMKMYMTLTAQEDGIVQLMKQPGSTIEAGDILGILALDDPSKVKHAKPFEGQLPELGPPTLSGNKPHQRYEHCQNVLHNILLGFDNQVVMKSTLQEMVGLLRNPELPYLQWAHQVSSLHTRMSAKLDATLAGLIDKAKQRGGEFPAKQLLRALEKEASSGEVDALFQQTLAPLFDLAREYQDGLAIHELQVAAGLLQAYYDSEARFCGPNVRDEDVILKLREENRDSLRKVVMAQLSHSRVGAKNNLVLALLDEYKVADQAGTDSPASNVHVAKYLRPVLRKIVELESRASAKVSLKAREILIQCALPSLKERTDQLEHILRSSVVESRYGEVGLEHRTPRADILKEVVDSKYIVFDVLAQFFAHDDPWIVLAALELYIRRACKAYSILDINYHQDSDLPPVISWRFRLPTMSSALYNSVVSRGSKTPTSPSVSRADSVSDFSYTVERDSAPARTGAIVAVPHLDDLEDALTRVLENLPKRGAGLAISVGASNKSAAASARDAAAAAASSVDTGLSNICNVMIGRVDESDDDDTLIARISQVIEDFKEDFEACSLRRITFSFGNSRGTYPKYFTFRGPAYEEDPTIRHIEPALAFQLELARLSNFDIKPVHTDNRNIHVYEATGKNAASDKRFFTRGIVRPGRLRENIPTSEYLISEADRLMSDILDALEVIGTTNSDLNHIFINFSAVFALKPEEVEAAFGGFLERFGRRLWRLRVTGAEIRMMVSDPETGSAFPLRAMINNVSGYVVQSELYAEAKNDKGQWIFKSLGKPGSMHMRSINTPYPTKEWLQPKRYKAHLMGTTYCYDFPELFRQSIESDWKKYDGKAPDDLMTCNELILDEDSGELQEVNREPGANNVGMVAWKFEAKTPEYPRGRSFIVVANDITFQIGSFGPAEDQFFFKVTELARKLGIPRIYLSANSGARIGIADELVGKYKVAWNDETDPSKGFKYLYFTPESLATLKPDTVVTTEIEEEGPNGVEKRHVIDYIVGEKDGLGVECLRGSGLIAGATSRAYKDIFTLTLVTCRSVGIGAYLVRLGQRAIQIEGQPIILTGAPAINKLLGREVYSSNLQLGGTQIMYNNGVSHLTARDDLNGVHKIMQWLSYIPASRGLPVPVLPHKTDVWDRDVTFQPVRGEQYDVRWLISGRTLEDGAFESGLFDKDSFQETLSGWAKGVVVGRARLGGIPFGVIGVETATVDNTTPADPANPDSIEMSTSEAGQVWYPNSAFKTSQAINDFNHGEALPLMILANWRGFSGGQRDMYNEVLKYGSFIVDALVDYKQPIMVYIPPTGELRGGSWVVVDPTINSDMMEMYADVESRGGVLEPEGMVGIKYRRDKLLDTMARLDPEYSSLKKQLEESPDSEELKVKLSVREKSLMPIYQQISVQFADLHDRAGRMEAKGVIREALVWKDARRFFFWRIRRRLVEEYLITKINSILPSCTRLECLARIKSWKPATLDQGSDRGVAEWFDENSDAVSARLSELKKDASAQSFASQLRKDRQGTLQGMKQALASLSEAERAELLKGL

SEQ ID NO: 46 - protein, 803 aa, synthetic construct
MKTVVIIDALRTPIGKYKGSLSQVSAVDLGTHVTTQLLKRHSTISEEIDQVIFGNVLQAGNGQNPARQIAINSGLSHEIPAMTVNEVCGSGMKAVILAKQLIQLGEAEVLIAGGIENMSQAPKLQRFNYETESYDAPFSSMMYDGLTDAFSGQAMGLTAENVAEKYHVTREEQDQFSVHSQLKAAQAQAEGIFADEIAPLEVSGTLVEKDEGIRPNSSVEKLGTLKTVFKEDGTVTAGNASTINDGASALIIASQEYAEAHGLPYLAIIRDSVEVGIDPAYMGISPIKAIQKLLARNQLTTEEIDLYEINEAFAATSIVVQRELALPEEKVNIYGGGISLGHAIGATGARLLTSLSYQLNQKEKKYGVASLCIGGGLGLAMLLERPQQKKNSRFYQMSPEERLASLLNEGQISADTKKEFENTALSSQIANHMIENQISETEVPMGVGLHLTVDETDYLVPMATEEPSVIAALSNGAKIAQGFKTVNQQRLMRGQIVFYDVADAESLIDELQVRETEIFQQAELSYPSIVKRGGGLRDLQYRAFDESFVSVDFLVDVKDAMGANIVNAMLEGVAELFREWFAEQKILFSILSNYATESVVTMKTAIPVSRLSKGSNGREIAEKIVLASRYASLDPYRAVTHNKGIMNGIEAVVLATGNDTRAVSASCHAFAVKEGRYQGLTSWTLDGEQLIGEISVPLALATVGGATKVLPKSQAAADLLAVTDAKELSRVVAAVGLAQNLAALRALVSEGIQKGHMALQARSLAMTVGATGKEVEAVAQQLKRQKTMNQDRALAILNDLRKQ

SEQ ID NO: 47 - protein, 309 aa, synthetic construct
MSDNSIATKILNFGHTCWKLQRPYAVKGMISIACGLFGRELFNNRHLFSWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQYPFTNFLITISSHVGLAFTSYSATTSALGLPFVWRPAFSFIIAFMTVMGMTIAFAKDISDIEGDAKYGVSTVATKLGARNMTFVVSGVLLLNYLVSISIGIIWPQVFKSNIMILSHAILAFCLIFQTRELALANYASAPSRQFFEFIWLLYYAEYFVYVFI

SEQ ID NO: 48 - protein, 309 aa, synthetic construct
MSDNSIATKILNFGHTCWKLQRPYVVKGMISIACGLFGKELLHNTNLISWGLMWKAFFALVPILSFNFFASIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 49 - protein, 285 aa, synthetic construct
MDEVYAAVEQTSRLLDVPCSPDRFEPVWKAFGDQLPDSHLVFSMAAGEAHRGELDFDFSLRPEGADPYTTALEHGFIEPTDHPVGSVLAEVGKRFAIASYGVEYGVVGGFKKSYAFFPLDDFPPLAQFAEVPSVPPCLAGHVETLTRLGFDDKVSIIGVNYRKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAALYQKGRRCLD

SEQ ID NO: 50 - protein, 999 aa, synthetic construct
MLQAAIGKIVGFAVNRPIHTVVLTSIVASTAYLAILDIAIPGFEGTQPISYYHPAAKSYDNPADWTHIAEADIPSDAYRLAFAQIRVSDVQGGEAPTIPGAVAVSDLDHRIVMDYKQWAPWTASNEQIASENHIWKHSFKDHVAFSWIKWFRWAYLRLSTLIQGADNFDIAVVALGYLAMHYTFFSLFRSMRKVGSHFWLASMALVSSTFAFLLAVVASSSLGYRPSMITMSEGLPFLVVAIGFDRKVNLASEVLTSKSSQLAPMVQVITKIASKALFEYSLEVAALFAGAYTGVPRLSQFCFLSAWILIFDYMFLLTFYSAVLAIKFEINHIKRNRMIQDALKEDGVSAAVAEKVADSSPDAKLDRKSDVSLFGASGAIAVFKIFMVLGFLGLNLINLTAIPHLGKAAAAAQSVTPITLSPELLHAIPASVPVVVTFVPSVVYEHSQLILQLEDALTTFLAACSKTIGDPVISKYIFLCLMVSTALNVYLFGATREVVRTQSVKVVEKHVPIVIEKPSEKEEDTSSEDSIELTVGKQPKPVTETRSLDDLEAIMKAGKTKLLEDHEVVKLSLEGKLPLYALEKQLGDNTRAVGIRRSIISQQSNTKTLETSKLPYLHYDYDRVFGACCENVIGYMPLPVGVAGPMNIDGKNYHIPMATTEGCLVASTMRGCKAINAGGGVTTVLTQDGMTRGPCVSFPSLKRAGAAKIWLDSEEGLKSMRKAFNSTSRFARLQSLHSTLAGNLLFIRFRTTTGDAMGMNMISKGVEHSLAVMVKEYGFPDMDIVSVSGNYCTDKKPAAINWIEGRGKSVVAEATIPAHIVKSVLKSEVDALVELNISKNLIGSAMAGSVGGFNAHAANLVTAIYLATGQDPAQNVESSNCITLMSNVDGNLLISVSMPSIEVGTIGGGTILEPQGAMLEMLGVRGPHIETPGANAQQLARIIASGVLAAELSLCSALAAGHLVQSHMTHNRSQAPTPAKQSQADLQRLQNGSNICIRS

SEQ ID NO: 51 - protein, 505 aa, synthetic construct
MREVVRTQSVKVVEKHVPIVIEKPSEKEEDTSSEDSIELTVGKQPKPVTETRSLDDLEAIMKAGKTKLLEDHEVVKLSLEGKLPLYALEKQLGDNTRAVGIRRSIISQQSNTKTLETSKLPYLHYDYDRVFGACCENVIGYMPLPVGVAGPMNIDGKNYHIPMATTEGCLVASTMRGCKAINAGGGVTTVLTQDGMTRGPCVSFPSLKRAGAAKIWLDSEEGLKSMRKAFNSTSRFARLQSLHSTLAGNLLFIRFRTTTGDAMGMNMISKGVEHSLAVMVKEYGFPDMDIVSVSGNYCTDKKPAAINWIEGRGKSVVAEATIPAHIVKSVLKSEVDALVELNISKNLIGSAMAGSVGGFNAHAANLVTAIYLATGQDPAQNVESSNCITLMSNVDGNLLISVSMPSIEVGTIGGGTILEPQGAMLEMLGVRGPHIETPGANAQQLARIIASGVLAAELSLCSALAAGHLVQSHMTHNRSQAPTPAKQSQADLQRLQNGSNICIRS

SEQ ID NO: 52 - protein, 270 aa, synthetic construct
MTTSYSDKIKSISASSVAQQFPEVAPIADVSKASRPSTESSDSSAKLFDGHDEEQIKLMDEICVVLDWDDKPIGGASKKCCHLMDNINDGLVHRAFSVFMFNDRGELLLQQRAAEKITFANMWTNTCCSHPLAVPSEMGGLDLESRIQGAKNAAVRKLEHELGIDPKAVPADKFHFLTRIHYAAPSSGPWGEHEIDYILFVRGDPELKVVANEVRDTVWVSQQGLKDMMADPKLVFTPWFRLICEQALFPWWDQLDNLPAGDDEIRRWIK

SEQ ID NO: 53 - protein, 309 aa, synthetic construct
MSDNSIATKILNFGHTCWKLQRPFVVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 54 - protein, 309 aa, synthetic construct
MSDNSIATKILNFGHTCWKLQRPMVVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 55 - protein, 309 aa, synthetic construct
MSDNSIATKILNFGHTCWKLQRPYAVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 56 - protein, 309 aa, synthetic construct
MSDNSIATKILNFGHTCWKLQRPYVVKGAISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 57 - protein, 309 aa, synthetic construct
MSDNSIATKILNFGHTCWKLQRPYVVKGMITIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 58 - protein, 309 aa, synthetic construct
MSDNSIATKILNFGHTCWKLQRPYVVKGMIVIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 59 - protein, 309 aa, synthetic construct
MSDNSIATKILNFGHTCWKLQRPYVVKGMIAIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 60 - protein, 309 aa, synthetic construct
MSDNSIATKILNFGHTCWKLQRPYVVKGMISIACGLFGKELLHNTNLISWGLMWKAFFALVPILSFNFFAGIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 61 - protein, 309 aa, synthetic construct
MSDNSIATKILNFGHTCWKLQRPYVVKGMISIACGLFGKELLHNTNLISWGLMWKAFFALVPILSFNFFAAIMNQIYDVDMDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 62 - protein, 309 aa, synthetic construct
MSDNSIATKILNFGHTCWKLQRPYVVKGMISIACGLFGKELLHNTNLISWGLMWKAFFALVPILSFNFFAAIMNQIYDVDIDRVNKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 63 - protein, 309 aa, synthetic construct
MSDNSIATKILNFGHTCWKLQRPYVVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFEITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 64 - protein, 309 aa, synthetic construct
MSDNSIATKILNFGHTCWKLQRPYVVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITIASHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 65 - protein, 309 aa, synthetic construct
MSDNSIATKILNFGHTCWKLQRPYVVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITIGSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 66 - protein, 309 aa, synthetic construct
MSDNSIATKILNFGHTCWKLQRPYVVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITIVSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 67 - protein, 309 aa, synthetic construct
MSDNSIATKILNFGHTCWKLQRPYVVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIGFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 68 - protein, 309 aa, synthetic construct
MSDNSIATKILNFGHTCWKLQRPYVVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQQRELALANYASAPSRQFFEFIWLLYYAEYFVYVFI

SEQ ID NO: 69 - protein, 309 aa, synthetic construct
MSDNSIATKILNFGHTCWKLQRPYVVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQARELALANYASAPSRQFFEFIWLLYYAEYFVYVFI

SEQ ID NO: 70 - protein, 309 aa, synthetic construct
MSDNSIATKILNFGHTCWKLQRPYVVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYAAAPSRQFFEFIWLLYYAEYFVYVFI

SEQ ID NO: 71 - protein, 309 aa, synthetic construct
MSDNSIATKILNFGHTCWKLQRPYVVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYAGAPSRQFFEFIWLLYYAEYFVYVFI

SEQ ID NO: 72 - protein, 309 aa, synthetic construct
MSDNSIATKILNFGHTCWKLQRPYVVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFLWLLYYAEYFVYVFI

SEQ ID NO: 73 - protein, 309 aa, synthetic construct
MSDNSIATKILNFGHTCWKLQRPYVVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYVAAILAGIIWPQAFNSNVMLLSHAILAFCLIFQTRELALANYASAPSRQFFEFIWLLYYAEYFVYVFI

SEQ ID NO: 74 - protein, 309 aa, synthetic construct
MSDNSIATKILNFGHTCWKLQRPYVVKGMISIACGLFGKELLHNTNLISWGLMWKAFFALVPILSFNFFAAIMNQIYDVDIDRINKPDLPLVSGEMSIETAWILSIIVALTGLIVTIKLKSAPLFVFIYIFGIFAGFAYSVPPIRWKQNPSTNFLITISSHVGLAFTSYSATTSALGLPFVWRPAFSFIIAFMTVMGMTIAFAKDISDIEGDAKYGVSTVATKLGARNMTFVVSGVLLLNYVAAILAGIIWPQAFNSNVMLLSHAILAFWLILQTRDFALTNYDPEAGRRFFEFIWLLYYAEYFVYVFI

SEQ ID NO: 75 - protein, 309 aa, synthetic construct
MSDNSIATKILNFGHACWKLQRPYVVKGMISIACGLFGRELLHNTNLISWGLMWKAFFALVPILSFNFFAAIMNQIYDLHIDRINKPDLPLASGEISVNTAWIMSIIVALTGLIVTIKLKSAPLFVFIYIFGIFAGFAYSVPPIRWKQNPSTNFLITISSHVGLAFTSYYASRAALGLPFELRPSFTFLLAFMTVMGMTIAFAKDISDIEGDAKYGVSTVATKLGARNMTFVVSGVLLLNYLVSISIGIIWPQVFKSNIMILSHAILAFWLILQTRDFALTNYDPEAGRRFFEFIWLLYYAEYLVYVFI

SEQ ID NO: 76 - protein, 285 aa, synthetic construct
MDEVYAAVEQTSRLLDVPCSPDRFEPVWKAFGDQLPDSHLVFSMAAGEAHRGELDFDFSLRPEGADPYTTALEHGFIEPTDHPVGSVLAEVGKRFAIASYGVEYGVVGGFKKSYAFFPLDDFPPLAQFAEVPSVPPCLAGHVETLTRLGFDDKVSIIGVNYRKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAALYQKARRCLH

SEQ ID NO: 77 - protein, 285 aa, synthetic construct
MDEVYAAVEQTSRLLDVPCSPDRFEPVWKAFGDQLPDSHLVFSMAAGEAHRGELDFDFSLRPEGADPYTTALEHGFIEPTDHPVGSVLAEVGKRFAIASYGVEYGVVGGFKKSYAFFPLDDFPPLAQFAEVPSVPPCLAGHVETLTRLGFDDKVSIIGVNYRKNTLNVYLAASAVDTGDKLALLRAFGYPEPDARVRQFIERSFRLYPTFNWDSSAAERICFAVHTQQPGELPAPHDEPTEAFARQVPHVYEGGREFVSGVALAPSGASYYKLAALYQKARRCLD

SEQ ID NO: 78 - protein, 284 aa, synthetic construct
MDEVYAAVERTSRLLDVPCSPDRFEPVWKAFGDQLPDSHLVFSMAAGEAHRGELDFDFSLRPEGADPYTTALEHGFIEPTDHPVGSVLAEVNKRCEIASYGVEYGVVGGFKKSYAFFPLDDFPPLAEFARIPSVPPCLAGHVDTLTRLGLDDKVSAIGVNYRKNTLNVYLAASAVATDDKLALLRAFGYPEPDARVRQFIERSFRLYPTFNWDSSAAERICFAVHTQQPGELPAPHDEPTEAFAREVPHVYEGGREFVSGVALAPSGAAYYKLAAEYQKERRCL

SEQ ID NO: 79 - protein, 33 aa, synthetic construct
GGGGSGGGGSAEAAAKAEAAAKAGGGGSGGGGS

SEQ ID NO: 80 - protein, 29 aa, synthetic construct
GGAEAAAKEAAAKAGGSGGGSGGGGSGGS

SEQ ID NO: 81 - protein, 39 aa, synthetic construct
GGAEAAAKEAAAKAAEAAAKEAAAKAGGGSPGPGPGGGS

SEQ ID NO: 82 - protein, 36 aa, synthetic construct
GSSSSSSGSSSSSSGSSSSSSGSSSSSSGSSSSSSG

SEQ ID NO: 83 - protein, 15 aa, synthetic construct
GGGGSGGGGSGGGGS

SEQ ID NO: 84 - protein, 19 aa, synthetic construct
GGEAAAKEAAAKEAAAKGG

SEQ ID NO: 85 - protein, 21 aa, synthetic construct
GGAEAAAKEAAAKAPAPAPAG

SEQ ID NO: 86 - protein, 11 aa, synthetic construct
GTPTPTPTPTG

SEQ ID NO: 87 - protein, 30 aa, synthetic construct
GGGGSGGGGSGGGGSGGGGSGGGGSGGGGS

SEQ ID NO: 88 - protein, 52 aa, synthetic construct
GGAEAAAKEAAAKAAEAAAKEAAAKAAEAAAKEAAAKAAEAAAKEAAAKAGG

SEQ ID NO: 89 - protein, 39 aa, synthetic construct
GGAEAAAKEAAAKAGGSGGGSGGGGSGGSGGGGSGGGGS

SEQ ID NO: 90 - protein, 10 aa, synthetic construct
GGGGSGGGGS

SEQ ID NO: 91 - protein, 17 aa, synthetic construct
GGSGSAGSAAGSGEFGG

SEQ ID NO: 92 - protein, 33 aa, synthetic construct
GGAEAAAKEAAAKAPAPAPAEAAAKEAAAKAGG

SEQ ID NO: 93 - protein, 22 aa, synthetic construct
GGSGGAEAAAKEAAAKAGGSGG

SEQ ID NO: 94 - protein, 17 aa, synthetic construct
GGGSGGGSGGGSGGGGS

SEQ ID NO: 95 - protein, 5 aa, synthetic construct
GGGGS

SEQ ID NO: 96 - protein, 46 aa, synthetic construct
GGGGSLEDPAVWEAGKVVAKGVGTADITATTSNGLIASSEEADNAA

SEQ ID NO: 97 - protein, 23 aa, synthetic construct
ELLQAFFLVSDDIMDESKTRRGQ

SEQ ID NO: 98 - protein, 21 aa, synthetic construct
ELLQAFLSDDIMDESKTRRGQ